(19) United States
(12) Patent Application Publication
(10) Pub. No.: US 2007/0203236 A1
(43) Pub. Date: Aug. 30, 2007

(54) NOVEL ANTAGONISTS OF THE HUMAN FATTY ACID SYNTHASE THIOESTERASE

(76) Inventors: Jeffrey W. Smith, Del Mar, CA (US); Robyn D. Richardson, San Diego, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/622,339

(22) Filed: Jan. 11, 2007

Related U.S. Application Data
(60) Provisional application No. 60/758,103, filed on Jan. 11, 2006.

(51) Int. Cl. A61K 31/202

(52) U.S. Cl. 514/560

(57) ABSTRACT

The present invention provides for compounds of formula (I)-(XIII), as well as pharmaceutically acceptable salts thereof, metabolites thereof, pro-drugs thereof, and pharmaceutical kits that include such compounds. The present invention also provides for the compounds of formula (I)-(XIII) for use in medical therapy or diagnosis. The present invention also provides for the use of the compounds of formula (I)-(XIII) in treating cancer in mammals (e.g., humans), as well inhibiting tumor cell growth in such mammals. The present invention also provides for methods of inhibiting FAS. The methods include contacting FAS with an effective amount of a compound of formula (I)-(XIII). The present invention also provides for methods of inhibiting the TE domain of the FAS. The methods include contacting the thioesterase TE domain of the FAS with an effective amount of a compound of formula (I)-(XIII). The present invention also provides for methods of treating cancer in mammals, as well as methods of inhibiting tumor cell growth in such mammals. The methods include administering a compound of formula (I)-(XIII) to a mammal in need of such treatment.

### CROSS-REFERENCE TO RELATED APPLICATIONS

This application claims the benefit of the filing date of Ser. No. 60/758,103, filed Jan. 11, 2006, the disclosure of which is incorporated by reference herein.

### STATEMENT OF GOVERNMENT RIGHTS

The invention was made, at least in part, with a grant from the Government of the United States of America (grant nos. RR020843 and CA108959 from the National Institutes of Health and grant nos. DAMD17-02-0693 and W81XWH-04-1-0515 from the Department of Defense). The Government may have certain rights to the invention.

### BACKGROUND

There is growing interest in fatty acid synthase (FAS) as an anti-tumor target because it is up-regulated and linked to poor prognosis in many solid tumors including those of the breast (Alo et al., 1996; Nakamura et al., 1999; Wang et al., 2004), prostate (Swinnen et al., 2002; Rossi et al., 2003; Bandyopadhyay et al., 2005), and ovaries (Pizer et al., 1996; Gansler et al., 1997; Tsuji et al., 2004). Moreover, inhibition of FAS with active site modifying agents blocks tumor cell proliferation, elicits tumor cell death and prevents tumor growth in animal models. It was recently reported, that orlistat, an approved obesity drug, antagonizes the thioesterase (TE) domain of FAS (Kridel et al., 2004), which is a serine hydrolase. By virtue of its ability to inhibit FAS, orlistat blocks tumor cell proliferation and the growth of tumor xenografts in mice (Kridel et al., 2004; Knowles et al., 2004). While orlistat is given to patients orally, systemic bioavailability is minimal. The drug is largely confined to the gut, where it inhibits pancreatic lipase, blocking the absorption of dietary fats, and preventing weight gain (Hadvary et al., 1991; Luthi-Peng et al., 1992).

FAS has six separate enzymatic pockets that act sequentially to condense acetyl CoA and malonyl CoA, ultimately generating a palmitoyl-acyl carrier protein (ACP) complex (Wakil, 1989) from which palmitate is liberated by the C-terminal TE. The close proximity of the palmitate-bound ACP to the TE results in a high effective concentration of substrate. Therefore, to inhibit this interaction, an unusually high concentration of a competitive, reversible inhibitor would be needed to achieve a therapeutic effect.

### SUMMARY OF THE INVENTION

The invention provides compounds and methods useful to inhibit a TE containing polypeptide. As described below, more than 35,000 compounds were screened for antagonists of the FAS TE domain or a pathogen-specific TE containing polypeptide using a fluorogenic high throughput assay. Non-competitive inhibitors that interact with the TE at a site distinct from the substrate-binding site were identified. The TE antagonists of the invention include pyrazolidines, pyrozoles, diphenyl acetamides, pyrrolidiones, thioxopyridmidine diones, quinolones and barbituric acid derivatives. In particular, 19 thio-barbituric or barbituric acid derivatives, 8 of which have an IC₅₀ of less than 5 μM in vitro, were identified. The most potent of these barbituric acid derivatives blocked the activity of the human FAS holoenzyme and were cytotoxic to breast cancer cells. The invention thus provides serine hydrolase inhibitors that bind reversibly to the enzyme, act as partial non-competitive inhibitors, and elicit tumor cell death.

Also provided are antagonists of TE containing polypeptides of pathogens, e.g., Bacillus anthracis, Yersinia pestis, Vibrio spp., Salmonella spp., Listeria spp. and Mycobacterium spp. For example, pyrazolidines, pyrozoles, diphenyl acetamides, pyrrolidiones, thioxopyridmidine diones, and quinolones were found to inhibit Y. pestis YbtT.

In one embodiment, the present invention provides for novel compounds of formula (I)-(XIII), as well as pharmaceutically acceptable salts thereof, metabolites thereof, pro-drugs thereof, and pharmaceutical kits that includes such compounds.

The present invention also provides for a compound of formula (I)-(XIII), for use in medical therapy or diagnosis.

The present invention further provides for the use of a compound of formula (I)-(XIII), for the manufacture of a medicament for treating cancer in mammals (e.g., humans), as well as inhibiting tumor cell growth in such mammals.

The present invention also provides for methods of inhibiting or treating cancer in mammals, as well as methods of inhibiting tumor cell growth in such mammals. The methods include administering a compound of formula (I)-(XIII) to a mammal in need of such treatment.

The tumor can be a solid tumor and can be located, e.g, in the ovary, breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary gland, or the heart. Additionally, the compounds of the present invention can be administered locally or systemically, alone or in combination with one or more anti-cancer agents.

Further provided are methods of inhibiting FAS. The methods include contacting FAS with an effective amount of a compound of formula (I)-(XIII).

The present invention also provides for methods of inhibiting a TE containing polypeptide. The methods include contacting the TE containing polypeptide, e.g., FAS or other serine hydrolase, with an effective amount of a compound of formula (I)-(XIII).

Further provided are compounds useful to inhibit or treat an infection of a mammal by a pathogen, e.g., a bacteria, fungi, virus or other non-eukaryotic pathogen. In addition, methods of inhibiting or treating an infection of a mammal by a pathogen with one or more of the compounds are provided. Also provided are methods of identifying compounds that selectively inhibit a TE containing polypeptide of a pathogen relative to one or more TE containing polypeptides of a mammal, e.g., a human. As used herein, a compound that “selectively inhibits” a TE containing polypeptide includes a compound that inhibits a particular TE containing polypeptide by at least about 2-fold more than a different TE containing polypeptide.

### BRIEF DESCRIPTION OF THE FIGURES

FIG. 1. Identification of TE antagonists from a primary screen of 36,500 compounds. Recombinant FAS TE was used to screen 36,500 drug-like compounds. The screening assay was based on the turnover of the 4-MUH substrate by the TE, which yielded fluorescence upon liberation of the 4-MU. All compounds were initially screened at a final concentration of approximately 12.5 μM. The primary hits (116) from this screen were retested revealing 18 compounds with apparent Kᵢ<1.0 μM.

FIG. 2. Barbituric acids are partial non-competitive TE inhibitors. Kinetic characterization of recombinant TE (500 mM) activity (A) following treatment with DMSO (▪) or compound (1) at 2 μM (▾), 4 μM (♦), and 10 μM (▴), and (B) DMSO (□) or compound (7) at 1 μM (×), 2 μM (∘), and, 4 μM (⋄). The X-intercept for each condition is −1/Kₘ. (C) Activity of recombinant TE (500 to 1250 nM) treated with DMSO (▪) compared to compound (1) at 10 μM (●), classified the non-competitive inhibition as reversible or irreversible. Intersection of plots at the x-axis indicates reversible inhibition. (D) Data from FAS inhibition by compound (1) was replotted versus Kₘ/Vₘₐₓ₍ᵢ₎ to distinguish between pure and partial non-competitive inhibition. Hyberbolic plots indicate partial non-competitive inhibition. All treatments were preformed in triplicate; error bars indicate SD.

FIG. 3. Effects of barbituric acid derivatives on cellular FAS. (A) A representative experiment showing inhibition of FP-BODIPY probe binding by increasing concentrations of (2) (top) and (3) (bottom). MB-MDA-435 cell lysates were pre-incubated with test compounds (0 to 100 μM) for 30 minutes, followed by addition of 50 nM probe for 30 minutes. Samples were resolved by electrophoresis and visualized by scanning at 505 nm. V=vehicle only. (B) FAS in vitro activity was measured as the incorporation of [¹⁴C] malonyl-CoA over 2 hours following preincubation of MB-MDA-435 cell lysates with (2) (▴) or (3) (▪) at 0 to 50 μM for 60 minutes. De novo fatty acids were extracted and quantified by scintillation. Treatments were preformed in duplicate, error bars indicate SD.

FIG. 4. Human TE containing polypeptides.

FIG. 5. Inhibition of human FAS TE or Yersinia YbtT by select compounds.

FIG. 6. Inhibition of human FAS TE or Yersinia YbtT by select compounds.

FIG. 7. Pathogen proteins with a TE domain.

### DETAILED DESCRIPTION OF THE INVENTION

Reference will now be made in detail to embodiments of the invention. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims.

Thioesterases

Thioesterases (TEs) use an Asp/His/Ser catalytic triad to hydrolyze substrates. There are more than 1000 TEs, spanning prokaryotes, fungi, and eukaryotes. Human FAS is the sole enzyme responsible for the conversion of dietary carbohydrate to palmitate, the precursor for most fatty acids. FAS contains six enzymatic pockets that condense acetyl CoA and malonyl CoA, to generate palmitate. The C-terminal domain of FAS contains a TE that liberates palmitate from the enzyme.

Orlistat, a drug approved for treating obesity, is an unexpectedly potent antagonist of the TE of FAS. Moreover, Orlistat elicits cytostatic and cytotoxic effects on tumor cells, inhibits proliferation of human umbilical vein endothelial cells and inhibits neovascularization. However, Orlistat contains a reactive pharmacophore (a β-lactone) that is not be optimal for drug development as the reactive group leads to dead end inhibition of FAS. Thus, removal of the drug is dependent upon the half-life of FAS; halting administration of the drug is of little value if any acute toxicity is dose-limiting. Furthermore, the reactive group is likely to react with plasma and tissue constituents, leading to a complicated pharmacokinetic profile. As described hereinbelow, a FAS screening assay was employed to screen for reversible antagonists of human FAS which may be useful in treating tumors or obesity, or preventing or inhibiting cell proliferation, e.g., endothelial cell proliferation, thereby inhibiting angiogeneisis.

Exemplary Pathogens with TE Containing Polypeptides

One unique approach toward generating anti-infectives, including drugs to combat Y. pestis, B. anthracis, Vibrio spp., Salmonella spp., and Listeria spp., is to ablate their ability to acquire iron from the host, which is essential for their survival. At physiologic pH, Fe3+ is insoluble at concentrations above 10⁻¹⁸ M. In humans, the concentration of free Fe3+ is maintained at less than 10⁻²⁴ M to prevent iron toxicity (Raymond et al., 2003), which necessitates an active acquisition pathway by pathogens. Many bacteria have evolved an elaborate system of iron acquisition and transport. A common component of these systems is a molecule called a siderophore, which binds tightly to iron and is released into the host where it chelates iron from host proteins and then delivers it to the bacteria for internalization and use.

Y. pestis is the causative agent of Bubonic plague, the most lethal disease pandemic in history. The Bubonic plague wiped out one quarter of the European population in the 14th century. It is estimated that 25 million people died of the plague within a 5 year time frame. Y. pestis synthesizes a siderophore called yersiniabactin (Ybt), which is essential for virulence of the pathogen in vivo. Two TEs are essential for synthesis of yersiniabactin. The C-terminal thioesterase domain of HMWP-1 releases the completed yersiniabactin molecule. Mutation of the active site serine of this enzyme prevents the synthesis of Ybt (Bobrov et al., 2002), establishing this domain of HMWP 1 as a valid drug target. The second thioesterase required for synthesis of Ybt is encoded by the YbtT gene. YbtT is not necessary for production of yersiniabactin in vitro, however, the deletion of this gene prevents synthesis of yersiniabactin in vivo, establishing it as a valid drug target (Geoffrey et al., 2000).

Moreover, yersiniabactin is believed to be a virulence factor for pathogenic extraintestinal strains of E. coli, and for strains of E. coli that cause persistent urinary tract infections in hospital patients (Schubert et al., 2002; Schubert et al., 2000; Schubert et al., 1998). Therefore, drugs targeting Ybt biosynthesis may be useful in treating these more common infections.

Like Y. pestis, the CDC lists B. anthracis as a Category A Critical Biological Agent. In October 2001, aerosolized B. anthracis disseminated to victims via the U.S. Postal system resulted in 22 anthrax cases with five deaths from inhalation. The World Health Organization estimated that 50 kg of aerosolized B. anthracis released by airplane over a centralized population of 500,000 could travel 20 km and kill up to 20% of the population (WHO, 1970). Like Y. pestis, B. anthracis produces two known siderophores, anthrachelin and anthrabactin (Cendrowski et al., 2004), which may require one or more TE containing polypeptides for synthesis.

Gram-positive Mycobacterium tuberculosis causes tuberculosis (TB), a chronic wasting disease characterized by fever, weight loss, and lung tissue destruction. One third of the world's population is infected with TB; one new infection occurs every second (WHO, 2004). It is estimated that 40 million people will die from TB over the next 25 years (WHO, 2001). Multi drug resistant tuberculosis (MDR) is especially prevalent in non-Westernized countries.

M. tuberculosis survival in the human host relies on lipid metabolism (Cole et al., 1998). Branched chain mycolic acids form a protective lipid cell barrier to antibiotics and chemotherapy drugs (Parish et al., 1997; Liu et al., 1999). In mycolic acid synthesis, a TE domain catalyzes release of long chain FA from a multifunctional FAS (FAS-I; similar to eukaryotic FAS) (Kolattukudy et al., 1997; Kinsella et al., 2003). A second, prokaryotic multi-enzyme FASII complex extends these FA precursors, and the final TE domain on this enzyme releases C56 chains (Quemard et al., 1995). Inactivation of the FASII TE enzyme induces Mycobacterium cell lysis making it a potential drug target (Vilcheze et al., 2000).

A third TE from Mycobacterium mediates a condensation reaction involved in the production of mycolic acid from C56 precursors (Portevin et al., 2004). Therefore, inhibition of any one of these mycobacterium TEs is a rational strategy for development of antituberculosis drugs.

Buruli ulcer, a severely deforming skin infection of tropical Africa and Asia, results from infection by Mycobacterium ulcerans, a microbe that is genetically similar to those responsible for tuberculosis and leprosy. A polyketide toxin produced by M. ulcerans, called mycolactone, is responsible for the skin lesions of Burili, and is one of a new class of virulence determinants. Three giant modular PKS enzymes are involved in the biosynthesis of mycolactone: MLSA1 (1.8 MDa) and MLSA2 (0.26 MDa) produce the 12-membered lactone core while its unsaturated triol side chain is assembled by MLSB (1.2 MDa) (Stinear et al., 2004). Interestingly, there are two TE domains that have identical sequence, but different function: one is responsible for cyclization of the core and one catalyzes release of the fatty acid side chain. The inhibition of mycolactone biosynthesis via selective antagonists of the mycolactone synthase TE domains provides an attractive approach for remediation of Buruli ulcers.

Infection with group A Streptococcus (GAS) S. pyogenes results in cellulitis, sepsis, necrotizing fasciitis, and sequelae such as acute rheumatic fever (Cunningham et al., 2000). “Flesh-eating bacteria” invade skin and destroy soft tissue and limbs (Stevens, 1999). Many strains have developed resistance to common antibiotics such as penicillin, macrolides (erythromycin, lincomycin), and fluoroquinolones. Comparative genomic analysis has located Streptococcal pathogenecity islands as regions coding for known virulence factors. These pathogenicity islands have been identified in streptococcus isolated from patients with toxic shock syndrome (Beres et al., 2002; Nakagawa et al., 2003), infected wounds (Ferretti et al., 2001), acute rheumatic fever (Jernigan et al., 2001), and pharyngitis (Banks et al., 2004). Within these pathogenicity islands are a series of TE domains that could serve as drug targets in the treatment of S. pyogenes.

Assays to Identify Select TE Antagonists

In general, compounds that inhibit the activity of a TE domain, e.g., one in a FAS, can be identified from libraries of natural, synthetic or semi-synthetic products or extracts according to methods known in the art. Such screening methods include but are not limited to serine hydrolase activity-profiling assays, [¹⁴C]-acetate incorporation assays, iron chelation assays (for pathogens), or mass spectrometry, e.g., to measure sideropheres or polyketide synthesis. Accordingly, virtually any number of chemical extracts or compounds can be screened.

Samples for use in the assay methods of the invention include any sample that can be tested for FAS or TE activity and/or that can be used to identify compounds that inhibit FAS or TE or a disease that involves or is associated with a FAS or other TE containing polypeptide. Examples include, but are not limited to: a sample from a patient or subject, such as a cell, tissue, or tumor sample; a cell (e.g., a prokaryotic or eukaryotic cell that expresses endogenous or recombinant FAS or other TE containing polypeptide); a lysate (or lysate fraction) or extract derived from a cell; or a molecule derived from a cell or cellular material, e.g., purified recombinant TE containing polypeptides such as fusion polypeptides.

For instance, recombinant fusions with TE domains are expressed, e.g., in prokaryotic systems such as E. coli or in eukaryotic systems such as baculovirus expression systems. In one embodiment, the TE domain is fused to a tag useful to identify or purify the fusion, e.g., a His tag, glutathione S-transferase (GST) or maltose binding protein (MBP). The tag may be at the N-terminus, C-terminus, or both. In one embodiment, a ACP may be part of the fusion.

In one embodiment, the TE domain is one from a polypeptide from a pathogen including, but not limited to, Escherichia coli O157:H7, Legionella pneumophila, Neisseria gonorrhoeae, Neisseria meningitides, Salmonella typhi, Salmonella typhimurium, Shigella, Vibrio cholerae, Yersinia pestis, Mycobacterium tuberculosis, Haemophilus influenzae, Chlamydia pneumoniae, Yersinia enterocolitica, Streptococcus pneumoniae, Mycobacterium leprae, and Bacillus anthracis. In one embodiment, the TE domain is from a TE containing polypeptide including, but not limited to, N-(5-amino-5-carboxypentanoyl)-L-cysteinyl-D-valine synthase, bacitracin synthetase 3, carboxylesterase bioH, enterobactin synthetase component F, carboxylesterase 2,3-hydroxydecanoyl-[acyl-carrier-protein] dehydratase, fatty acid synthase subunit beta, lovastatin nonaketide synthase, acyl transferase, phenylacetic acid degradation protein paaI, aflatoxin biosynthesis polyketide synthase, anguibactin biosynthesis thioesterase, sterigmatocystin biosynthesis polyketide synthase (PKS), thioesterase tesA, acyl-CoA thioesterase II, fatty acid synthase subunit TOXC, protein vd1D, Conidial yellow pigment biosynthesis PKS, acyl-CoA thioester hydrolase CT535, acyl-CoA thioester hydrolase CPn0654/CP0093/CPj0654/CpB0680, acyl-CoA thioester hydrolase TC0822, esterase ybdB, acyl-CoA thioester hydrolase ybgC, acyl-CoA thioester hydrolase yciA, esterase ydiI, polyketide synthase from Glomerella lagenarium, acyl-CoA thioesterase Tes2, Tes3, Tes 4 or Tes5, peroxisomal acyl-CoA thioesterase Tes1, PksA from Aspergillus sp. L, Aspergillus nomius or Aspergillus flavus, Type I PKS from Gibberella zeae, Gibberella moniliformis, Ceratocystis resinifera or Leptosphaeria maculans, peroxisomal acyl-coenzyme A thioester hydrolase, polyketide synthase from Botrytis cinerea, Aspergillus parasiticus, Aspergillus terreus, Aspergillus fumigatus, Bipolaris oryzae, Cercospora nicotianae or Cochliobolus heterostrophus, Nectria haematococca acyl-CoA thioesterase, acyl-CoA thioesterase II, palmitoyl-protein thioesterase, acyl-protein thioesterase-1, acyl-CoA thioesterase, e.g., acyl-CoA thioesterase II, 32.2 kDa salivary protein from Lutzomyia longipalpis, HMWP1 protein and Irp4 protein from Yersinia enterocolitica, pyochelin synthetase from Pseudomonas aeruginosa or TubF protein from Angiococcus disciformis.

In another embodiment, the TE domain is from a eukaryotic polypeptide, such as a mammalian FAS, a mammal including but not limited to a rodent, e.g., mouse, rat, rabbit, hamster, mink or guinea pig, bovine, ovine, caprine, swine, equine, feline, canine, human or non-human primate.

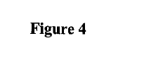

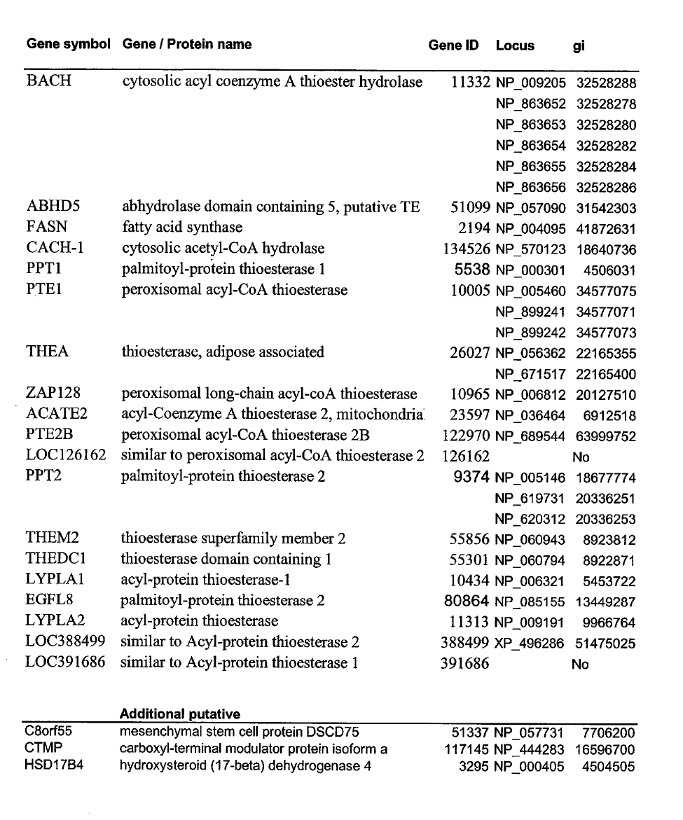

To identify TE antagonists specific for one or more pathogens, human TE containing polypeptides may be used in a counter screen. FIG. 4 provides an exemplary list of human TE containing polypeptides. Particular human TE containing polypeptides useful for counter screening are mitochondrial, peroxisomal, and cytosolic TEs (MTE, PTE, CTE), which regulate lipid metabolism by modulating cellular levels of free fatty acid, acyl-CoA, and CoASH and may be involved in cell signaling. CTE-II, also known as human brain acyl-CoA hydrolase (BACH), is unique in that there are isoforms with localization signals that direct the expression of BACH to the cytosol, nucleus, or mitochondria (Yamada et al., 2002; Yamada et al., 1999). Other human TE containing polypeptides that may be employed in a counter screen include, but are not limited to, palmitoyl-protein thioesterases (PPT) (PPT-1 is highly expressed in human brain tissue, and mutations in the gene encoding PPT-1 lead to the neuronal ceroid lipfuscinosis (NCL) disease), brown fat inducible thioesterase (BFIT) (BFIT may regulate lipid metabolism by controlling levels of available cellular acyl-CoA and terminating de novo fatty acid synthesis; Adams et al., 2001), CGI58 protein (diagnosis of Chanarin-Dorfinan syndrome (ADS) has been linked to mutations in the gene encoding CG158 proteins; such as Lefevre et al., 2001), and a palmitoyl thioesterase (PTE) linked to AIDS.

In another embodiment, TE antagonists specific for human FAS are identified and those compounds may be useful as antineoplastics or antiobesity drugs (see Example I) or for other disorders. In addition, antagonists of any other human TE containing polypeptide may be identified by assays described herein or others known to the art.

In one embodiment, the antagonists identified in the screening assay are reversible antagonists. In one embodiment, the antagonists identified in the screening assay are partial non-competitive inhibitors. In another embodiment, the antagonists identified by the method are non-competitive inhibitors.

Definitions

Unless stated otherwise, the following terms and phrases as used herein are intended to have the following meanings:

When trade names are used herein, applicants intend to independently include the trade name product and the active pharmaceutical ingredient(s) of the trade name product.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

One diastereomer of a compound disclosed herein may display superior activity compared with the other. When required, separation of the racemic material can be achieved by HPLC using a chiral column or by a resolution using a resolving agent such as camphonic chloride as in Tucker et al. (1994). A chiral compound of Formula I may also be directly synthesized using a chiral catalyst or a chiral ligand, e.g., Huffinan et al., (1995).

“Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy, as described for example by Chou et al. (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy. When a substituent is keto (i.e., ═O) or thioxo (i.e., ═S) group, then 2 hydrogens on the atom are replaced.

“Interrupted” is intended to indicate that in between two or more adjacent carbon atoms, and the hydrogen atoms to which they are attached (e.g., methyl (CH₃), methylene (CH₂) or methine (CH)), indicated in the expression using “interrupted” is inserted with a selection from the indicated group(s), provided that the each of the indicated atoms' normal valency is not exceeded, and that the interruption results in a stable compound. Such suitable indicated groups include, e.g., with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO) or sulfonyl (SO₂).

Specific and preferred values listed below for radicals, substituents, and ranges, are for illustration only; they do not exclude other defined values or other values within defined ranges for the radicals and substituents

“Alkyl” refers to a C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH3)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The alkyl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy. The alkyl can optionally be interrupted with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO) or sulfonyl (SO₂). Additionally, the alkyl can optionally be at least partially unsaturated, thereby providing an alkenyl.

“Alkenyl” refers to a C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

The alkenyl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy. Additionally, the alkenyl can optionally be interrupted with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO) or sulfonyl (SO₂).

“Alkylidenyl” refers to a C₁-C₁₈ hydrocarbon containing normal, secondary, tertiary or cyclic carbon atoms. Examples are methylidenyl (═CH₂), ethylidenyl (═CHCH₃), 1-propylidenyl (═CHCH₂CH₃), 2-propylidenyl (═C(CH₃)₂), 1-butylidenyl (═CHCH₂CH₂CH₃), 2-methyl-1-propylidenyl (═CHCH(CH₃)₂), 2-butylidenyl (═C(CH₃)CH₂CH₃), 1-pentyl (═CHCH₂CH₂CH₂CH₃), 2-pentylidenyl (═C(CH₃)CH₂CH₂CH₃), 3-pentylidenyl (═C(CH₂CH₃)₂), 3-methyl-2-butylidenyl (═C(CH₃)CH(CH₃)₂), 3-methyl-1-butylidenyl (═CHCH₂CH(CH₃)₂), 2-methyl-1-butylidenyl (═CHCH(CH₃)CH₂CH₃), 1-hexylidenyl (═CHCH₂CH₂CH₂CH₂CH₃), 2-hexylidenyl (═C(CH₃)CH₂CH₂CH₂CH₃), 3-hexylidenyl (═C(CH₂CH₃)(CH₂CH₂CH₃)), 3-methyl-2-pentylidenyl (═C(CH₃)CH(CH₃)CH₂CH₃) 4-methyl-2-pentylidenyl (═C(CH₃)CH₂CH(CH₃)₂), 2-methyl-3-pentylidenyl (═C(CH₂CH₃)CH(CH₃)₂), and 3,3-dimethyl-2-butylidenyl (═C(CH₃)C(CH₃)₃.

The alkylidenyl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy. Additionally, the alkylidenyl can optionally be interrupted with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO) or sulfonyl (SO₂).

“Alkenylidenyl” refers to a C₂-C₁₈ hydrocarbon containing normal, secondary, tertiary or cyclic carbon atoms with at least one site of unsaturation, i.e. a carbon-carbon, sp² double bond. Examples include, but are not limited to: allylidenyl (═CHCH═CH₂), and 5-hexenylidenyl (═CHCH₂CH₂CH₂CH═CH₂).

The alkenylidenyl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy. Additionally, the alkenylidenyl can optionally be interrupted with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO) or sulfonyl (SO₂).

“Alkylene” refers to a saturated, branched or straight chain or cyclic hydrocarbon radical of 1-18 carbon atoms, and having two monovalent radical centers derived by the removal of two hydrogen atoms from the same or different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The alkylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy. Additionally, the alkylene can optionally be interrupted with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO) or sulfonyl (SO₂). Moreover, the alkylene can optionally be at least partially unsaturated, thereby providing an alkenylene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

The alkenylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy. Additionally, The alkenylene can optionally be interrupted with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO) or sulfonyl (SO₂).

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment of the invention, the nitrogen heterocycle can be 3-methyl-5,6-dihydro-4H-pyrazino[3,2,1-jk]carbazol-3-ium iodide.

Another class of heterocyclics is known as “crown compounds” which refers to a specific class of heterocyclic compounds having one or more repeating units of the formula [—(CH₂—)ₐA-] where a is equal to or greater than 2, and A at each separate occurrence can be O, N, S or P. Examples of crown compounds include, by way of example only, [—(CH₂)₃—NH—]₃, [—((CH₂)₂—)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “acyloxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “imino” refers to —C═NH. The imino can optionally be substituted with one or more alkyl, alkenyl, alkoxy, aryl, heteroaryl, heterocycle or cycloalkyl.

The term “nitro” refers to —NO₂.

The term “trifluoromethyl” refers to —CF₃.

The term “trifluoromethoxy” refers to —OCF₃.

The term “cyano” refers to —CN.

The term “hydroxy” or “hydroxyl” refers to —OH.

The term “oxy” refers to —O—.

The term “thio” refers to —S—.

The term “thioxo” refers to (═S).

The term “keto” refers to (═O).

The term “isocyannato” refers to —NC.

The chemical structures of additional groups are shown in the table below.

| Name | Structure |
|---|---|
| acetamido | 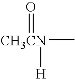 |
| Acetoxy | 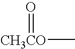 |
| Acetyl | 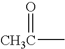 |
| benzamido | 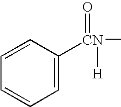 |
| benzenesulfinyl | 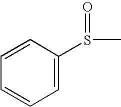 |
| benzenesulfonamido | 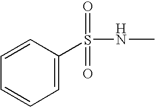 |
| benzenesulfonyl | 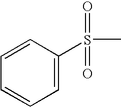 |
| benzoyl | 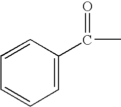 |
| benzoylamino | 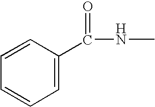 |
| benzoyloxy | 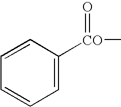 |
| Benzyl | 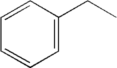 |
| benzyloxy | 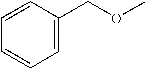 |
| benzyloxycarbonyl | 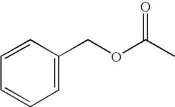 |
| benzylthio | 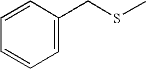 |
| carbamoyl | 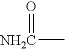 |
| sulfamoyl | 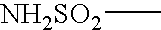 |
| sulfinamoyl | 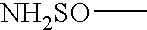 |
| Sulfino | 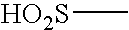 |
| Sulfo | 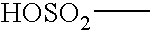 |
| sulfoamino | 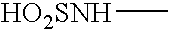 |
| thiosulfo | 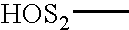 |

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the invention. One of ordinary skill in the art of medicinal and organic chemistry understands the versatility of such substituents. To the degree that recursive substituents are present in an claim of the invention, the total number will be determined as set forth above.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

Pro-drugs include hydroxyl and amino derivatives well-known to practitioners of the art, such as, for example, esters prepared by reaction of the parent hydroxyl compound with a suitable carboxylic acid, or amides prepared by reaction of the parent amino compound with a suitable carboxylic acid. Simple aliphatic or aromatic esters derived from hydroxyl groups pendent on the compounds employed in this invention are preferred pro-drugs. In some cases it may be desirable to prepare double ester type pro-drugs such as (acyloxy) alkyl esters or ((alkoxycarbonyl)oxy)alkyl esters. Specific suitable esters as pro-drugs include methyl, ethyl, propyl, isopropyl, n-butyl, isobutyl, tert-butyl, and morpholinoethyl.

Hydrolysis in Drug and Pro-drug Metabolism: Chemistry Biochemistry, and Enzymology (2003), provides a comprehensive review of metabolic reactions and enzymes involved in the hydrolysis of drugs and pro-drugs. The text also describes the significance of biotransformation and discusses the physiological roles of hydrolytic enzymes, hydrolysis of amides, and the hydrolysis of lactams. Additional references useful in designing pro-drugs employed in the present invention include, e.g., Biological Approaches to the Controlled Delivery of Drugs (1988); Design of Biobiological agent Properties through Pro-drugs and Analogs (1977); Pro-drugs: Topical and Ocular Drug Delivery (1992); Enzyme-Pro-drug Strategies for Cancer Therapy (1999); Design of Pro-drugs (1986); Textbook of Drug Design and Development (1991); Conversion of Non-Toxic Pro-drugs to Active, Anti-Neoplastic Drugs Selectively in Breast Cancer Metastases (2000); and Marine lipids for prodrugs, of compounds and other biological agent applications (2000).

Pro-drugs employed in the present invention can include any suitable functional group that can be chemically or metabolically cleaved by solvolysis or under physiological conditions to provide the biologically active compound. Suitable functional groups include, e.g., carboxylic esters, amides, and thioesters. Depending on the reactive functional group(s) of the biologically active compound, a corresponding functional group of a suitable linker precursor can be selected from the following table, to provide, e.g., an ester linkage, thioester linkage, or amide linkage in the pro-drug.

| Functional Group on | | |
|---|---|---|
| Biologically Active | Functional Group on | Resulting Linkage |
| Compound | Linker Precursor | in Pro-drug |
| —COOH | —OH | Ester |
| —COOH | —NH₂ | Amide |
| —COOH | —SH | Thioester |
| —OH | —COOH | Carboxylic Ester |
| —SH | —COOH | Thioester |
| —NH₂ | —COOH | Amide |
| —OH | —OP(═O)(OH)₂ | Phosphoric Acid Ester |
| —OH | —OP(═O)(OR)₂ | Phosphoric Acid Ester |
| —OH | —SO₂OH | Sulphonic Acid Ester |

Linker Precursor and Linking Group

A biologically active compound can be linked to a suitable linker precursor to provide the pro-drug. As shown above, the reactive functional groups present on the biologically active compound will typically influence the functional groups that need to be present on the linker precursor. The nature of the linker precursor is not critical, provided the pro-drug employed in the present invention possesses acceptable mechanical properties and release kinetics for the selected therapeutic application. The linker precursor is typically a divalent organic radical having a molecular weight of from about 25 daltons to about 400 daltons. More preferably, the linker precursor has a molecular weight of from about 40 daltons to about 200 daltons.

The resulting linking group, present on the pro-drug, may be biologically inactive, or may itself possess biological activity. The linking group can also include other functional groups (including hydroxy groups, mercapto groups, amine groups, carboxylic acids, as well as others) that can be used to modify the properties of the pro-drug (e.g., for appending other molecules) to the pro-drug, for changing the solubility of the pro-drug, or for effecting the biodistribution of the pro-drug).

Specifically, the linking group can be a divalent, branched or unbranched, saturated or unsaturated, hydrocarbon chain, having from 1 to 50 carbon atoms, wherein one or more (e.g., 1, 2, 3, or 4) of the carbon atoms is optionally interrupted with, e.g., one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO), sulfonyl (SO₂) or (—NR—), wherein R can be hydrogen, alkyl, cycloalkyl alkyl, or aryl alkyl.

The hydrocarbon chain of the linking group is optionally substituted on carbon with one or more (e.g., 1, 2, 3, or 4) substituents selected from the group of alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

Methods of Making the Compounds of the Invention.

The compounds of the present invention can be prepared by any of the applicable techniques of organic synthesis. Many such techniques are well known in the art. However, many of the known techniques are elaborated in Compendium of Organic Synthetic Methods (Vol. 1, 1971; Vol. 2, 1974; Vol. 3, 1977; Vol. 4, 1980; Vol. 5, 1984; and Vol. 6 as well as March in Advanced Organic Chemistry (1985); Comprehensive Organic Synthesis. Selectivity, Strategy & Efficiency in Modern Organic Chemistry. In 9 Volumes (1993); Advanced Organic Chemistry Part B: Reactions and Synthesis, Second Edition (1983); Advanced Organic Chemistry, Reactions, Mechanisms, and Structure, Second Edition (1977); Protecting Groups in Organic Synthesis, Second Edition; and Comprehensive Organic Transformations (1999).

Compounds of Formula (I)

The present invention provides a compound of formula (I): 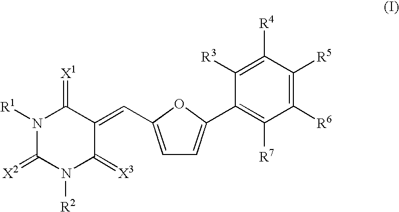
wherein,

X¹ is O, S or NOH;

X² is O, S or NOH;

X³ is O, S or NOH;

R¹ is H, alkyl, alkenyl, haloalkyl, hydroxyalkyl, aryl, heteroaryl, heterocycle, or cycloalkyl;

R² is H, alkyl, alkenyl, haloalkyl, hydroxyalkyl, aryl, heteroaryl, heterocycle, or cycloalkyl;

R³ is alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁴ is alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁵ is alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁶ is alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; and

R⁷ is alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl.

Compounds of Formula (II)

The present invention also provides a compound of formula (II): 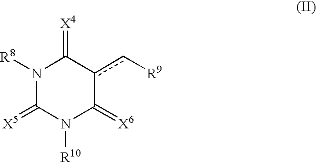
wherein,

X⁴ is O, S or NOH;

X⁵ is O, S or NOH;

X⁶ is O, S or NOH;

R⁸ is H, alkyl, alkenyl, haloalkyl, hydroxyalkyl, aryl, heteroaryl, heterocycle, or cycloalkyl;

R⁹ is alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R¹⁰ is H, alkyl, alkenyl, haloalkyl, hydroxyalkyl, aryl, heteroaryl, heterocycle, or cycloalkyl; and

the optional double bond is absent or present.

Compounds of Formula (III)

The present invention also provides a compound of formula (III): 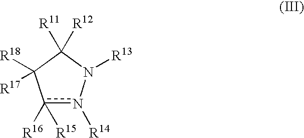
wherein,

R¹¹ is alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R¹¹ and R¹² together are oxo (═O), thixo (═S) or oxime (═NOH);

R¹² is alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R¹¹ and R¹² together are oxo (═O), thixo (═S) or oxime (═NOH);

R¹³ is H, alkyl, alkenyl, haloalkyl, hydroxyalkyl, aryl, heteroaryl, heterocycle, or cycloalkyl;

R¹⁴ is absent, H, alkyl, alkenyl, haloalkyl, hydroxyalkyl, aryl, heteroaryl, heterocycle, or cycloalkyl;

R¹⁵ is absent, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R¹⁵ and R¹⁶ together are oxo (═O), thixo (═O) or oxime (═NOH);

R¹⁶ is absent, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x} , wherein each R^{x} and R^{y} is independently H, alkyl, alkenyl, aryl, heteroaryl, heterocycle, cycloalkyl or hydroxyl; or R¹⁵ and R¹⁶ together are oxo (═O), thixo (═O) or oxime (═NOH);

R¹⁷ is alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x} , wherein each R^{x} and R^{y} is independently H, alkyl, alkenyl, aryl, heteroaryl, heterocycle, cycloalkyl or hydroxyl; or R¹⁷ and R¹⁸ together are alkylidenyl or alkenylidenyl;

R¹⁸ is alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R¹⁷ and R¹⁸ together are alkylidenyl or alkenylidenyl; and

the optional double bond is absent or present.

Compounds of Formula (IV)

The present invention also provides a compound of formula (IV): 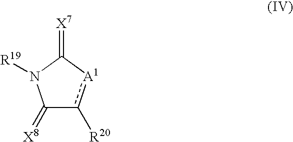
wherein,

X⁷ is O, S or NOH;

X⁸ is O, S or NOH;

A¹ is S, CH, CH₂, N, NH, NR^{x}, CR^{x}or CHR^{x} wherein R^{x} is independently H, alkyl, alkenyl, aryl, heteroaryl, heterocycle, cycloalkyl or hydroxyl;

R¹⁹ is H, alkyl, alkenyl, haloalkyl, hydroxyalkyl, aryl, heteroaryl, heterocycle, or cycloalkyl;

R²⁰ is SR^{z}, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl, wherein R^{z} is alkyl, alkenyl, aryl, heteroaryl, heterocycle, cycloalkyl, amino or imino; and

the optional bond is absent or present.

Compounds of Formula (V)

The present invention also provides a compound of formula (V): 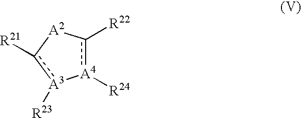
wherein,

A² is O, CH₂, NH, NR^{x}, or CHR^{x} wherein R^{x} is independently H, alkyl, alkenyl, aryl, heteroaryl, heterocycle, cycloalkyl or hydroxyl;

A³ is N, C, CH, or CR^{x} wherein R^{x} is independently H, alkyl, alkenyl, aryl, heteroaryl, heterocycle, cycloalkyl or hydroxyl;

A⁴ is N, C, CH, or CR^{x} wherein R^{x} is independently H, alkyl, alkenyl, aryl, heteroaryl, heterocycle, cycloalkyl or hydroxyl;

R²¹ is H, alkyl, alkenyl, , alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R²² is SR^{z}, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl, wherein R^{z} is alkyl, alkenyl, aryl, heteroaryl, heterocycle, cycloalkyl, amino or imino;

R²³ is absent, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R²⁴ is absent, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; and

each of the optional bonds are independently absent or present.

Compounds of Formula (VI)

The present invention also provides a compound of formula (VI): 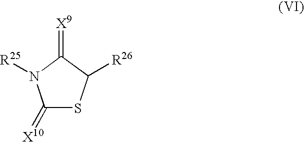
wherein,

X⁹ is O, S or NOH;

X¹⁰ is O, S or NOH;

R²⁵ is H, alkyl, alkenyl, haloalkyl, hydroxyalkyl, aryl, heteroaryl, heterocycle, or cycloalkyl; and

R²⁶ is H, alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl.

Compounds of Formula (VII)

The present invention also provides a compound of formula (VII): 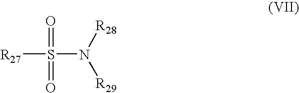
wherein,

R²⁷ is H, alkyl, alkenyl, alkoxy, haloalkyl, hydroxy, hydroxyalkyl, aryl, heteroaryl, heterocycle, or cycloalkyl;

R²⁸ is H, alkyl, alkenyl, alkylidenyl, alkenylidenyl, alkoxy, haloalkyl, hydroxyalkyl, aryl, heteroaryl, heterocycle, cycloalkyl, or R²⁸ and R²⁹ together are alkyl, alkenyl, alkylidenyl, alkenylidenyl, alkoxy, haloalkyl, hydroxy, hydroxyalkyl, aryl, heteroaryl, heterocycle, cycloalkyl, arylidenyl, heteroarylidenyl, heterocyclidenyl, cycloalkylidenyl; and

R²⁹ is H, alkyl, alkenyl, alkylidenyl, alkenylidenyl, alkoxy, haloalkyl, hydroxyalkyl, aryl, heteroaryl, heterocycle, cycloalkyl, or R²⁸ and R²⁹ together are alkyl, alkenyl, alkylidenyl, alkenylidenyl, alkoxy, haloalkyl, hydroxy, hydroxyalkyl, aryl, heteroaryl, heterocycle, cycloalkyl, arylidenyl, heteroarylidenyl, heterocyclidenyl, cycloalkylidenyl.

Compounds of Formula (VIII)

The present invention also provides a compound of formula (VIII): 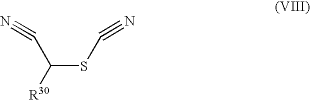
wherein,

R³⁰ is H, alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl.

Compounds of Formula (IX)

The present invention also provides a compound of formula (IX): 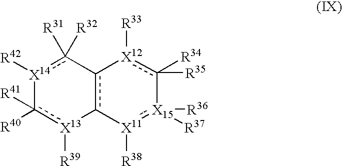
wherein,

X¹¹ is C, CH, N or CR^{x} wherein R^{x} is independently H, alkyl, alkenyl, aryl, heteroaryl, heterocycle, cycloalkyl or hydroxyl;

X¹² is C, CH, N or CR^{x} wherein R^{x} is independently H, alkyl, alkenyl, aryl, heteroaryl, heterocycle, cycloalkyl or hydroxyl;

X¹³ is C, CH, N or CR^{x} wherein R^{x} is independently H, alkyl, alkenyl, aryl, heteroaryl, heterocycle, cycloalkyl or hydroxyl;

X¹⁴ is C, CH, N or CR^{x} wherein R^{x} is independently H, alkyl, alkenyl, aryl, heteroaryl, heterocycle, cycloalkyl or hydroxyl;

X¹⁵ is C, CH, N or CR^{x} wherein R^{x} is independently H, alkyl, alkenyl, aryl, heteroaryl, heterocycle, cycloalkyl or hydroxyl;

R³¹ is absent, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R³¹ and R³² together are oxo (═O), thioxo (═S) or oxime (═NOH);

R³² is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R³¹ and R³² together are oxo (═O), thioxo (═S) or oxime (═NOH);

R³³ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R³³ and R³⁴ together form aryl, heteroaryl, heterocycle or cycloalkyl;

R³⁴ is absent, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R³³ and R³⁴ together form aryl, heteroaryl, heterocycle or cycloalkyl;

R³⁵ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, cyano, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R³⁶ is absent, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R³⁶ and R³⁷ together are oxo (═O), thioxo (═S) or oxime (═NOH);

R³⁷ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R³⁶ and R³⁷ together are oxo (═O), thioxo (═S) or oxime (═NOH);

R³⁸ is absent, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R³⁹ is SR^{z}, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl, wherein R^{z} is alkyl, alkenyl, aryl, heteroaryl, heterocycle, cycloalkyl, amino or imino;

R⁴⁰ is absent, H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R⁴⁰ and R⁴¹ together are oxo (═O), thioxo (═S) or oxime (═NOH);

R⁴¹ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; or R⁴⁰ and R⁴¹ together are oxo (═O), thioxo (═S) or oxime (═NOH);

R⁴² is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; and

each of the optional bonds are independently absent or present.

Compounds of Formula (X)

The present invention also provides a compound of formula (X): 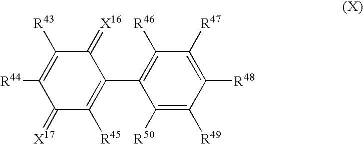
wherein,

X¹⁶ is O, S or NOH;

X¹⁷ is O, S or NOH;

R⁴³ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁴⁴ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁴⁵ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁴⁶ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁴⁷ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁴⁸ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁴⁹ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; and

R⁵⁰ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl.

Compounds of Formula (XI)

The present invention also provides a compound of formula (XI): 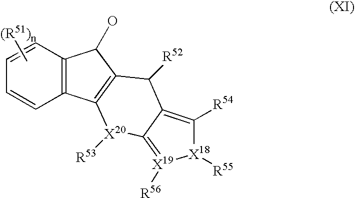
wherein,

X¹⁸ is N, CH or CR^{x} wherein R^{x} is H, alkyl, alkenyl, aryl, heteroaryl, heterocycle, cycloalkyl or hydroxyl;

X¹⁹ is N or C;

X²⁰ is N, CH or CR^{x} wherein R^{x} is H, alkyl, alkenyl, aryl, heteroaryl, heterocycle, cycloalkyl or hydroxyl;

R⁵¹ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁵² is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁵³ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁵⁴ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁵⁵ is H, alkyl, alkenyl, alkylidenyl, alkenylidenyl, alkoxy, haloalkyl, hydroxyalkyl, aryl, heteroaryl, heterocycle, cycloalkyl;

R⁵⁶ is absent, H, alkyl, alkenyl, alkoxy, haloalkyl, hydroxyalkyl, aryl, heteroaryl, heterocycle, cycloalkyl; and

n=0-4.

Compounds of Formula (XII)

The present invention also provides a compound of formula (XII): 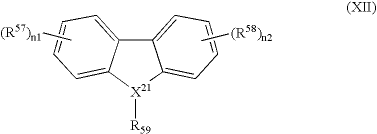
wherein,

X²¹ is N, CH or CR^{x} wherein R^{x} is H, alkyl, alkenyl, aryl, heteroaryl, heterocycle, cycloalkyl or hydroxyl;

R⁵⁷ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁵⁸ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁵⁹ is H, alkyl, alkenyl, alkylidenyl, alkenylidenyl, alkoxy, haloalkyl, hydroxyalkyl, aryl, heteroaryl, heterocycle, or cycloalkyl;

n1 is 0-4; and

n2 is 0-4.

Compounds of Formula (XIII)

The present invention also provides a compound of formula (XIII): 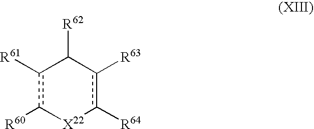
wherein,

X²² is NH, NR^{x}, CHR^{x} or CR^{x}R^{x} wherein each R^{x} is independently H, alkyl, alkenyl, aryl, heteroaryl, heterocycle, cycloalkyl or hydroxyl;

R⁶⁰ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁶¹ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁶² is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl;

R⁶³ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x} , wherein each R^{x} and R^{y} is independently H, alkyl, alkenyl, aryl, heteroaryl, heterocycle, cycloalkyl or hydroxyl;

R⁶⁴ is H, alkyl, alkenyl, alkoxy, halo, haloalkyl, hydroxy, hydroxyalkyl, aryl, heteroaryl, heterocycle, cycloalkyl, alkanoyl, alkoxycarbonyl, amino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} or COOR^{x}, wherein each R^{x} and R^{y} is independently H, alkyl, alkenyl, aryl, heteroaryl, heterocycle, cycloalkyl or hydroxyl; and

each of the optional bonds are independently absent or present.

Specific Ranges, Values, and Embodiments

Obviously, numerous modifications and variations of the present invention are possible in light of the above teachings. It is therefore to be understood that within the scope of the appended claims, the invention may be practiced otherwise than as specifically described herein.

Specific ranges, values, and embodiments provided below are for illustration purposes only and do not otherwise limit the scope of the invention, as defined by the claims.

For the compounds of formula (I):

A specific value for X¹ is O.

A specific value for X² is S. Another specific value for X² is O.

A specific value for X³ is O.

A specific value for R¹ is H.

A specific value for R² is H. Another specific value for R² is alkyl. Another specific value for R² is methyl.

A specific value for R³ is halo. Another specific value for R³ is nitro. Another specific value for R³ is hydroxyl. Another specific value for R³ is H. Another specific value for R³ is carboxylic (CO₂H).

A specific value for R⁴ is H.

A specific value for R⁵ is H. Another specific value for R⁵ is nitro. Another specific value for R⁵ is alkoxy. Another specific value for R⁵ is methoxy. Another specific value for R⁵ is alkyl. Another specific value for R⁵ is methyl. Another specific value for R⁵ is carboxylic (CO₂H).

A specific value for R⁶ is H. Another specific value for R⁶ is alkyl. Another specific value for R⁶ is methyl. Another specific value for R⁶ is nitro.

A specific value for R⁷ is H.

For the compounds of formula (II):

A specific value for X⁴ is O.

A specific value for X⁵ is O. Another specific value for X⁵ is S.

A specific value for X⁶ is O.

A specific value for R⁸ is H. Another specific value for R⁸ is alkyl. Another specific value for R⁸ is methyl.

A specific value for R⁹ is alkenyl. Another specific value for R⁹ is CH₂CH═CH-Ph. Another specific value for R⁹ is CH₂CH═CH-(o-NO₂)Ph. Another specific value for R⁹ is CH═CH(o-NO₂)Ph. Another specific value for R⁹ is alkyl. Another specific value for R⁹ is methyl. Another specific value for R⁹ is CH₂-(p-N(CH₃)₂)Ph or 4-(N,N-dimethylbenzenamine). Another specific value for R⁹ is CH₂-(p-OCH₂CH₃)Ph. Another specific value for R⁹ is CH₂CH₂Ph. Another specific value for R⁹ is imino. Another specific value for R⁹ is NH-(o-CH₃)Ph. Another specific value for R⁹ is aryl. Another specific value for R⁹ is heterocycle. Another specific value for R⁹ is 2-vinylfuran.

A specific value for R¹⁰ is aryl. Another specific value for R¹⁰ is 1,3-di-OCH₃-Ph. Another specific value for R¹⁰ is phenyl (Ph). Another specific value for R¹⁰ is (m-OCH₃)-Ph. Another specific value for R¹⁰ is o-fluorophenyl. Another specific value for R¹⁰ is (p-OCH₂CH₃)-Ph. Another specific value for R¹⁰ is (m-CH₃)-Ph. Another specific value for R¹⁰ is 2,5-di-OCH₃(Ph). Another specific value for R¹⁰ is (o-OCH₃)Ph. Another specific value for R¹⁰ is (p-Cl)Ph. Another specific value for R¹⁰ is alkyl. Another specific value for R¹⁰ is ethyl.

For the compounds of formula (III):

A specific value for R¹¹ is that R¹¹ and R¹² together are oxo (═O).

A specific value for R¹² is that R¹¹ and R¹² together are oxo (═O).

A specific value for R¹³ is H. Another specific value for R¹³ is heterocycle. Another specific value for R¹³ is 1-(4-phenylthiazol). Another specific value for R¹³ is aryl. Another specific value for R¹³ is 3,4-dichlorophenyl. Another specific value for R¹³ is m-bromophenyl. Another specific value for R¹³ is Ph. Another specific value for R¹³ is that R¹³ is absent.

A specific value for R¹⁴ is H. Another specific value for R¹⁴ is heterocycle. Another specific value for R¹⁴ is 2-(4-phenylthiazole). Another specific value for R¹⁴ is aryl. Another specific value for R¹⁴ is 3,4-di Cl-Ph. Another specific value for R¹⁴ is m-Br-Ph. Another specific value for R¹⁴is Ph. Another specific value for R¹⁴ is that R¹⁴ is absent.

A specific value for R¹⁵ is that R¹⁵ is absent. Another specific value for R¹⁵ is alkyl. Another specific value for R¹⁵ is methyl. Another specific value for R¹⁵ is hydroxyl. Another specific value for R¹⁵ is that R¹⁵ and R¹⁶ together are oxo (═O).

A specific value for R¹⁶ is that R¹⁶ is absent. Another specific value for R¹⁶ is alkyl. Another specific value for R¹⁶ is methyl. Another specific value for R¹⁶ is hydroxyl. Another specific value for R¹⁶ is that R¹⁵ and R¹⁶ together are oxo (═O).

A specific value for R¹⁷ is R¹⁷ and R¹⁸ together are alkylidenyl. Another specific value for R¹⁷ is R¹⁷ and R¹⁸ together are ═CH-p-phenol. Another specific value for R¹⁷ is R¹⁷ and R¹⁸ together are ═CH-p-Cl-Ph. Another specific value for R¹⁷ is R¹⁷ and R¹⁸ together are ═CH-(2-OCH₃-5-Cl)-Ph. Another specific value for R¹⁷ is R¹⁷ and R¹⁸ together are ═CH-(2,4-di-CH-5-NO₂-Ph). Another specific value for R¹⁷ is R¹⁷ and R¹⁸ together are ═CH-3-(indolin-2-one). Another specific value for R¹⁷ is R¹⁷ and R¹⁸ together are 4-(1-phenylpyrazolidine-3,5-dione).

A specific value for R¹⁸ is R¹⁷ and R¹⁸ together are alkylidenyl. Another specific value for R¹⁸ is R¹⁷ and R¹⁸ together are ═CH-p-phenol. Another specific value for R¹⁸ is R¹⁷ and R¹⁸ together are ═CH-p-Cl-Ph. Another specific value for R¹⁸ is R¹⁷ and R¹⁸ together are ═CH-(2-OCH₃-5-Cl)-Ph. Another specific value for R¹⁸ is R¹⁷ and R¹⁸ together are ═CH-(2,4-di-Cl-5-NO₂-Ph). Another specific value for R¹⁸ is R¹⁷ and R¹⁸ together are ═CH-3-(indolin-2-one). Another specific value for R¹⁸ is R¹⁷ and R¹⁸ together are 4-(1-phenylpyrazolidine-3,5-dione).

For the compounds of formula (IV):

A specific value for X⁷ is O. Another specific value for X⁷ is S.

A specific value for X⁸ is O.

A specific value for A¹ is (CH)j wherein j is 1-3. Another specific value for A¹ is CH. Another specific value for A¹ is S.

A specific value for R¹⁹ is aryl. Another specific value for R¹⁹ is 2-(1H-pyrrole-2,5-dione) phenyl. Another specific value for R¹⁹ is 1-(4-(difluoromethylthio)phenyl). Another specific value for R¹⁹ is 1-(2-bromo-4-methylphenyl). Another specific value for R¹⁹ is 1-(4-phenylethanone). Another specific value for R¹⁹ is 4-methylbenzoate. Another specific value for R¹⁹ is 1-(2-(trifluoromethylthio)phenyl). Another specific value for R¹⁹ is (E)-1-(2-(4-((imino)methyl)phenoxy)ethoxy)-3-methylbenzene. Another specific value for R¹⁹ is 1-(4-( N,N-dimethylbenzeneamine)). Another specific value for R¹⁹ is 1-(4-methoxyphenyl).

A specific value for R²⁰ is H. Another specific value for R²⁰ is an N,N′-disubstituted carbamimidothioate. Another specific value for R²⁰ is (E)-N-4-chlorobenzyl-N′-phenylcarbamimidothioate.

For the compounds of formula (V):

A specific value for A² is O.

A specific value for A³ is C. Another specific value for A³ is N. Another specific value for A³ is CH.

A specific value for A⁴ is C. Another specific value for A⁴ is N. Another specific value for A⁴ is CH.

A specific value for R²¹ is alkylidenyl. Another specific value for R²¹ is (E)-5-(methylene)-3-methyl-2-thioxothiazolidin-4-one. Another specific value for R²¹ is (Z)-5-(methylene)thiazolidine-2,4-dione. Another specific value for R²¹ is (E)-2-cyano-3-(2,4-dichlorophenyl)-N-(methyl)acrylamide. Another specific value for R²¹ is H. Another specific value for R²¹ is aryl. Another specific value for R²¹ is 1-(4-hydroxy-3-benzoic acid). Another specific value for R²¹ is 1-(3-F-Ph). Another specific value for R²¹ is 1-(3-NO₂-Ph). Another specific value for R²¹ is SR^{z}, wherein R^{z} is aryl. Another specific value for R²¹ is (4-chlorophenyl)sulfane.

A specific value for R²² is alkylidenyl. Another specific value for R²² is (E)-5-(methylene)-3-methyl-2-thioxothiazolidin-4-one. Another specific value for R²² is (Z)-5-(methylene)thiazolidine-2,4-dione. Another specific value for R²² is (E)-2-cyano-3-(2,4-dichlorophenyl)-N-(methyl)acrylamide. Another specific value for R²² is H. Another specific value for R²² is aryl. Another specific value for R²² is 1-(4-hydroxy-3-benzoic acid). Another specific value for R²² is 1-(3-F-Ph). Another specific value for R²² is 1-(3-NO₂-Ph). Another specific value for R²² is SR^{z}, wherein R^{z} is aryl. Another specific value for R²² is (4-chlorophenyl)sulfane.

A specific value for R²³ is H. A specific value for R²³ is that R²³ is absent.

A specific value for R²⁴ is H. A specific value for R²⁴ is that R²⁴ is absent.

For the compounds of formula (VI):

A specific value for X⁹ is O.

A specific value for X¹⁰ is S.

A specific value for R²⁵ is alkyl. Another specific value for R²⁵ is methyl.

Another specific value for R²⁵ is alkenyl. Another specific value for R²⁵ is CH₂CH═CH₂.

A specific value for R²⁶ is alkylidenyl. Another specific value for R²⁶ is 1-(3-benzyloxy)-vinylbenzyl. Another specific value for R²⁶ is 1-(4-vinylbenzoate).

For the compounds of formula (VII):

A specific value for R²⁷ is aryl. Another specific value for R²⁷ is p-Cl-Ph. Another specific value for R²⁷ is p-F-Ph. Another specific value for R²⁷ is p-Et-Ph.

A specific value for R²⁸ is H. Another specific value for R²⁸ is R²⁸ and R²⁹ together are cycloalkylidenyl. Another specific value for R²⁸ is R²⁸ and R²⁹ together are 2,3,5-trichloro-4-cyclohexylidene-2,5-dienone. Another specific value for R²⁸ is R²⁸ and R²⁹ together are arylidenyl. Another specific value for R²⁸ is R²⁸ and R²⁹ together are 4-naphthalenidene-1(4H)-one. Another specific value for R²⁸ is 4-(2-bromo-naphthalen-1-ol).

A specific value for R²⁹ is H. Another specific value for R²⁹ is R²⁸ and R²⁹ together are cycloalkylidenyl. Another specific value for R²⁹ is R²⁸ and R²⁹ together are 2,3,5-trichloro-4-cyclohexylidene-2,5-dienone. Another specific value for R²⁹ is R²⁸ and R²⁹ together are arylidenyl. Another specific value for R²⁹ is R²⁸ and R²⁹ together are 4-naphthalenidene-1(4H)-one. Another specific value for R²⁹ is 4-(2-bromo-naphthalen-1-ol).

For the compounds of formula (VIII):

A specific value for R³⁰ is alkyl. Another specific value for R³⁰ is aryl. Another specific value for R³⁰ is aryl alkyl. Another specific value for R³⁰ is m-NO₂-benzyl. Another specific value for R³⁰ is p-NO₂-benzyl.

For the compounds of formula (IX):

A specific value for X¹¹ is N. Another specific value for X¹¹ is C.

A specific value for X¹² is N. Another specific value for X¹² is C.

A specific value for X¹³ is N. Another specific value for X¹³ is C.

A specific value for X¹⁴ is N. Another specific value for X¹⁴ is C.

A specific value for X¹⁵ is N. Another specific value for X¹⁵ is C.

A specific value for R³¹ is that R³¹ is absent. Another specific value for R³¹ is R³¹ and R³² together are oxo (═O). Another specific value for R³¹ is H. Another specific value for R³¹ is nitro.

A specific value for R³² is that R³¹ is absent. Another specific value for R³² is R³¹ and R³² together are oxo (═O). Another specific value for R³² is H. Another specific value for R³² is nitro.

A specific value for R³³ is that R³³ is absent. Another specific value for R³³ is H. Another specific value for R³³ is heterocycle. Another specific value for R³³ is 2-(4-bromothiophene). Another specific value for R³³ is R³³ and R³⁴ together form a heterocycle. Another specific value for R³³ is R³³ and R³⁴ together form 2-(3,5-dimethylphenyl)isothiazole-3(2H)-thione.

A specific value for R³⁴ is that R³⁴ is absent. Another specific value for R³³ is R³³ and R³⁴ together form a heterocycle. A specific value for R³⁴ is that R³³ and R³⁴ together form 2-(3,5-dimethylphenyl)isothiazole-3(2H)-thione.

A specific value for R³⁵ is H. Another specific value for R³⁵ is that R³⁵ is absent. Another specific value for R³⁵ is alkyl. Another specific value for R³⁵ is 4-(2-ethyl)morpholine. Another specific value for R³⁵ is cyano.

A specific value for R³⁶ is that R³⁶ is absent. Another specific value for R³⁶ is alkyl. Another specific value for R³⁶ is methyl. Another specific value for R³⁶ is methyl 2-acetate. Another specific value for R³⁶ is R³⁶ and R³⁷ together are oxo (═O).

A specific value for R³⁷ is that R³⁷ is absent. Another specific value for R³⁷ is alkyl. Another specific value for R³⁷ is methyl. Another specific value for R³⁷ is methyl 2-acetate. Another specific value for R³⁷ is R³⁶ and R³⁷ together are oxo (═O).

A specific value for R³⁸ is H. Another specific value for R³⁸ is that R³⁸ is absent. Another specific value for R³⁸ is aryl. Another specific value for R³⁸ is phenyl.

A specific value for R³⁹ is H. Another specific value for R³⁹ is SR^{z}, wherein R^{z} is a heterocycle. Another specific value for R³⁹ is 2-(thiobenzo[d]thiazole).

A specific value for R⁴⁰ is that R⁴⁰ is absent. A specific value for R⁴⁰ is H. Another specific value for R⁴⁰ is nitro. Another specific value for R⁴⁰ is halo. Another specific value for R⁴⁰ is bromo. Another specific value for R⁴⁰ is R⁴⁰ and R⁴¹ together are oxo (═O).

A specific value for R⁴¹ is that R⁴¹ is absent. A specific value for R⁴¹ is H. Another specific value for R⁴¹ is nitro. Another specific value for R⁴¹ is halo. Another specific value for R⁴¹ is bromo. Another specific value for R⁴¹ is R⁴⁰ and R⁴¹ together are oxo (═O).

A specific value for R⁴² is H. Another specific value for R⁴² is alkoxy. Another specific value for R⁴² is methoxy.

For the compounds of formula (X):

A specific value for X¹⁶ is O.

A specific value for X¹⁷ is O.

A specific value for R⁴³ is H.

A specific value for R⁴⁴ is H.

A specific value for R⁴⁵ is H.

A specific value for R⁴⁶ is H.

A specific value for R⁴⁷ is H. Another specific value for R⁴⁷ is halo. Another specific value for R⁴⁷ is chloro.

A specific value for R⁴⁸ is H. Another specific value for R⁴⁸ is alkoxy. Another specific value for R⁴⁸ is methoxy.

A specific value for R⁴⁹ is H.

A specific value for R⁵⁰ is H.

For the compounds of formula (XI):

A specific value for X¹⁸ is N.

A specific value for X¹⁹ is N.

A specific value for X²⁰ is N.

A specific value for R⁵¹ is H.

A specific value for R⁵² is aryl. Another specific value for R⁵² is phenyl.

A specific value for R⁵³ is H.

A specific value for R⁵⁴ is hydroxyl.

A specific value for R⁵⁵ is aryl. Another specific value for R⁵⁵ is phenyl.

A specific value for R⁵⁶ is that R⁵⁶ is absent.

A specific value for n is 1.

For the compounds of formula (XII):

A specific value for X²¹ is N.

A specific value for R⁵⁷ is 6-Br.

A specific value for R⁵⁸ is 3-Br.

A specific value for R⁵⁹ is alkyl. Another specific value for R⁵⁹ is aryl alkyl. Another specific value for R⁵⁹ is 1-(3-(2,4-dimethoxyphenylamino)propan-2-ol).

A specific value for n1 is 1.

A specific value for n2 is 1.

For the compounds of formula (XIII):

A specific value for X²² is NH.

A specific value for R⁶⁰ is H.

A specific value for R⁶¹ is C(═O)OR^{t}, wherein R^{t} is alkyl, alkenyl, aryl or cycloxyl. Another specific value for R⁶¹ is methylcarboxylate.

A specific value for R⁶² is aryl. Another specific value for R⁶² is p-ethoxyphenol.

A specific value for R⁶³ is C(═O)OR^{t}, wherein R^{t} is alkyl, alkenyl, aryl or cycloxyl. Another specific value for R⁶³ is methylcarboxylate.

A specific value for R⁶⁴ is H.

TABLE I
Novel Antagonists of the Human Fatty Acid Synthase Thioesterase

| Compound | | |
|---|---|---|
| Identifier and | | |
| No. | Chemical Name (IUPAC) | Chemical Structure |
| RDR019 (1) | 5-((5-(2-bromo-5-methylphenyl)furan-2-yl)methylene)-2- thioxodihydropyrimidine-4,6(1H,5H)-dione | 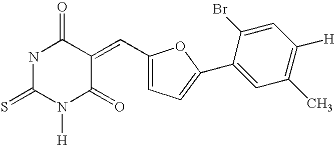 |
| RDR102 (2) | (Z)-5-((5-(2-bromo-4-nitrophenyl)furan-2-yl)methylene)- 1-methylpyrimidine-2,4,6(1H,3H,5H)-trione | 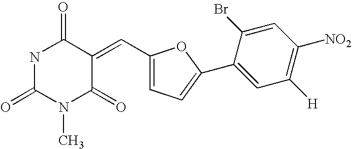 |
| RDR924 (3) | 5-((5-(4-methoxy-2-nitrophenyl)furan-2-yl)methylene)-2- thioxodihydropyrimidine-4,6(1H,5H)-dione | 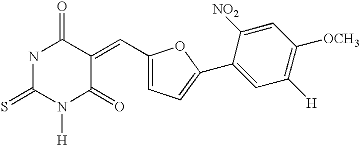 |
| RDR423 (4) | 4-(5-((4,6-dioxo-2-thioxotetrahydropyrimidin-5(6H)- ylidene)methyl)furan-2-yl)benzoic acid | 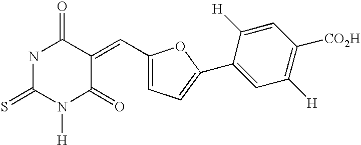 |
| RDR256 (5) | 5-((5-(2-hydroxy-5-nitrophenyl)furan-2- yl)methylene)pyrimidine-2,4,6(1H,3H,5H)-trione | 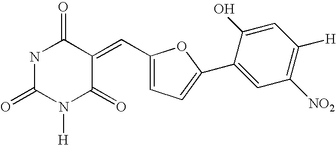 |
| RDR317 (6) | 2-(5-((2,4,6-trioxotetrahydropyrimidin-5(6H)- ylidene)methyl)furan-2-yl)benzoic acid | 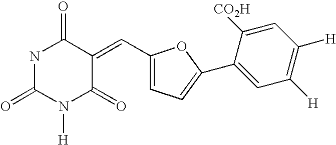 |
| RDR755 (7) | (Z)-1-(2,4-dimethoxyphenyl)-5-((E)-4-phenylbut-3- enylidene)pyrimidine-2,4,6(1H,3H,5H)-trione | 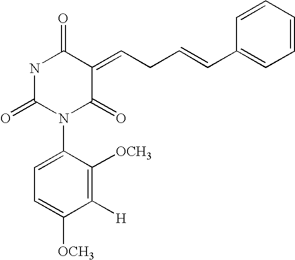 |
| RDR914 (8) | (Z)-5-((E)-4-(2-nitrophenyl)but-3-enylidene)-1- phenylpyrimidine-2,4,6(1H,3H,5H)-trione | 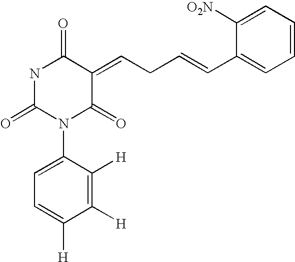 |
| RDR203 (9) | (Z)-1-(3-methoxyphenyl)-5-((E)-4-(2-nitrophenyl)but-3- enylidene)pyrimidine-2,4,6(1H,3H,5H)-trione | 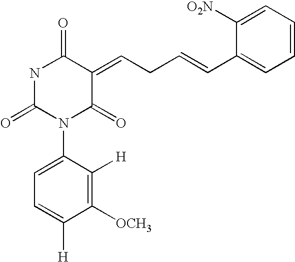 |
| RDR057 (10) | (Z)-5-(2-(4-(dimethylamino)phenyl)ethylidene)-1-(2- fluorophenyl)-2-thioxodihydropyrimidine-4,6(1H,5H)- dione | 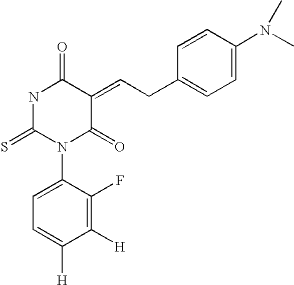 |
| RDR506 (11) | (Z)-1-(4-ethoxyphenyl)-5-(2-(4- ethoxyphenyl)ethylidene)pyrimidine-2,4,6(1H,3H,5H)- trione | 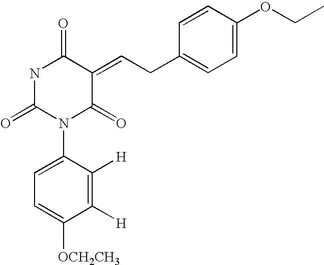 |
| RDR564 (12) | (Z)-1-m-tolyl-5-((o-tolylamino)methylene)pyrimidine- 2,4,6(1H,3H,5H)-trione | 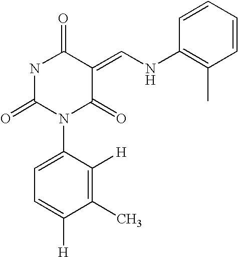 |
| 5839909 (13) | (Z)-4-(4-hydroxybenzylidene)-3-methyl-1-(4- phenylthiazol-2-yl)-1H-pyrazol-5(4H)-one | 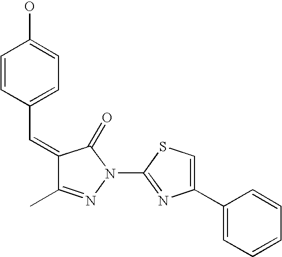 |
| 5587103 (14) | (E)-4-(4-chlorobenzylidene)-1-(3,4- dichlorophenyl)pyrazolidine-3,5-dione | 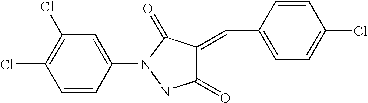 |
| 5786434 (15) | (Z)-1-(3-bromophenyl)-4-(5-chloro-2- methoxybenzylidene)pyrazolidine-3,5-dione | 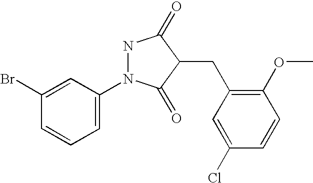 |
| 5865749 (16) | (E)-4-(2,4-dichloro-5-nitrobenzylidene)-3-hydroxy-1- phenyl-1H-pyrazol-5(4H)-one | 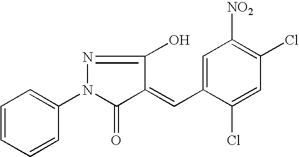 |
| 5215341 (17) | 1,1′-(1,2-phenylene)bis(1H-pyrrole-2,5-dione) | 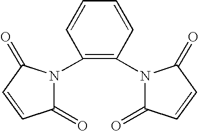 |
| 5992802 (18) | (E)-4-(2-oxoindolin-3-ylidene)-1-phenylpyrazolidine-3,5- dione | 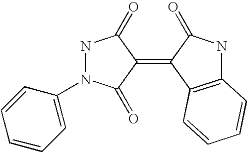 |
| 6237848 (19) | 1-(4-(difluoromethylthio)phenyl)-1H-pyrrole-2,5-dione | 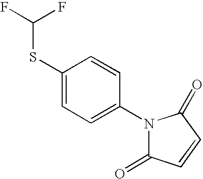 |
| 6238046 (20) | 1-(2-bromo-4-methylphenyl)-1H-pyrrole-2,5-dione | 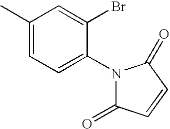 |
| 5621839 (21) | 1-(4-acetylphenyl)-1H-pyrrole-2,5-dione | 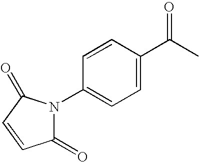 |
| 5627858 (22) | methyl 4-(2,5-dioxo-2,5-dihydro-1H-pyrrol-1-yl)benzoate | 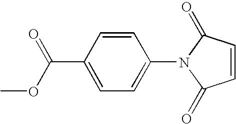 |
| 6237946 (23) | 1-(2-(trifluoromethylthio) phenyl)-1H-pyrrole-2,5-dione | 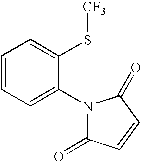 |
| 5842540 (24) | (Z)-5-(5-((2,4-dioxothiazolidin-5-ylidene)methyl)furan-2- yl)-2-hydroxybenzoic acid | 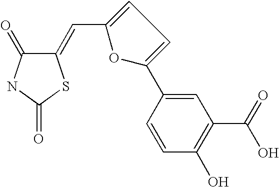 |
| 6222372 (25) | (E)-5-((5-(4-chlorophenylthio)furan-2-yl)methylene)-3- methyl-2-thioxothiazolidin-4-one | 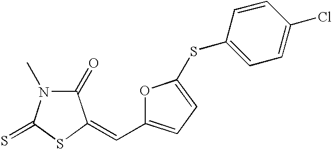 |
| 5550263 (26) | (E)-3-allyl-5-(3-(benzyloxy)benzylidene)-2- thioxothiazolidin-4-one | 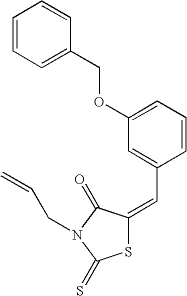 |
| 6200627 (27) | (E)-2-thioxo-3-(4-(2-(m- tolyloxy)ethoxy)benzylideneamino)thiazolidin-4-one | 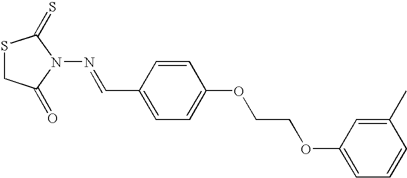 |
| 6238569 (28) | 1-(4-(dimethylamino)phenyl)-1H-pyrrole-2,5-dione | 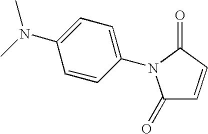 |
| 5761778 (29) | (E)-1-(4-methoxyphenyl)-2,5-dioxopyrrolidin-3-yl N-4- chlorobenzyl-N′-phenylcarbamimidothioate | 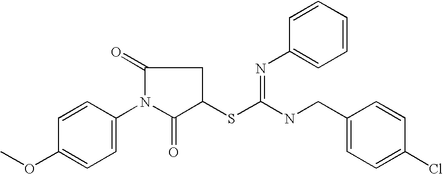 |
| 5605471 (30) | (E)-methyl 4-((3-methyl-4-oxo-2-thioxothiazolidin-5- ylidene)methyl) benzoate | 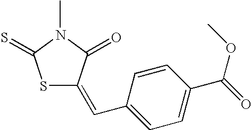 |
| 5399387 (31) | 2-(3-fluorophenyl)-5-(3-nitrophenyl)-1,3,4-oxadiazole | 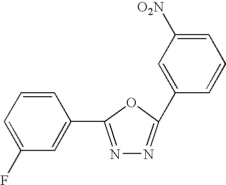 |
| 5158511 (32) | (E)-4-chloro-N-(2,3,5-trichloro-4-oxocyclohexa-2,5- dienylidene)benzenesulfonamide | 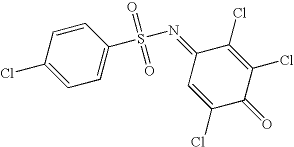 |
| 6165268 (33) | (E)-4-fluoro-N-(4-oxonaphthalen-1(4H)- ylidene)benzenesulfonamide | 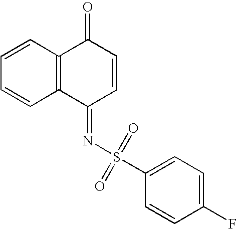 |
| 6155033 (34) | N-(3-bromo-4-hydroxynaphthalen-1-yl)-4- ethylbenzenesulfonamide | 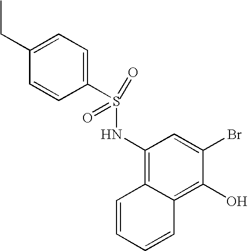 |
| 5155680 (35) | 3-(3-nitrophenyl)-2-thiocyanatopropane nitrile | 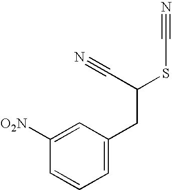 |
| 5155679 (36) | 3-(4-nitrophenyl)-2-thiocyanatopropane nitrile | 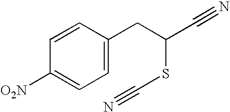 |
| 5670760 (37) | 2-(5,7-dinitroquinolin-8-ylthio)benzo[d]thiazole | 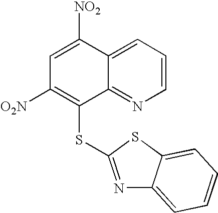 |
| 5809324 (38) | methyl 2-(6-bromo-2-(2-morpholinoethyl)-4- phenylquinazolin-3(4H)-yl)acetate | 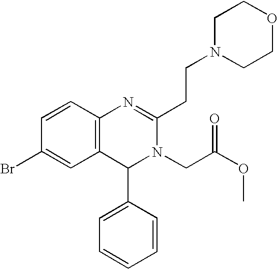 |
| 5760449 (39) | 2-(4-methoxyphenyl)cyclohexa-2,5-diene-1,4-dione | 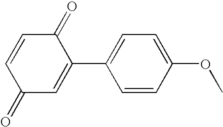 |
| 5763728 (40) | 2-(3-chlorophenyl)cyclohexa-2,5-diene-1,4-dione | 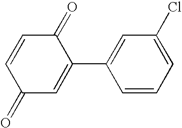 |
| 6108152 (41) | 3-hydroxy-2,4-diphenyl-4,10-dihydroindeno[1,2- b]pyrazolo[4,3-e]yridine-5(2H)-one | 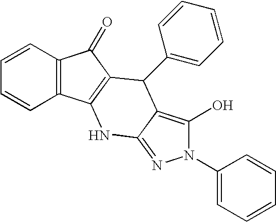 |
| 5869438 (42) | (E)-2-cyano-3-(2,4-dichlorophenyl)-N-((tetrahydrofuran-2- yl)methyl)acrylamide | 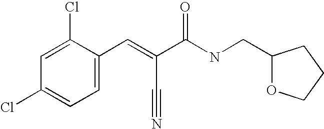 |
| 5653580 (43) | 1-(3,6-dibromo-9H-carbazol-9-yl)-3-(2,4- dimethoxyphenylamino)propan-2-ol | 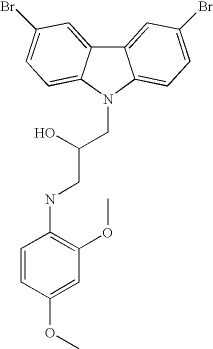 |
| 6368521 (44) | dimethyl 4-(4-ethoxyphenyl)-1,4-dihydropyridine-3,5- dicarboxylate | 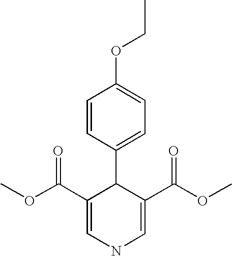 |
| 5630339 (45) | 2-(3,5-dimethylphenyl)-8-methoxy-4,4-dimethyl-4,5- dihydroisothiazolo[5,4-c]quinoline-1(2H)-thione | 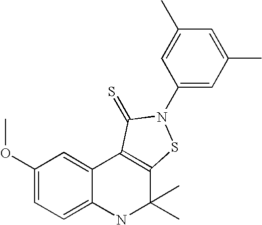 |
| 6238755 (46) | 1-(2,5-dimethoxyphenyl)-5-(3-phenylpropyl)pyrimidine- 2,4,6(1H,3H,5H)-trione | 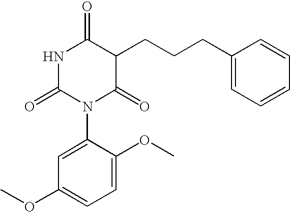 |
| 5843019 (47) | 5-((5-(2-bromo-4-methylphenyl)furan-2-yl)methylene)-2- thioxodihydropyrimidine-4,6(1H,5H)-dione | 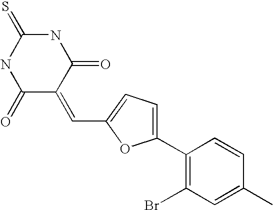 |
| 5988102 (48) | (Z)-5-((5-(2-bromo-4-nitrophenyl)furan-2-yl)methylene)- 1-methylpyrimidine-2,4,6(1H,3H,5H)-trione | 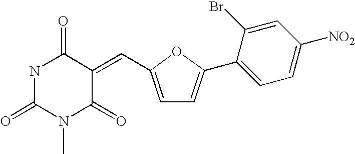 |
| 5809914 (49) | (E)-5-((E)-3-(2-nitrophenyl)allylidene)-1- phenylpyrimidine-2,4,6(1H,3H,5H)-trione | 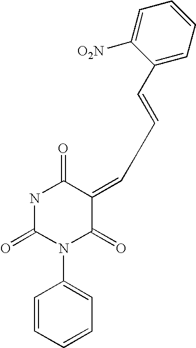 |
| 5182851 (50) | 5-(4-bromothiophen-2-yl)-2,4,7-trioxo-1,2,3,4,7,8- hexahydropyrido[2,3-d]pyrimidine-6-carbonitrile | 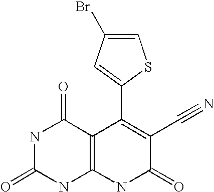 |
| 6238057 (51) | (Z)-5-(4-(dimethylamino)benzylidene)-1-(2-fluorophenyl)- 2-thioxodihydropyrimidine-4,6(1H,5H)-dione | 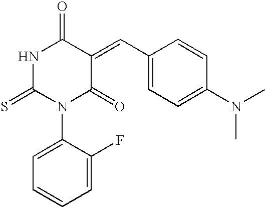 |
| 5377924 (52) | 5-((5-(4-methoxy-2-nitrophenyl)furan-2-yl)methylene)-2- thioxodihydropyrimidine-4,6(1H,5H)-dione | 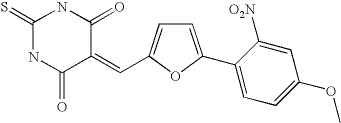 |
| 5376423 (53) | 4-(5-((4,6-dioxo-2-thioxotetrahydropyrimidin-5(6H)- ylidene)methyl)furan-2-yl)benzoic acid | 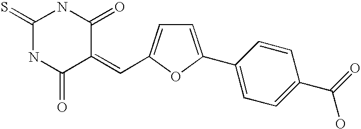 |
| 6238616 (54) | (Z)-5-((E)-3-(furan-2-yl)allylidene)-1-(2- methoxyphenyl)pyrimidine-2,4,6(1H,3H,5H)-trione | 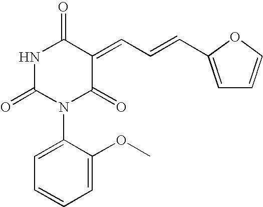 |
| 5810443 (55) | (E)-1-ethyl-5-(furan-3-ylmethylene)-2- thioxodihydropyrimidine-4,6(1H,5H)-dione | 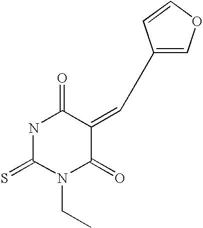 |
| 5810581 (56) | (Z)-1-(4-chlorophenyl)-5-((1-methyl-1H-pyrrol-2- yl)methylene)-2-thioxodihydropyrimidine-4,6(1H,5H)- dione | 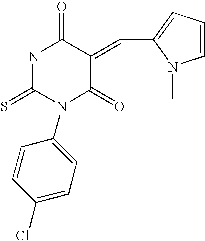 |
| 5810452 (57) | (E)-1-ethyl-5-((1-methyl-1H-pyrrol-2-yl)methylene)-2- thioxodihydropyrimidine-4,6(1H,5H)-dione | 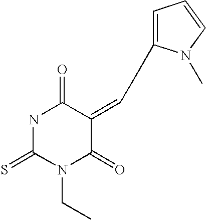 |
| 5810505 (58) | (Z)-5-((1H-pyrrol-2-yl)methylene)-1-methyl-3-phenyl-2- thioxodihydropyrimidine-4,6(1H,5H)-dione | 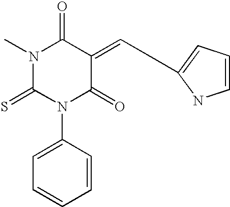 |

TABLE II
Novel Antagonists of the Human Fatty Acid Synthase Thioesterase

| Compound | | | |
|---|---|---|---|
| Identifier and | | | |
| No. | Chemical Structure | Compound of Formula: | Substituent Values |
| RDR019 (1) | 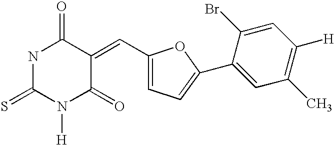 | 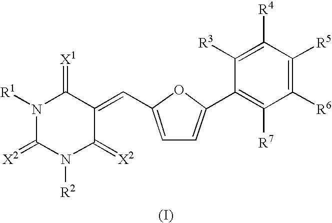 | X¹ = O; X² = S; X³ = O; R¹ = H; R² = H; R³ = Br; R⁴ = H; R⁵ = H; R⁶ = CH₃; and R⁷ = H. |
| RDR102 (2) | 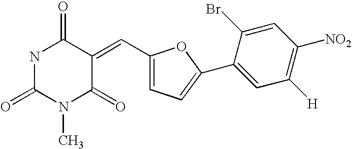 | 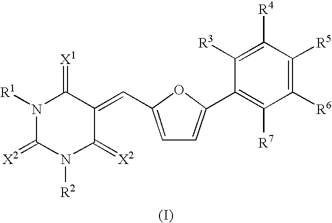 | X¹ = O; X² = O; X³ = O; R¹ = H; R² = CH₃; R³ = Br; R⁴ = H; R⁵ = NO₂; R⁶ = H; and R⁷ = H. |
| RDR924 (3) | 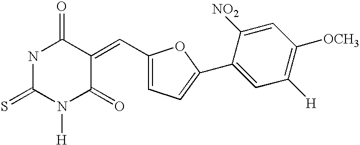 | 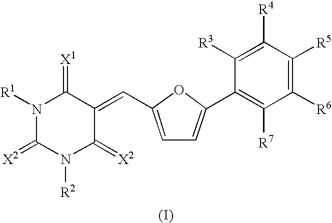 | X¹ = O; X² = S; X³ = O; R¹ = H; R² = H; R³ = NO₂; R⁴ = H; R⁵ = OCH₃; R⁶ = H; and R⁷ = H. |
| RDR423 (4) | 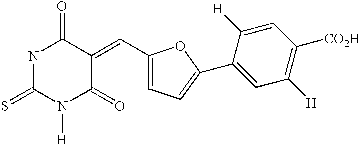 | 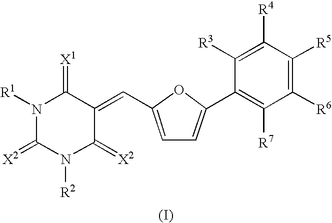 | X¹ = O; X² = S; X³ = O; R¹ = H; R² = H; R³ = H; R⁴ = H; R⁵ = CO₂H; R⁶ = H; and R⁷ = H. |
| RDR256 (5) | 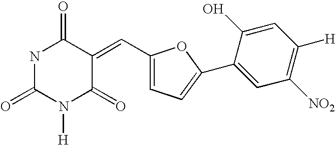 | 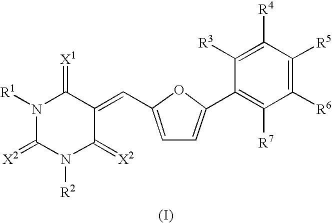 | X¹ = O; X² = O; X³ = O; R¹ = H; R² = H; R³ = OH; R⁴ = H; R⁵ = H; R⁶ = NO₂; and R⁷ = H. |
| RDR317 (6) | 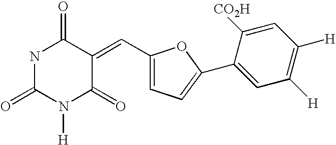 | 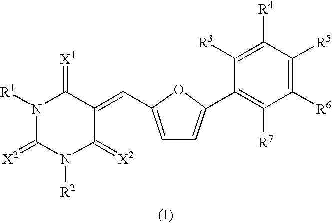 | X¹ = O; X² = O; X³ = O; R¹ = H; R² = H; R³ = CO₂H; R⁴ = H; R⁵ = H; R⁶ = H; and R⁷ = H. |
| RDR755 (7) | 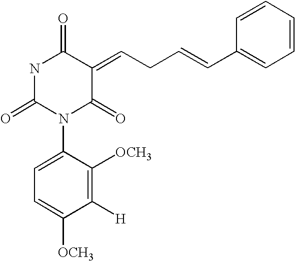 | 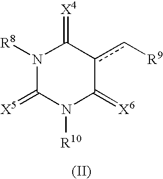 | Optional double bond is present; X⁴ = O; X⁵ = O; X⁶ = O; R⁸ = H; R⁹ = CH₂CH═CH—Ph; and R¹⁰ = 1,3-di-OCH₃—Ph. |
| RDR914 (8) | 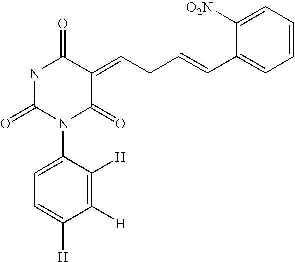 | 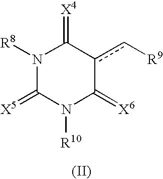 | Optional double bond is present; X⁴ = O; X⁵ = O; X⁶ = O; R⁸ = H; R⁹ = CH₂CH═CH-(o-NO₂)Ph; and R¹⁰ = Ph. |
| RDR203 (9) | 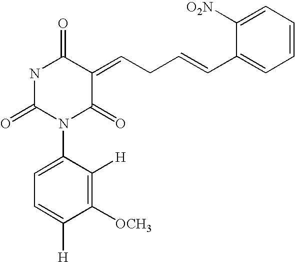 | 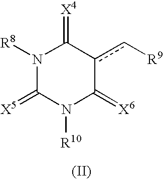 | Optional double bond is present; X⁴ = O; X⁵ = O; X⁶ = O; R⁸ = H; R⁹ = CH₂CH═CH-(o-NO₂)Ph; and R¹⁰ = (m-OCH₃)-Ph. |
| RDR057 (10) | 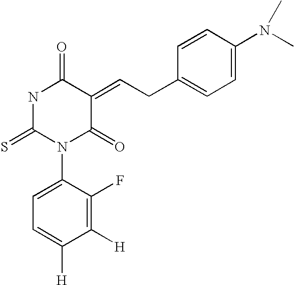 | 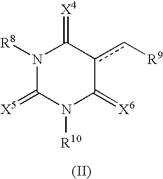 | Optional double bond is present; X⁴ = O; X⁵ = S; X⁶ = O; R⁸ = H; R⁹ = CH₂-(p-N(CH₃)₂)Ph; and R¹⁰ = o-fluorophenyl. |
| RDR506 (11) | 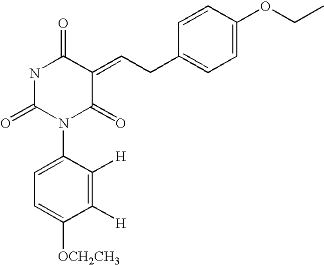 | 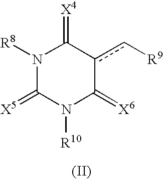 | Optional double bond is present; X⁴ = O; X⁵ = O; X⁶ = O; R⁸ = H; R⁹ = CH₂-(p-OCH₂CH₃)Ph; and R¹⁰ = (p-OCH₂CH₃)-Ph. |
| RDR564 (12) | 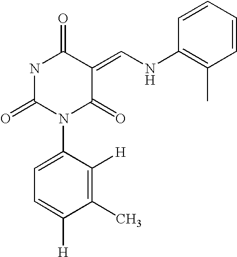 | 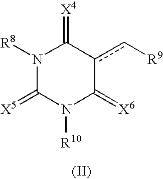 | Optional double bond is present; X⁴ = O; X⁵ = O; X⁶ = O; R⁸ = H; R⁹ = NH-(o-CH₃)Ph; and R¹⁰ = (m-CH₃)Ph. |
| 5839909 (13) | 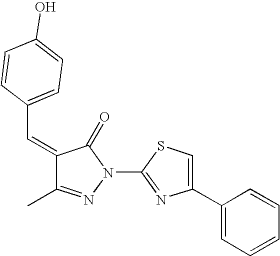 | 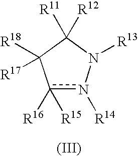 | R¹¹ and R¹² together are oxo (═O); R¹³ = 1-(4-phenylthiazol); R¹⁴ = absent; R¹⁵ = absent; R¹⁶ = CH₃; R¹⁷ and R¹⁸ together are ═CH-p-phenol; and Optional double bond is present. |
| 5587103 (14) | 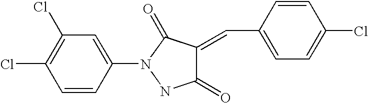 | 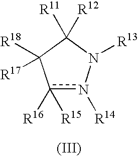 | R¹¹ and R¹² together are oxo (═O); R¹³ = 3,4-dichlorophenyl; R¹⁴ = H; R¹⁵ and R¹⁶ together are oxo (═O); R¹⁷ and R¹⁸ together are ═CH-p-Cl—Ph; and Optional double bond is present. |
| 5786434 (15) | 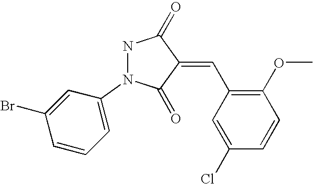 | 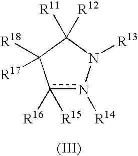 | R¹¹ and R¹² together are oxo (═O); R¹³ = H; R¹⁴ = m-Br—Ph; R¹⁵ and R¹⁶ together are oxo (═O); R¹⁷ and R¹⁸ together are ═CH-(2-OCH₃-5-Cl)-Ph; and Optional double bond is absent. |
| 5865749 (16) | 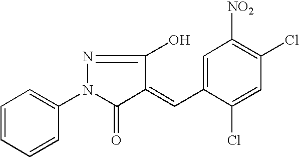 | 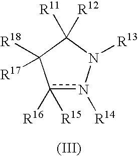 | R¹¹ and R¹² together are oxo (═O); R¹³ = Ph; R¹⁴ = absent; R¹⁵ = absent; R¹⁶ = OH; R¹⁷ and R¹⁸ together are 2,4- dichloro-5-nitrobenzylidene; and Optional double bond is present. |
| 5215341 (17) | 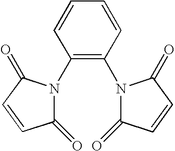 | 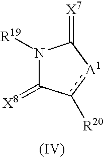 | X⁷ = O; X⁸ = O; A¹ = CH; R¹⁹ = 2-(1H-pyrrole-2,5-dione) phenyl; R²⁰ = H; and Optional bond is present. |
| 5992802 (18) | 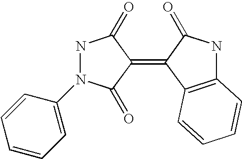 | 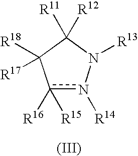 | R¹¹ and R¹² together are oxo (═O); R¹³ = H; R¹⁴ = Ph; R¹⁵ and R¹⁶ together are oxo (═O); R¹⁸ and R¹⁸ together are 4-(1- phenylpyrazolidine- 3,5-dione); and Optional double bond is absent. |
| 6237848 (19) | 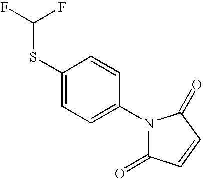 | 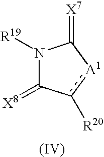 | X⁷ = O; X⁸ = O; A¹ = CH; R¹⁹ = 1-(4-(difluoromethylthio) phenyl); R²⁰ = H; and Optional bond is present. |
| 6238046 (20) | 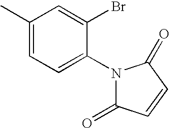 | 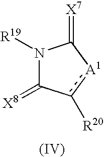 | X⁷ = O; X⁸ = O; A¹ = CH; R¹⁹ = 1-(2-bromo-4- methylphenyl); R²⁰ = H; and Optional bond is present. |
| 5621839 (21) | 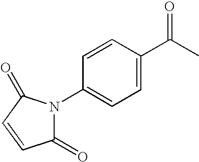 | 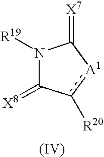 | X⁷ = O; X⁸ = O; A¹ = CH; R¹⁹ = 1-(4-phenylethanone); R²⁰ = H; and Optional bond is present. |
| 5627858 (22) | 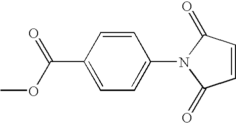 | 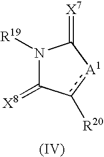 | X⁷ = O; X⁸ = O; A¹ = CH; R¹⁹ = 4-methylbenzoate; R²⁰ = H; and Optional bond is present. |
| 6237946 (23) | 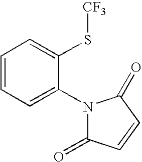 | 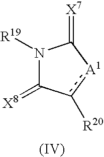 | X⁷ = O; X⁸ = O; A¹ = CH; R¹⁹ = 1-(2- trifluoromethylthio)phenyl); R²⁰ = H; and Optional bond is present. |
| 5842540 (24) | 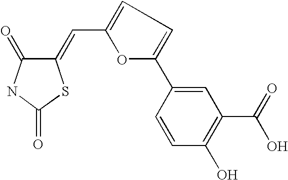 | 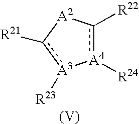 | A² = O; A³ = C; A⁴ = C; R²¹ = 1-(4-hydroxy-3- benzoic acid); R²² = (Z)-5-(methylene) thiazolidine-2,4-dione; R²³ = H R²⁴ = H; and Optional bonds are present. |
| 6222372 (25) | 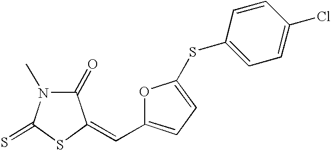 | 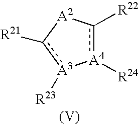 | A² = O; A³ = C; A⁴ = C; R²¹ = (E)-5-(methylene)-3-methyl- 2-thioxothiazolidin-4-one; R²² = (4-chlorophenyl)sulfane; R²³ = H R²⁴ = H; and Optional bonds are present. |
| 5550263 (26) | 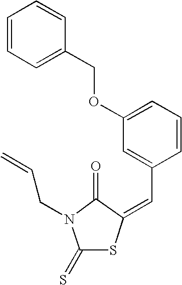 | 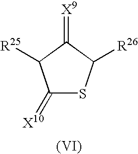 | X⁹ = O; X¹⁰ = S; R²⁵ = CH₂CH═CH₂; and R²⁶ = 1-(3-benzyloxy)- vinylbenzyl. |
| 6200627 (27) | 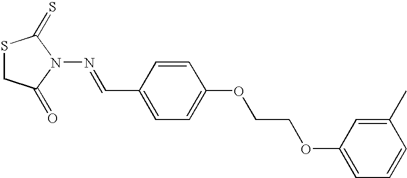 | 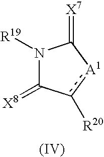 | X⁷ = S; X⁸ = O; A¹ = S; R¹⁹ = (E)-1-(2-(4- ((imino)methyl)phenoxy)ethoxy)- 3-methylbenzene; R²⁰ = H; and Optional bond is absent. |
| 6238569 (28) | 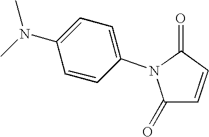 | 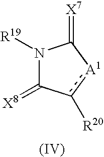 | X⁷ = O; X⁸ = O; A¹ = CH; R¹⁹ = 1-(4-(N,N- dimethylbenzeneamine)); R²⁰ = H; and Optional bond is present. |
| 5761778 (29) | 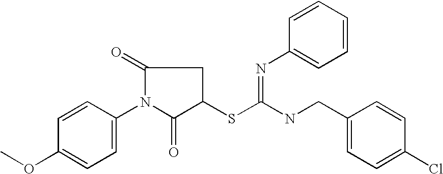 | 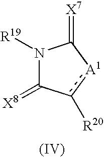 | X⁷ = O; X⁸ = O; A¹ = CH; R¹⁹ = 1-(4-methoxyphenyl); R²⁰ = (E)-N-4-chlorobenzyl-N′- phenylcarbamimidothioate; and Optional bond is absent. |
| 5605471 (30) | 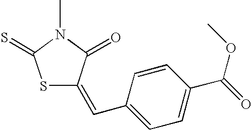 | 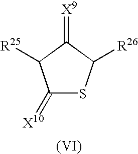 | X⁹ = O; X¹⁰ = S; R²⁵ = CH₃; and R²⁶ = 1-(4-vinylbenzoate). |
| 5399387 (31) | 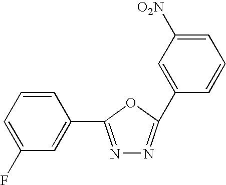 | 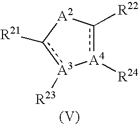 | A² = O; A³ = N; A⁴ = N; R²¹ = 1-(3-F—Ph); R²² = 1-(3-NO₂—Ph); R²³ = absent; R²⁴ = absent; and Optional bonds are present. |
| 5158511 (32) | 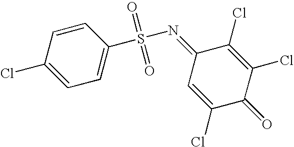 | 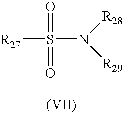 | R²⁷ = p-Cl—Ph; and R²⁸ and R²⁹ together is 2,3,5- trichloro-4-cyclohexylidene-2,5- dienone. |
| 6165268 (33) | 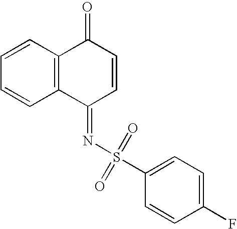 | 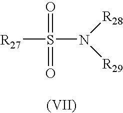 | R²⁷ = p-F—Ph; and R²⁸ and R²⁹ together is 4- naphthalenidene-1(4H)-one. |
| 6155033 (34) | 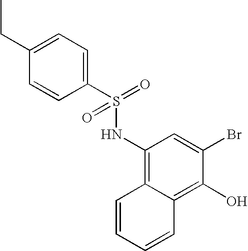 | 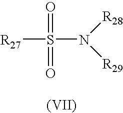 | R²⁷ = p-Et—Ph; and R²⁸ = H; and R²⁹ = 4-(2-bromo-naphthalen-1-ol). |
| 5155680 (35) | 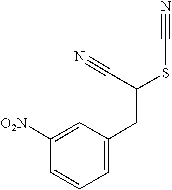 | 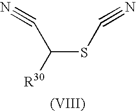 | R³⁰ = m-NO₂-Benzyl |
| 5155679 (36) | 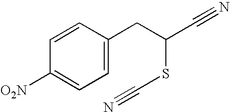 | 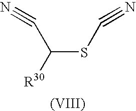 | R³⁰ = p-NO₂-Benzyl |
| 5670760 (37) | 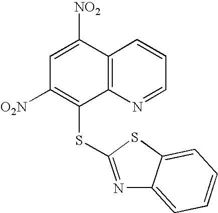 | 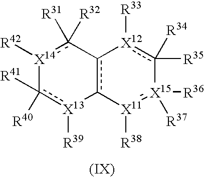 | X¹¹ = N; X¹² = C; X¹³ = C; X¹⁴ = C; X¹⁵ = C; R³¹ = absent; R³² = NO₂; R³³ = H; R³⁴ = absent; R³⁵ = H; R³⁶ = absent; R³⁷ = H; R³⁸ = absent; R³⁹ = 2-(thiobenzo[d]thiazole); |
| # R⁴⁰ = absent; R⁴¹ = NO₂; R⁴² = H; Optional bond at X¹² is present; Optional bond between X¹¹ and X¹⁵ is present; Optional bond at bridgehead is present; Optional bond at X¹³ is present; and Optional bond at X¹⁴ is present. | | | |
| 5809324 (38) | 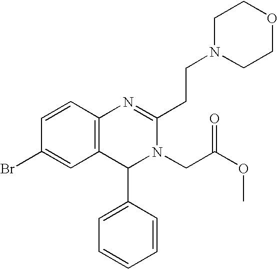 | 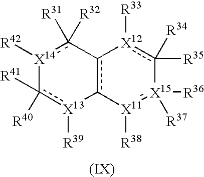 | X¹¹ = C; X¹² = N; X¹³ = C; X¹⁴ = C; X¹⁵ = N; R³¹ = absent; R³² = H; R³³ = absent; R³⁴ = absent; R³⁵ = 4-(2-ethyl)morpholine; R³⁶ = absent; R³⁷ = methyl 2-acetate; R³⁸ = Ph; R³⁹ = H; |
| # R⁴⁰ = absent; R⁴¹ = Br; R⁴² = H; Optional bond at X¹² is present; Optional bond between X¹¹ and X¹⁵ is absent; Optional bond at bridgehead is present; Optional bond at X¹³ is present; and Optional bond at X¹⁴ is present. | | | |
| 5760449 (39) | 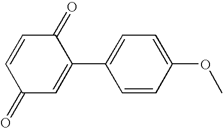 | 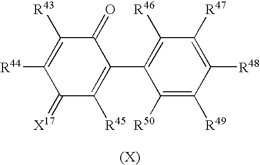 | X¹⁶ = O; X¹⁷ = O; R⁴³ = H; R⁴⁴ = H; R⁴⁵ = H; R⁴⁶ = H; R⁴⁷ = H; R⁴⁸ = OMe; R⁴⁹ = H; and R⁵⁰ = H. |
| 5763728 (40) | 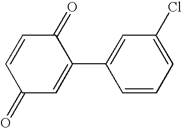 | 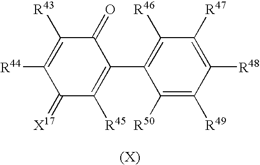 | X¹⁶ = O; X¹⁷ = O; R⁴³ = H; R⁴⁴ = H; R⁴⁵ = H; R⁴⁶ = H; R⁴⁷ = Cl; R⁴⁸ = H; R⁴⁹ = H; and R⁵⁰ = H. |
| 6108152 (41) | 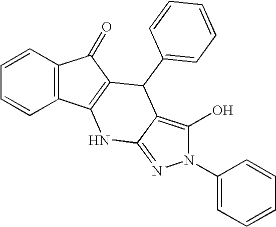 | 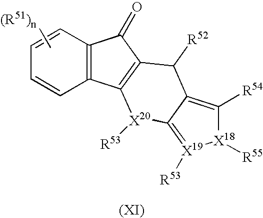 | X¹⁸ = N; X¹⁹ = N; X²⁰ = N; R⁵¹ = H; R⁵² = Ph; R⁵³ = H; R⁵⁴ = OH; R⁵⁵ = Ph; R⁵⁶ = absent; and n = 1. |
| 5869438 (42) | 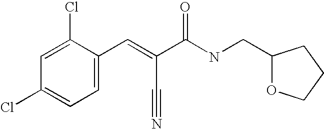 | 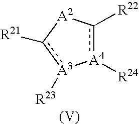 | A² = O; A³ = CH; A⁴ = CH; R²¹ = H; R²² =(E)-2-cyano-3-(2,4- dichlorophenyl)-N-(methyl) acrylamide; R²³ = H; R²⁴ = H; and Optional bonds are absent. |
| 5653580 (43) | 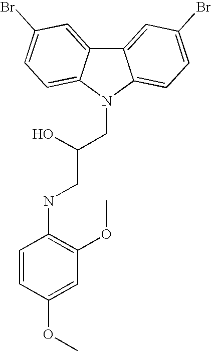 | 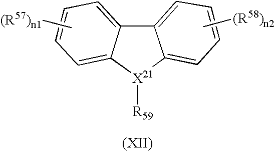 | X²¹ = N; R⁵⁷ = 6-Br; R⁵⁸ = 3-Br; R⁵⁹ = 1-(3-(2,4- dimethoxyphenylamino)propan- 2-ol); n1 = 1; and n2 = 1. |
| 6368521 (44) | 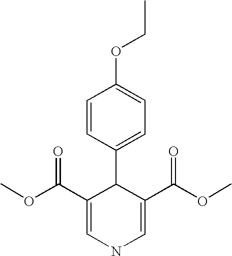 | 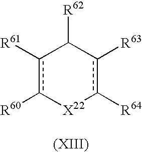 | X²² = NH; R⁶⁰ = H; R⁶¹ = methylformate; R⁶² = p-ethoxyphenyl; R⁶³ = methylformate; R⁶⁴ = H; and Optional bonds are present. |
| 5630339 (45) | 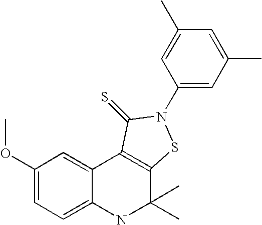 | 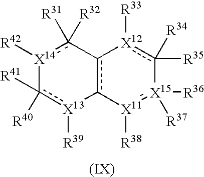 | X¹¹ = N; X¹² = C; X¹³ = C; X¹⁴ = C; X¹⁵ = C; R³¹ = absent; R³² = H; R³³ and R³⁴ together form 2-(3,5- dimethylphenyl)isothiazole-3(2H)- thione; R³⁵ = absent; R³⁶ = Me; R³⁷ = Me; R³⁸ = absent; |
| # R³⁹ = H; R⁴⁰ = absent; R⁴¹ = H; R⁴² = OMe; Optional bond at X¹² ispresent; Optional bond between X¹¹ and X¹⁵ is absent; Optional bond at bridgehead is present; Optional bond at X¹³ is present; and Optional bond at X¹⁴ is present. | | | |
| 6238755 (46) | 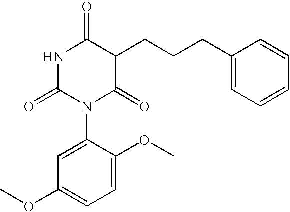 | 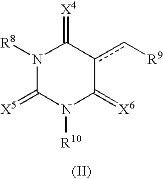 | Optional double bond is absent; X⁴ = O; X⁵ = O; X⁶ = O; R⁸ = H; R⁹ = CH₂CH₂Ph; and R¹⁰ = 2,5-di-OCH₃(Ph). |
| 5843019 (47) | 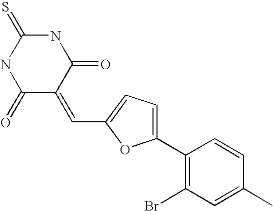 | 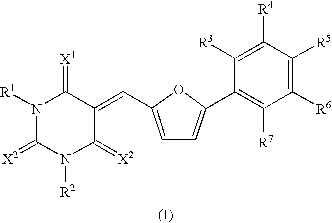 | X¹ = O; X² = S; X³ = O; R¹ = H; R² = H; R³ = Br; R⁴ = H; R⁵ = CH₃; R⁶ = H; and R⁷ = H. |
| 5988102 (48) | 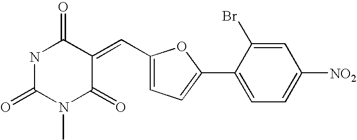 | 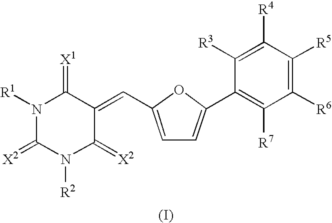 | X¹ = O; X² = O; X³ = O; R¹ = H; R² = CH₃; R³ = Br; R⁴ = H; R⁵ = NO₂; R⁶ = H; and R⁷ = H. |
| 5809914 (49) | 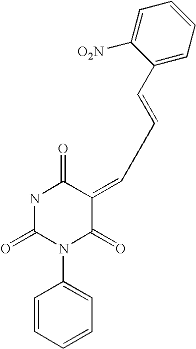 | 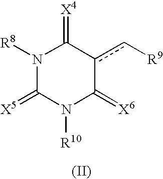 | Optional double bond is present; X⁴ = O; X⁵ = O; X⁶ = O; R⁸ = H; R⁹ = CH═CH(o-NO₂)Ph; and R¹⁰ = Ph. |
| 5182851 (50) | 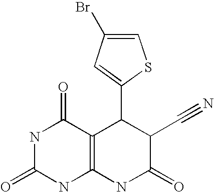 | 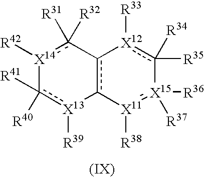 | X¹¹ = N; X¹² = C; X¹³ = N; X¹⁴ = N; X¹⁵ = C; R³¹ and R³² together are oxo (═O); R³³ = 2-(4-bromothiophene); R³⁴ = absent; R³⁵ = cyano; R³⁶ and R³⁷ together are oxo (═O); R³⁸ = H; R³⁹ = H; |
| # R⁴⁰ and R⁴¹ together are oxo (═O); R⁴² = H; Optional bond between X¹¹ and X¹⁵ is absent; Optional bond at bridgehead is present; Optional bond at X¹³ is absent; and Optional bond at X¹⁴ is absent. | | | |
| 6238057 (51) | 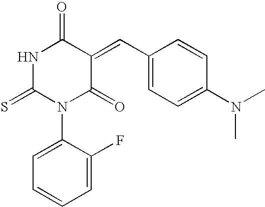 | 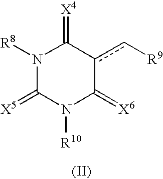 | Optional double bond is present; X⁴ = O; X⁵ = S; X⁶ = O; R⁸ = H; R⁹ = 4-(N,N- dimethylbenzeneamine); R¹⁰ = 1-fluorobenzene. |
| 5377924 (52) | 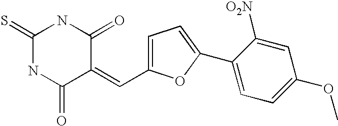 | 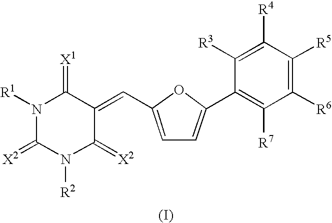 | X¹ = O; X² = S; X³ = O; R¹ = H; R² = H; R³ = NO₂; R⁴ = H; R⁵ = OCH₃; R⁶ = H; and R⁷ = H. |
| 5376423 (53) | 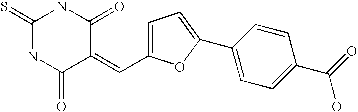 | 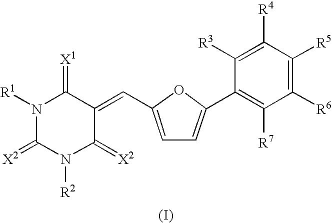 | X¹ = O; X² = S; X³ = O; R¹ = H; R² = H; R³ = H; R⁴ = H; R⁵ = C(═O)OH; R⁶ = H; and R⁷ = H. |
| 6238616 (54) | 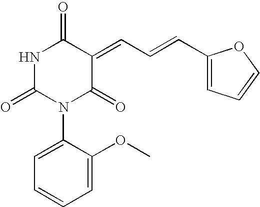 | 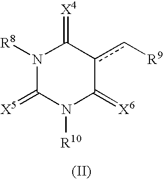 | Optional double bond is present; X⁴ = O; X⁵ = O; X⁶ = O; R⁸ = H; R⁹ = 2-vinylfuran; and R¹⁰ = (o-OCH₃)Ph. |
| 5810443 (55) | 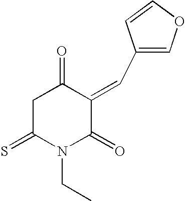 | 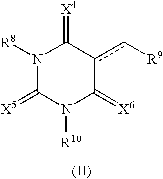 | Optional double bond is present; X⁴ = O; X⁵ = S; X⁶ = O; R⁸ = H; R⁹ = 3-furanyl; and R¹⁰ = CH₂CH₃. |
| 5810581 (56) | 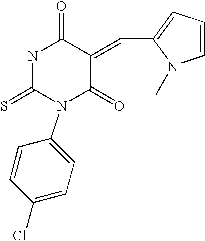 | 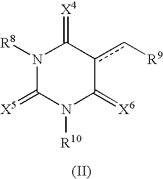 | Optional double bond is present; X⁴ = O; X⁵ = S; X⁶ = O; R⁸ = H; R⁹ = 2-(1-methyl-1H-pyrrole); and R¹⁰ = (p-Cl)Ph. |
| 5810452 (57) | 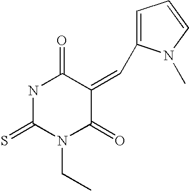 | 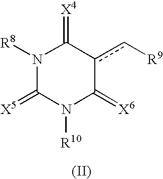 | Optional double bond is present; X⁴ = O; X⁵ = S; X⁶ = O; R⁸ = H; R⁹ = 2-(1-methyl-1H-pyrrole); and R¹⁰ = CH₂CH₃. |
| 5810505 (58) | 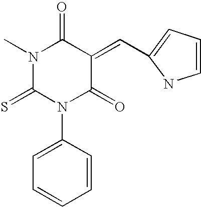 | 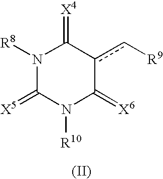 | Optional double bond is present; X⁴ = O; X⁵ = S; X⁶ = O; R⁸ = CH₃; R⁹ = 2-(1H-pyrrole); and R¹⁰ = Ph. |

As used herein, “μg” denotes microgram, “mg” denotes milligram, “g” denotes gram, “μL” denotes microliter, “mL” denotes milliliter, “L” denotes liter, “nM” denotes nanomolar, “μM” denotes micromolar, “mM” denotes millimolar, “M” denotes molar and “nm” denotes nanometer. “Sigma” stands for the Sigma-Aldrich Corp. of St. Louis Mo.

The compounds of the present invention (compounds of Formula I-XIII) are useful in medical therapy or diagnosis. Specifically, the compounds of the present invention are useful in inhibiting FAS. More specifically, the compounds of the present invention are useful in inhibiting the TE domain of the FAS. This can occur in vitro or in vivo. As such, the compounds of the present invention are useful in treating cancer in mammals (e.g., humans), as well inhibiting tumor cell growth in such mammals. The tumor can be a solid tumor and can be located, e.g., in the ovary, breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary gland, or the heart. Additionally, the compounds of the present invention can be administered locally or systemically, alone or in combination with one or more anti-cancer agents.

Anti-Cancer Agents

The compounds of the present invention can optionally be administered with an anti-cancer agent. Anti-cancer or anti-cell proliferation agents include, e.g., nucleotide and nucleoside analogs, such as 2-chloro-deoxyadenosine, adjunct antineoplastic agents, alkylating agents, nitrogen mustards, nitrosoureas, antibiotics, antimetabolites, hormonal agonists/antagonists, androgens, antiandrogens, antiestrogens, estrogen & nitrogen mustard combinations, gonadotropin releasing hotmone (GNRH) analogues, progestrins, immunomodulators, miscellaneous antineoplastics, photosensitizing agents, and skin & mucous membrane agents. See, Physician's Desk Reference (2001).

Suitable adjunct antineoplastic agents include Anzemet® (Hoeschst Marion Roussel), Aredia® (Novartis), Didronel® (MGI), Diflucan® (Pfizer), Epogen® (Amgen), Ergamisol® (Janssen), Ethyol® (Alza), Kytril® (SmithKline Beecham), Leucovorin® (Immunex), Leucovorin® (Glaxo Wellcome), Leucovorin® (Astra), Leukine® (Immunex), Marinol® (Roxane), Mesnex® (Bristol-Myers Squibb Oncology/Immunology, Neupogen (Amgen), Procrit® (Ortho Biotech), Salagen® (MGI), Sandostatin® (Novartis), Zinecard® (Pharmacia & Upjohn), Zofran® (Glaxo Wellcome) and Zyloprim® (Glaxo Wellcome).

Suitable miscellaneous alkylating agents include Myleran® (Glaxo Wellcome), Paraplatin® (Bristol-Myers Squibb Oncology/Immunology), Platinol® (Bristol-Myers Squibb Oncology/Immunology) and Thioplex® (Immunex).

Suitable nitrogen mustards include Alkeran® (Glaxo Wellcome), Cytoxan® (Bristol-Myers Squibb Oncology/Immunology), Ifex® (Bristol-Myers Squibb Oncology/Immunology), Leukeran® (Glaxo Wellcome) and Mustargen® (Merck).

Suitable nitrosoureas include BiCNU® (Bristol-Myers Squibb Oncology/Immunology), CeeNU® (Bristol-Myers Squibb Oncology/Immunology), Gliadel® (Rhône-Poulenc Rover) and Zanosar® (Pharmacia & Upjohn).

Suitable antibiotics include Adriamycin PFS/RDF® (Pharmacia & Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia & Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology).

Suitable antimetabolites include Cytostar-U® (Pharmacia & Upjohn), Fludara® (Berlex), Sterile FUDR® (Roche Laboratories), Leustatin® (Ortho Biotech), Methotrexate® (Immunex), Parinethol® (Glaxo Wellcome), Thioguanine® (Glaxo Wellcome) and Xeloda® (Roche Laboratories).

Suitable androgens include Nilandron® (Hoechst Marion Roussel) and Teslac® (Bristol-Myers Squibb Oncology/Immunology).

Suitable antiandrogens include Casodex® (Zeneca) and Eulexin® (Schering).

Suitable antiestrogens include Arimidex® (Zeneca), Fareston® (Schering), Femara® (Novartis) and Nolvadex® (Zeneca).

Suitable estrogen & nitrogen mustard combinations include Emcyt® (Pharmacia & Upjohn).

Suitable estrogens include Estrace® (Bristol-Myers Squibb) and Estrab® (Solvay).

Suitable gonadotropin releasing hormone (GNRH) analogues include Leupron Depot® (TAP) and Zoladex® (Zeneca).

Suitable progestins include Depo-Provera® (Pharmacia & Upjohn) and Megace® (Bristol-Myers Squibb Oncology/Immunology).

Suitable immunomodulators include Erganisol® (Janssen) and Proleukin® (Chiron Corporation).

Suitable miscellaneous antineoplastics include Camptosar® (Pharmacia & Upjohn), Celestone® (Schering), DTIC-Dome® (Bayer), Elspar® (Merck), Etopophos® (Bristol-Myers Squibb Oncology/Immunology), Etopoxide® (Astra), Gemzar® (Lilly), Hexalen® (U.S. Bioscience), Hycantin® (SmithKline Beecham), Hydrea® (Bristol-Myers Squibb Oncology/Immunology), Hydroxyurea® (Roxane), Intron A® (Schering), Lysodren® (Bristol-Myers Squibb Oncology/Immunology), Navelbine® (Glaxo Wellcome), Oncaspar® (Rhône-Poulenc Rover), Oncovin® (Lilly), Proleukin® (Chiron Corporation), Rituxan® (IDEC), Rituxan® (Genentech), Roferon-A® (Roche Laboratories), Taxol® (Bristol-Myers Squibb Oncology/Immunology), Taxotere® (Rhône-Poulenc Rover), TheraCys® (Pasteur Merieux Connaught), Tice BCG® (Organon), Velban® (Lilly), VePesid® (Bristol-Myers Squibb Oncology/Immunology), Vesanoid® (Roche Laboratories) and Vumon® (Bristol-Myers Squibb Oncology/Immunology).

Suitable photosensitizing agents include Photofrin® (Sanofi).

Specifically, the anti-cancer or anti-cell proliferation agent can include Taxol® (paclitaxol), a niticoxide like compound, or NicOx (NCX-4016).

Taxol® (paclitaxol) is chemically designated as 5β,20-Epoxy-1,2α,4,7β,10β,13α-hexahydroxytax-11-en-9-one 4,10-diacetate 2-benzoate 13-ester with (2R,3S)—N-benzoyl-3-phenylisoserine.

A niticoxide like compound includes any compound (e.g., polymer) to which is bound a nitric oxide releasing functional group. Suitable niticoxide like compounds are disclosed, e.g., in U.S. Pat. No. 5,650,447 and S-nitrosothiol derivative (adduct) of bovine or human serum albumin. See, e.g., Marks et al. (1995).

NCX-4016 is chemically designated as 2-acetoxy-benzoate 2-(nitroxymethyl)-phenyl ester, and is an antithrombitic agent.

It is appreciated that those skilled in the art understand that the drug useful in the present invention is the biologically active substance present in any of the drugs or agents disclosed above. For example, Taxol® (paclitaxol) is typically available as an injectable, slightly yellow viscous solution. The drug, however, is a crystalline powder with the chemical name 5β,20-Epoxy-1,2α,4,7β,10β,13α-hexahydroxytax-11-en-9-one 4,10-diacetate 2-benzoate 13-ester with (2R,3S)—N-benzoyl-3-phenylisoserine. Physician's Desk Reference, 53rd Ed., pp. 1059-1067.

Pharmaceutical Formulations

The compounds of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise one or more compounds of the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of a given condition.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

Effective dose of active ingredient depends at least on the nature of the condition being treated, toxicity, whether the compound is being used prophylactically (lower doses), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

Routes of Administration

One or more compounds of the invention (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally.

Combination Therapy

Active ingredients of the invention are also used in combination with other active ingredients. Such combinations are selected based on the condition to be treated, cross-reactivities of ingredients and pharmaco-properties of the combination.

It is also possible to combine any compound of the invention with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide “synergy” and “synergistic effect”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together.

Pharmaceutical kits useful in the present invention, which include a therapeutically effective amount of a pharmaceutical composition that includes a compound of component (a) and one or more compounds of component (b), in one or more sterile containers, are also within the ambit of the present invention. Sterilization of the container may be carried out using conventional sterilization methodology well known to those skilled in the art. Component (a) and component (b) may be in the same sterile container or in separate sterile containers. The sterile containers or materials may include separate containers, or one or more multi-part containers, as desired. Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

The present invention can be illustrated by the following non-limiting examples.

### EXAMPLE I

Material and Methods

Expression and Purification of the FAS TE. Expression of the recombinant thioesterase domain of FAS using pTrcHis-TOPO vector (Invitrogen) was as described in Kridel et al. (2004). Large-scale expression and purification was performed by Invitrogen Corporation (Madison, Wis.).

Compound Screening. A primary screen of 36,500 compounds from the DIVERSet Collection (Chembridge) was performed in 96-well Fluorotrac 200 plates (Greiner) using 4-methylumbelliferyl heptanoate (4-MUH, Sigma) as a fluorogenic substrate (Jacks et al., 1967; Guilbault et al., 1969). The optimal substrate concentration was 120 μM 4-MUH, or approximately 3×Kₘ. Briefly, reaction mixtures contained FAS TE in Buffer A (45 μl; 100 mM Tris-HCl, 50 mM NaCl, pH 7.5) or Buffer A alone. Controls included protein solution plus vehicle (DMSO) to determine untreated enzyme activity and Buffer A plus DMSO to quantify background hydrolysis of the fluorogenic substrate. Library compounds (5 μL) or a 10% (v/v) DMSO solution (control) were added to yield final concentrations of approximately 12.5 μM, and the background fluorescence was measured at 360/435 nm. The plates were incubated at 37° C. for 30 minutes before adding 4-MUH in 5 μL DMSO:Buffer A (1:1). Plates were incubated at 37° C. for 60 minutes and assayed at 360/435 nm. Compounds that inhibited enzymatic activity ≧40% were further studied.

Secondary Fluorogenic Screen. Lead compounds were purchased from Chembridge (www.hit2lead.com). Each compound was tested at concentrations of 1 to 100 μM. Data points were collected in triplicate. Reaction volumes contained 2.5 μL of each dilution or vehicle (DMSO) with 45 μL of 500 nM FAS TE in Buffer A or Buffer A alone. Plates were pre-incubated for 30 minutes at 37° C. before adding 5 μL 120 μM 4-MUH in 1:1 DMSO:Buffer A. Fluorescence was monitored every 5 minutes for 40 to 60 minutes to generate dose-response curves, from which IC₅₀ values were determined.

Kinetic Characterization of Inhibitors. To characterize potential lead compounds by inhibitor type, the turnover of 4-MUH (5-320 μM) was measured in the presence of 500 nM FAS TE. The actual Kᵢ values were calculated from the slopes at each inhibitor concentration:

$$slope = \frac{{K}_{m} ( 1 + \frac{[ I ]}{{K}_{i}} )}{{V}_{max}}$$

A replot of data from the reciprocal plot, Kₘ/Vₘₐₓ₍ᵢ₎ versus [I], distinguished pure and partial non-competitive inhibition. To establish reversibility of the inhibitors, a Vₘₐₓ versus [FAS TE] plot was generated. The reaction mixtures contained 10 μM inhibitor or vehicle (DMSO) with 45 μL of 500-1250 nM FAS TE in Buffer A or Buffer A alone. The final DMSO concentration did not exceed 10% (v/v). Plates were pre-incubated for 30 minutes at 37° C. before adding 5 to 320 μM 4-MUH in DMSO:Buffer A (1:1). The formation of fluorescent product was monitored in 5 minute intervals for 40 to 60 minutes.

Cell Culture. The MDA-MB-435 breast cancer cell line (Knowles et al., 2004; Menendez et al., 2004) was used as a model for the biological testing of the barbituric acid derivatives. MDA-MB-435 cells express FAS and undergo cell cycle arrest and apoptosis when FAS is inhibited, thereby providing a model platform. Cells were maintained in minimal Eagle's media, Earle's salts (Irvine Scientific) supplemented with 10% fetal bovine serum (Irvine Scientific), 2 mM L-glutamine (Invitrogen), minimal Eagle's media vitamins (Invitrogen), nonessential amino acids (Irvine Scientific) and antibiotics (Omega Scientific).

Testing Inhibitory Activity of Barbituric Acid Derivatives with an Activity-based Probe. Fluorescent labeling of the active site serine of the FAS TE was performed in cell lysates as described in Kridel et al. (2004) and Liu et al. (1999). Briefly, cells (5×10⁶) were resuspended in Buffer C (50 mM Tris-HCl, 150 mM NaCl, pH 8.0) on ice and lysed by sonication. Samples containing 50 μg total protein were incubated with various concentrations of test compounds or vehicle (DMSO, 0.1% v/v) on ice for 30 minutes. Fluorophosphonate (FP)-BODIPY probe (CombinX) was added to samples at a final concentration of 50 nM and incubated at room temperature for 30 minutes. The reaction was stopped by the addition of 5× SDS loading buffer (124 mM Tris, pH 8.3, 959 mM glycine, 17 mM SDS). Samples were analyzed by SDS-PAGE electrophoresis on a 10% Tris-glycine Criterion gel (Bio-Rad) at 200 V for 60 minutes and visualized on a Hitachi flatbed scanner at 505 nm.

Measuring Fatty Acid Synthesis in vitro. Fatty acid synthesis by the FAS holoenzyme in cell lysates was measured by incorporation of [¹⁴C] malonyl-CoA (Amersham). MDA-MB-435 cells (5×10⁶ total) were lysed by sonication in Buffer B (20 mM Tris-HCl pH 7.5, 1 mM EDTA, 1 mM DTT). Each reaction contained 100 μg total cellular protein and 5 to 50 μM of inhibitor or vehicle (DMSO, 10% v/v) as a control. Samples were incubated on ice for 60 minutes prior to addition of reaction mixture (130 μL; 115 mM KCl, 192.2 μM acetyl-CoA, 577 μM NADPH) and [¹⁴C] malonyl-CoA (5 μL; 0.1 μCi). Samples were incubated at room temperature for 2 hours and fatty acids were extracted with chloroform:methanol (1:1). The chloroform fractions were dried overnight and, re-extracted with hydrated butanol:water (1:1). The butanol fractions were reduced to 400 μL under nitrogen, and added to EcoLume (ICN Biomedicals) scintillation fluid (3 mL). Labeled fatty acids were detected by scintillation. All samples were prepared in duplicate.

Measuring Cytotoxicity. For cytotoxicity experiments, MB-MDA-435 cells were plated in 96-well plates at 1.2×10⁴ cells/well in complete MEM (200 μL) and incubated overnight at 37° C. and 5% CO₂. Cells were treated with test compounds (12.5 to 100 μM) or vehicle in triplicate, with a final percentage of DMSO not exceeding 1% (v/v). At 48 hours, the medium was aspirated and replaced with complete MEM, containing 333 μg/mL [3-(4,5-dimethylthiazol-2-yl)-5-(3-carboxymethoxyphenyl)-2-(4-sulfophenyl)-2H-tetrazolium (MTS) and 25 μM phenazine methosulfate (PMS), using the CellTiter 96 AQᵤₑₒᵤₛ Non-Radioactive Cell Proliferation Assay (Promega). Plates were incubated for 2 hours and absorbance was assayed at 490 nm. Background levels of formazan formation were measured in medium alone. IC₅₀ values were derived from dose-response curves.

Results

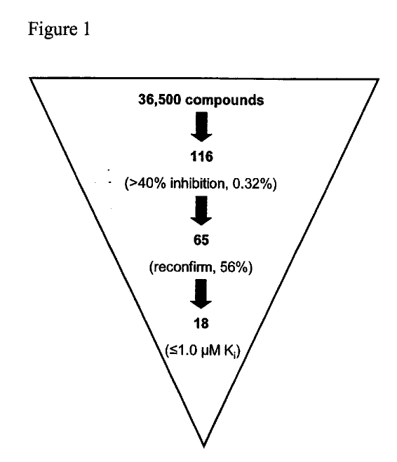

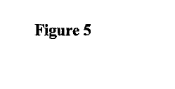

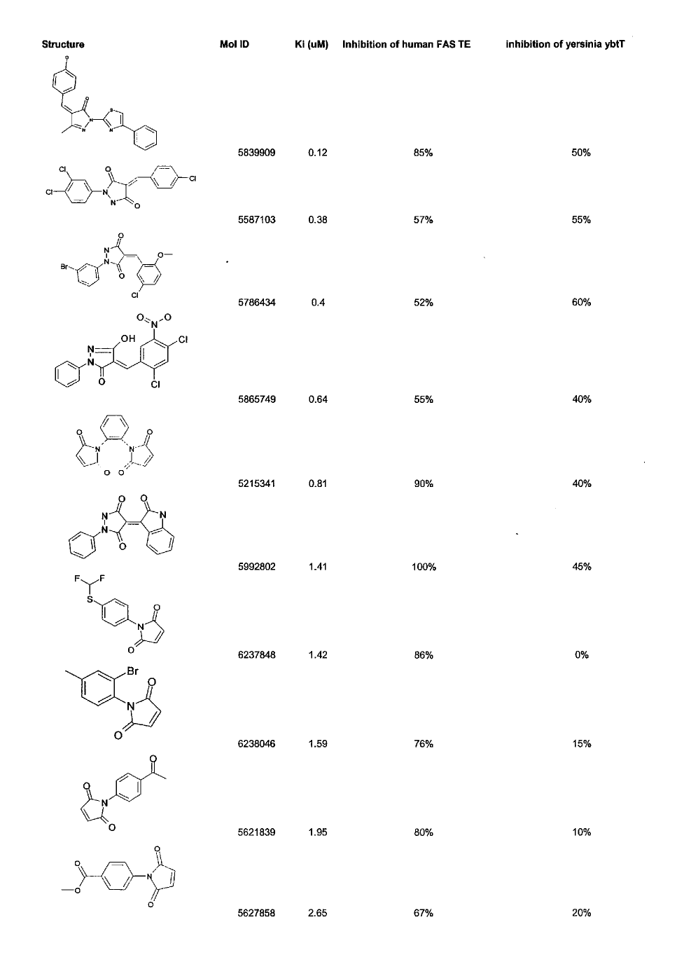

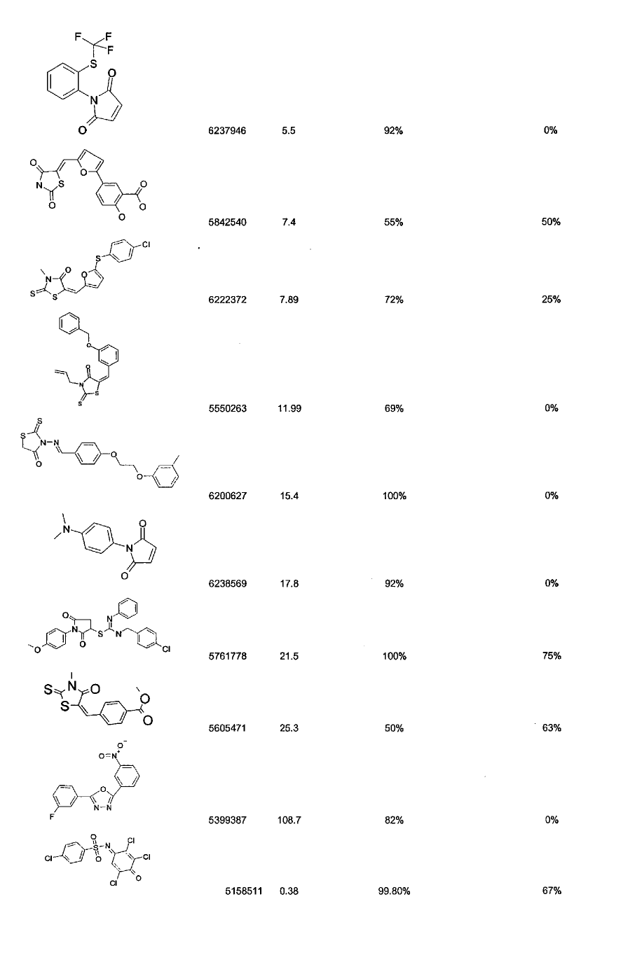

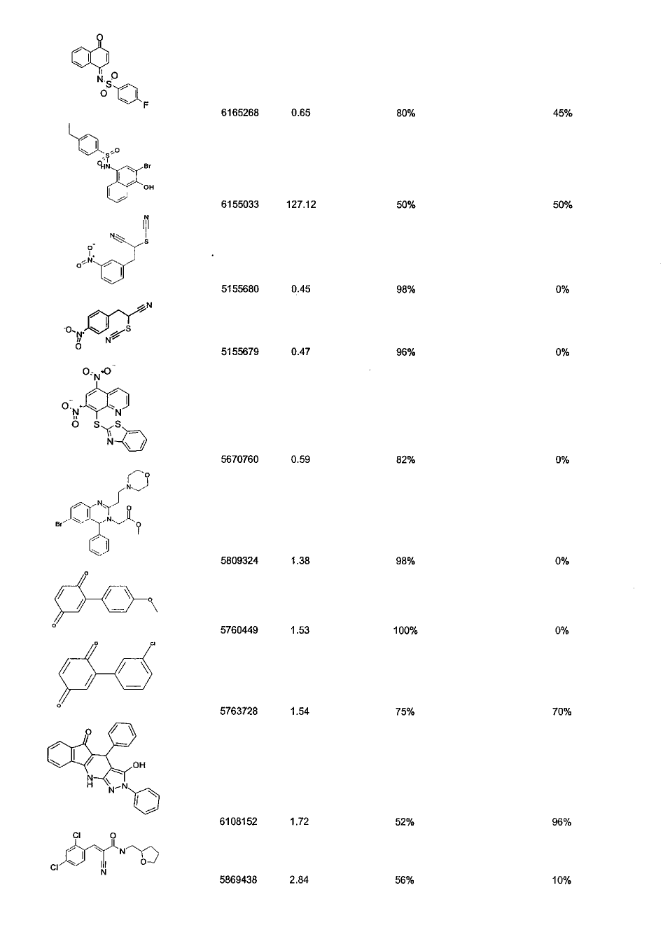

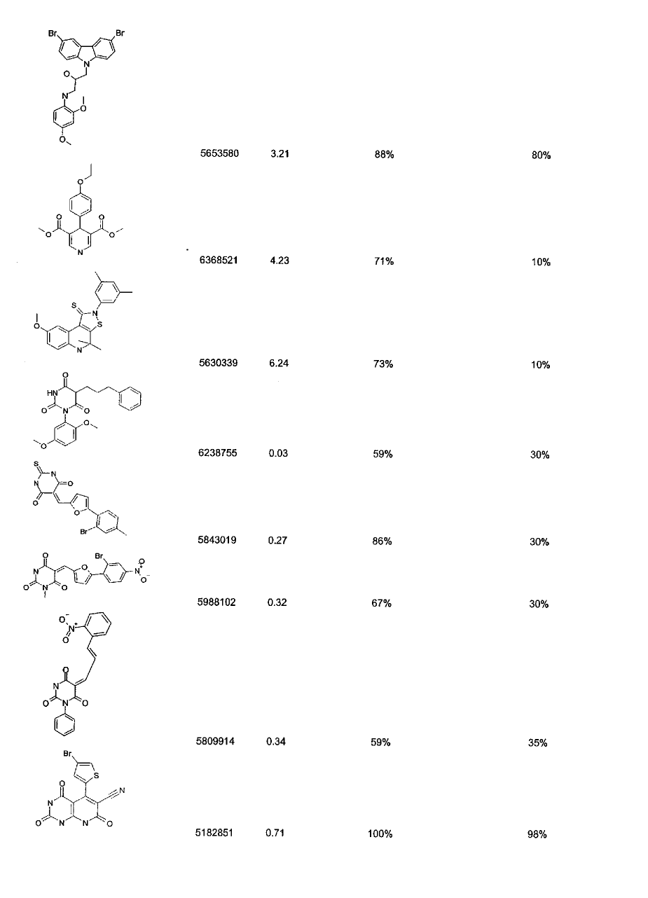

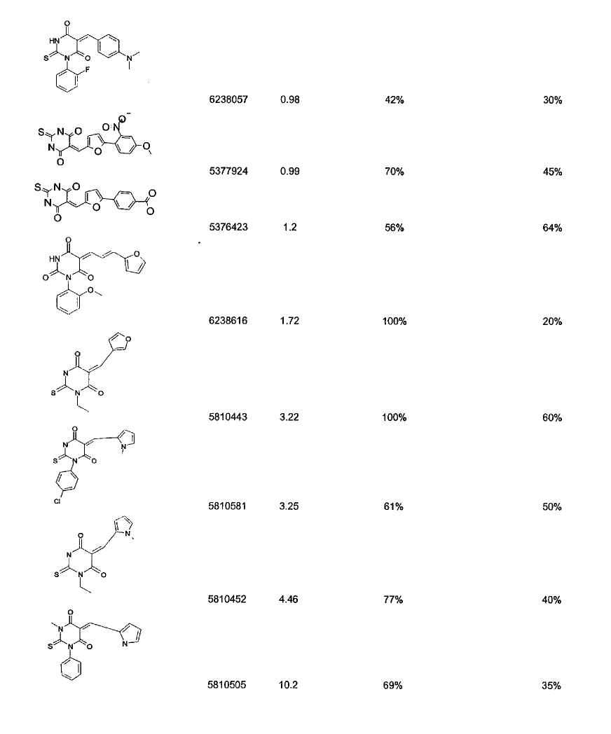

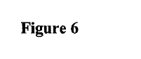

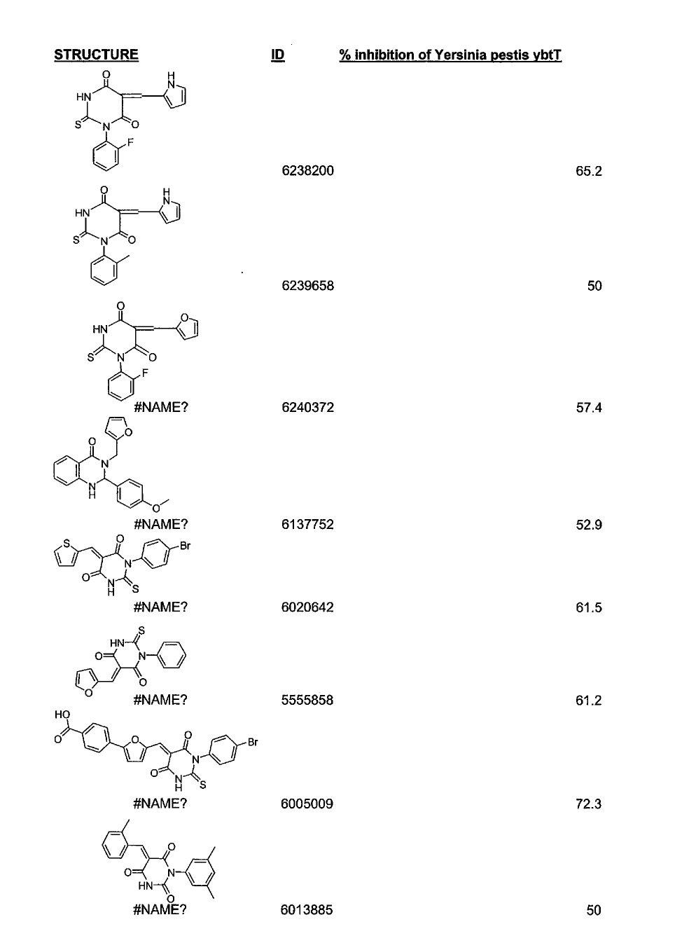

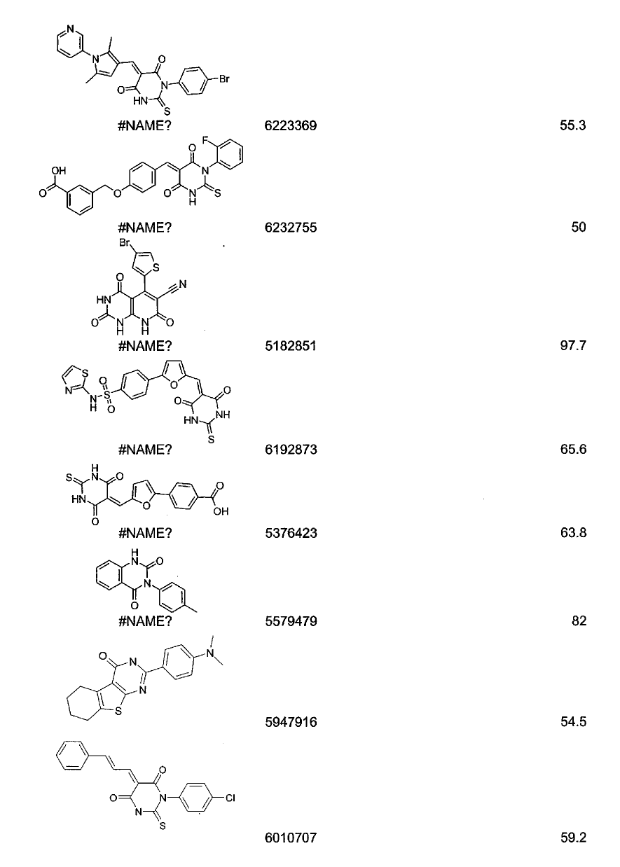

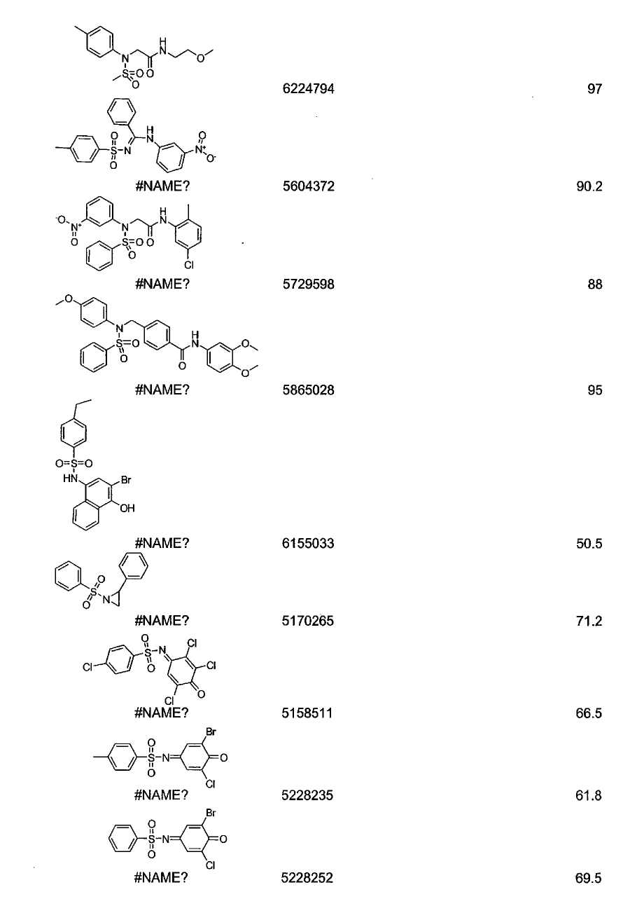

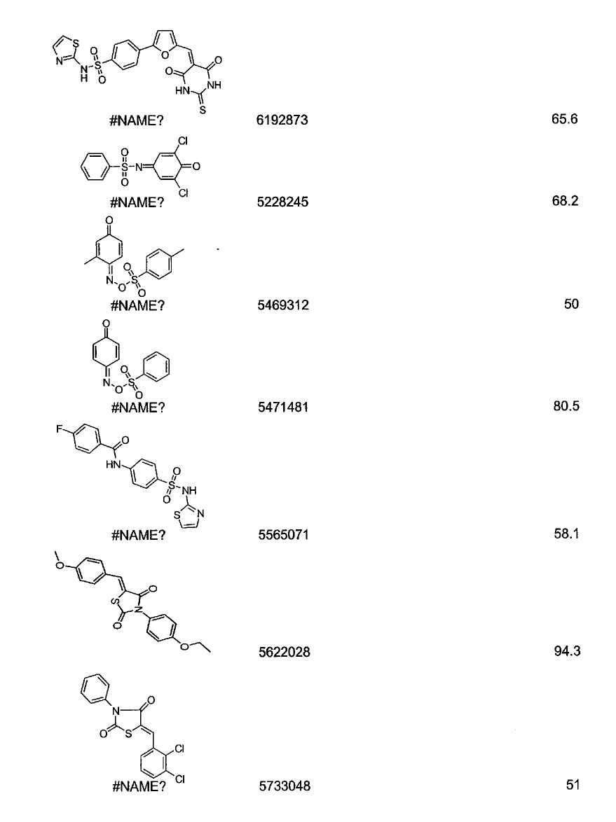

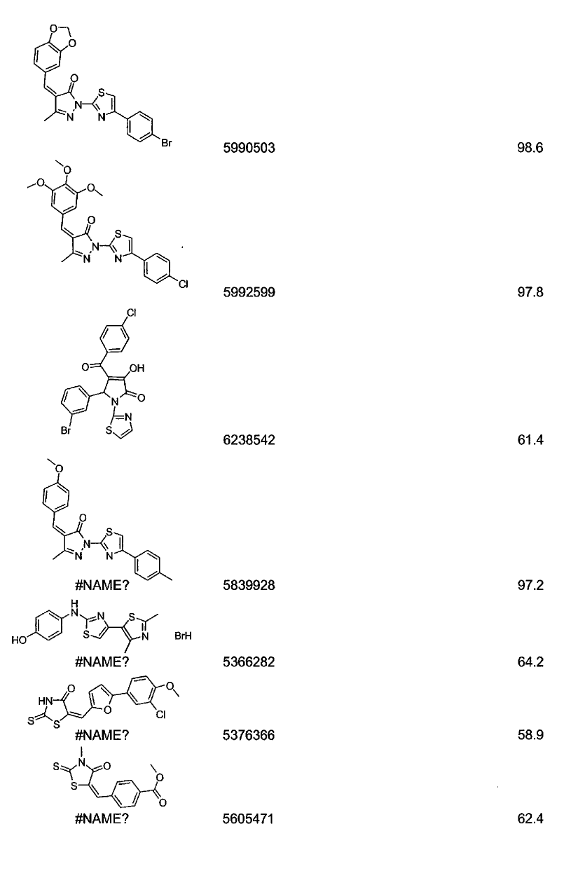

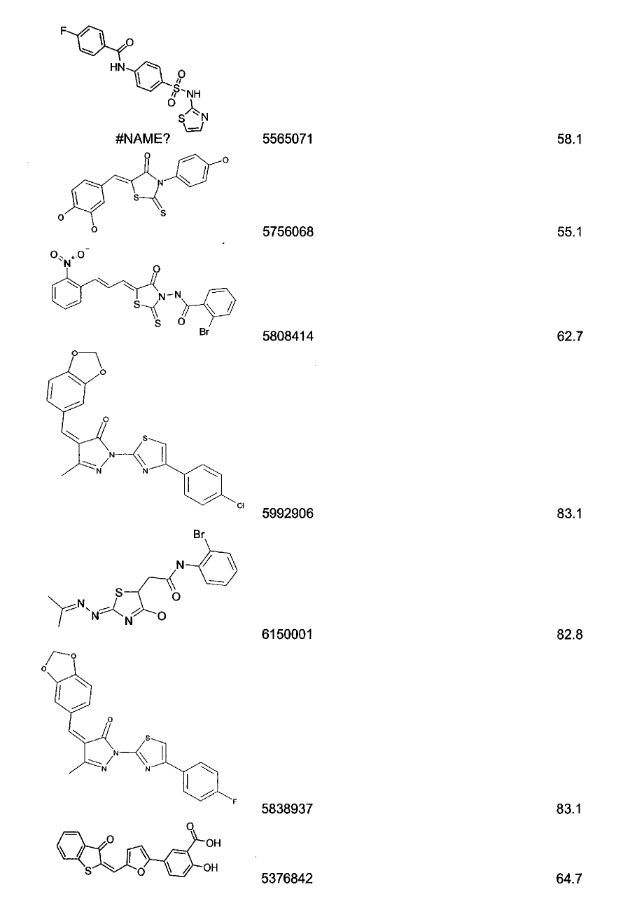

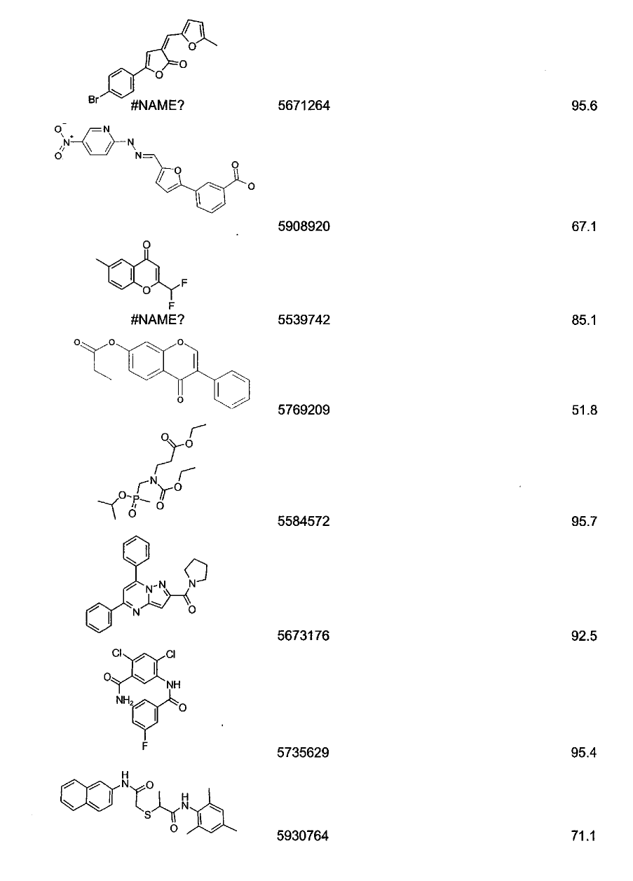

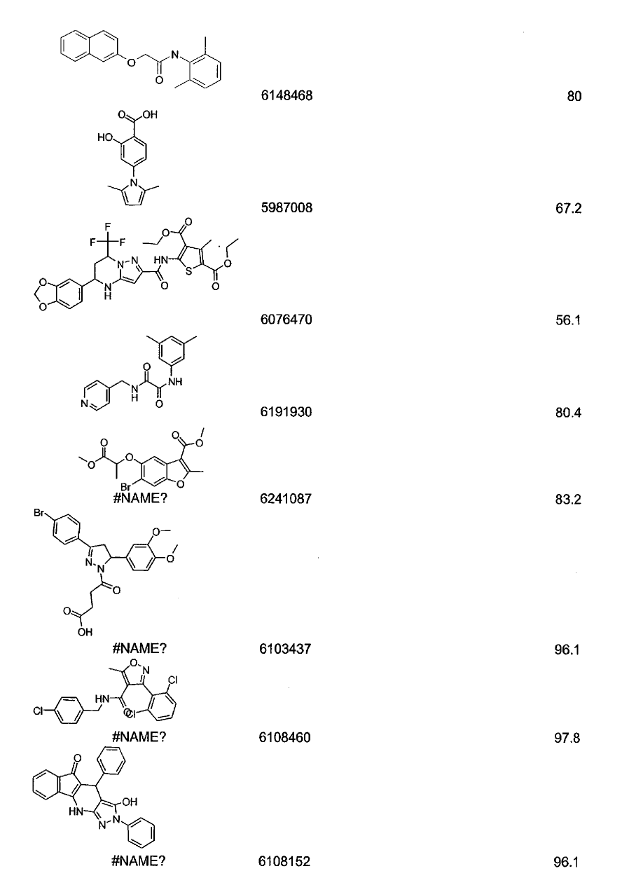

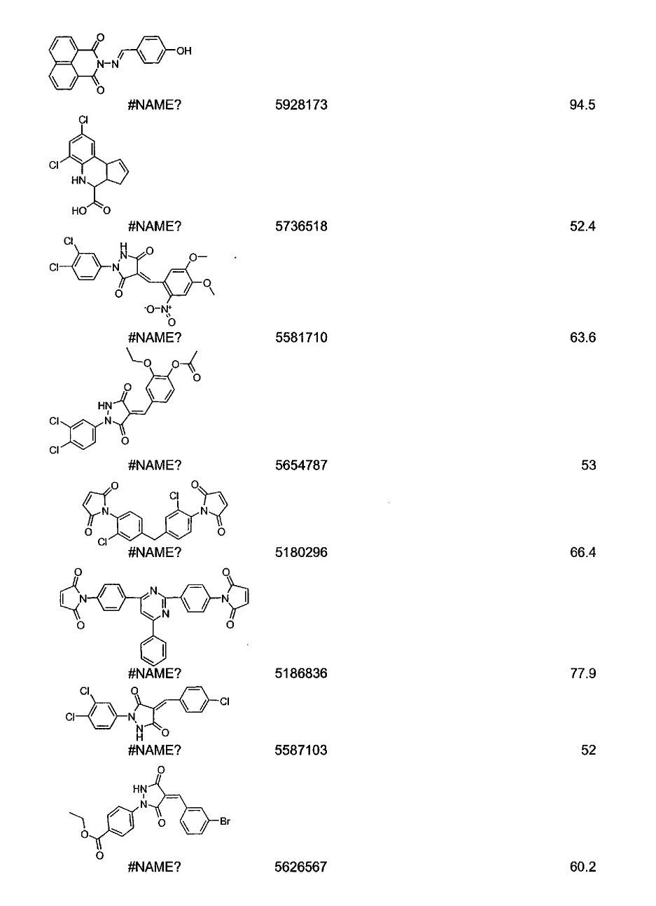

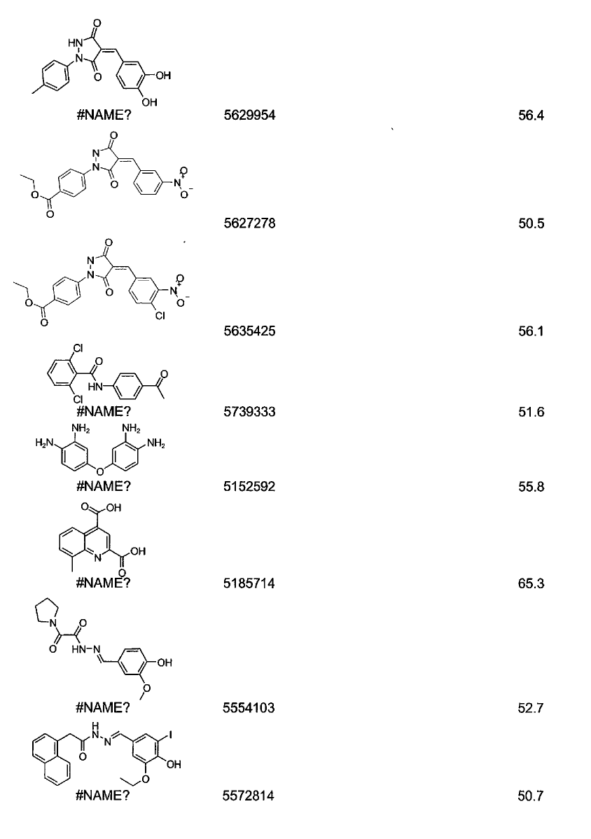

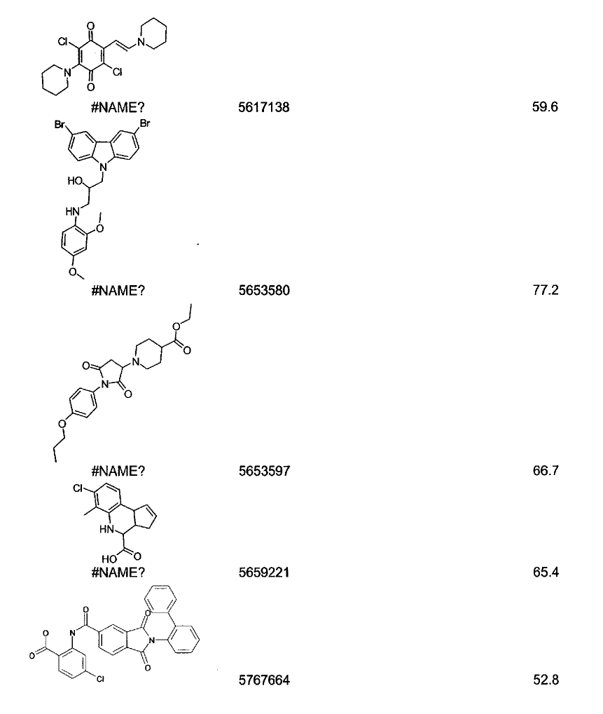

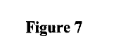

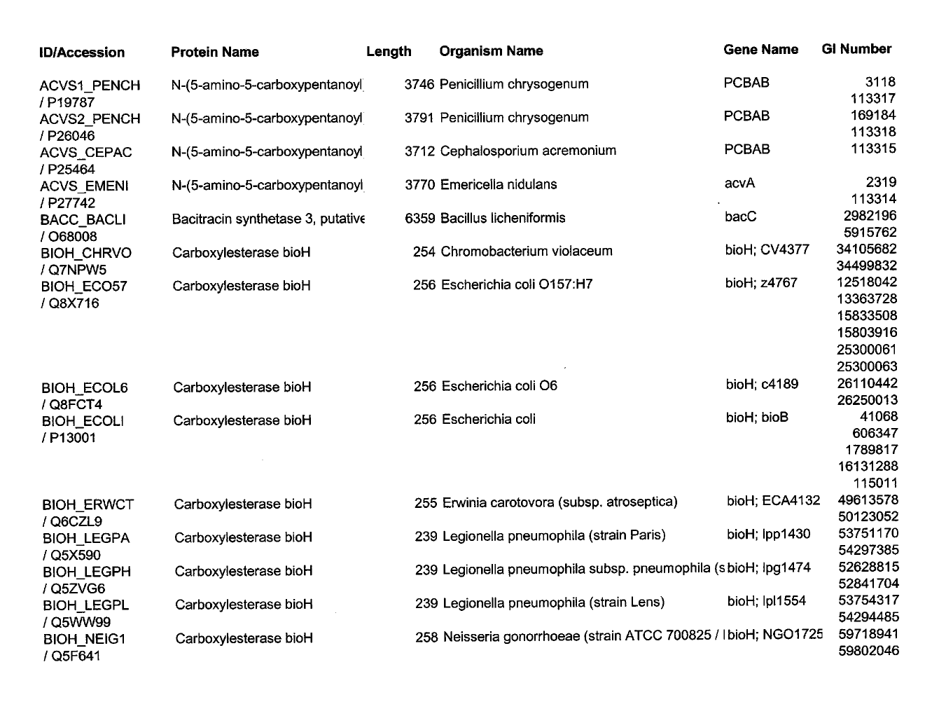

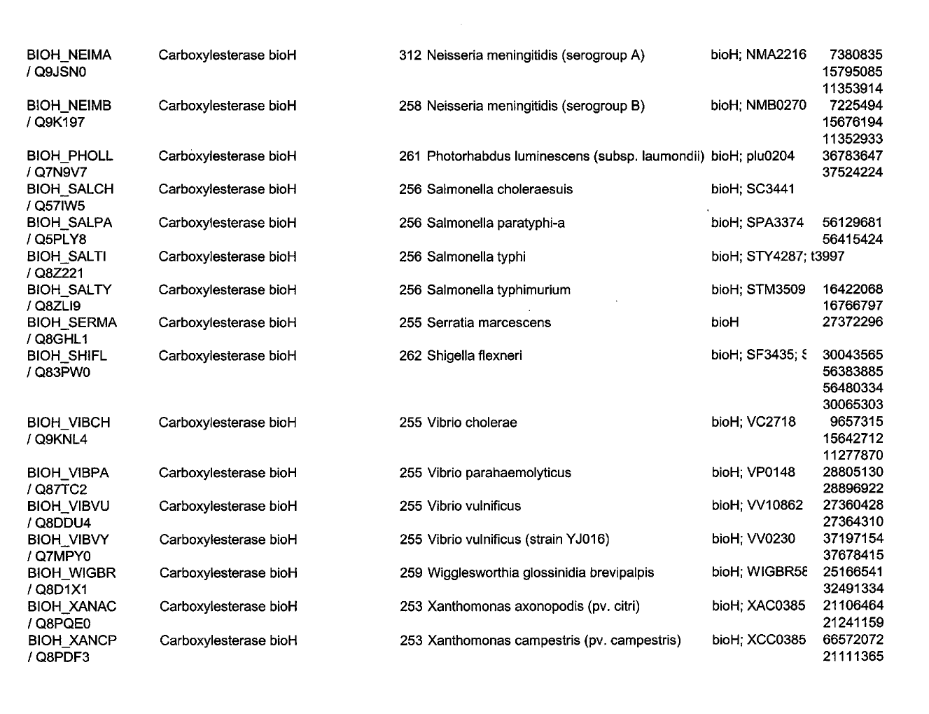

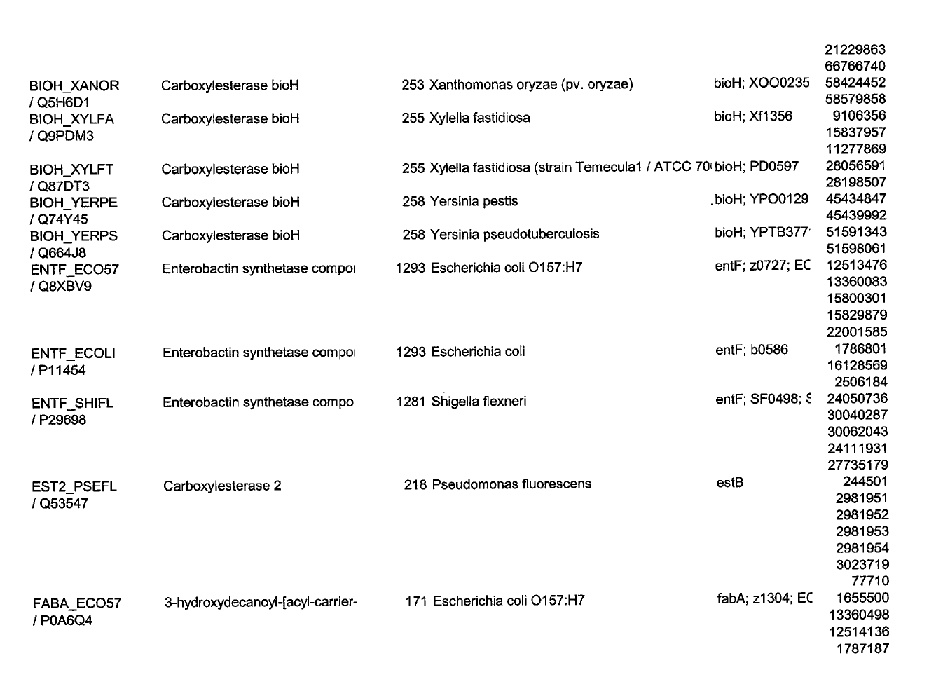

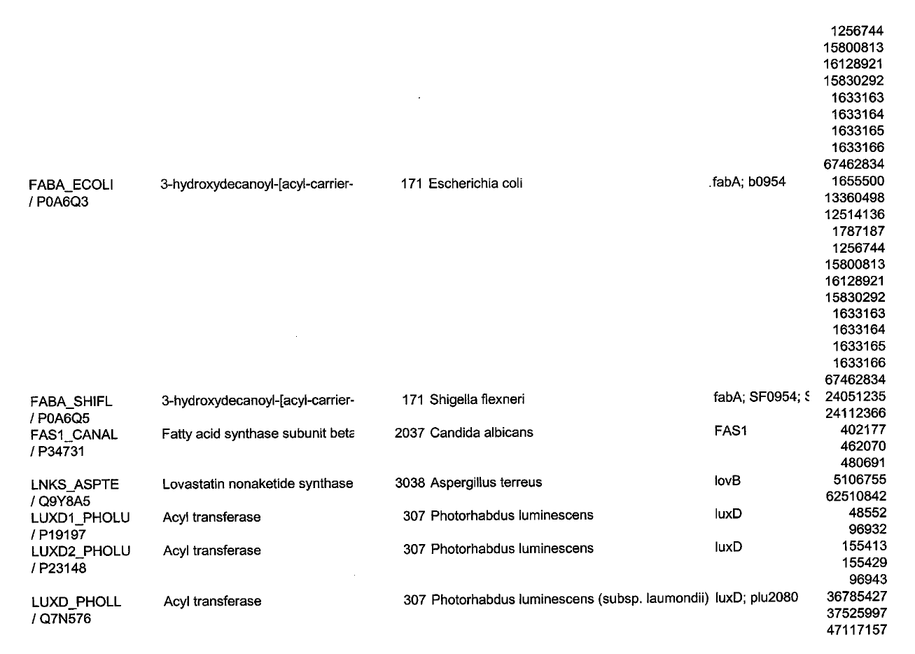

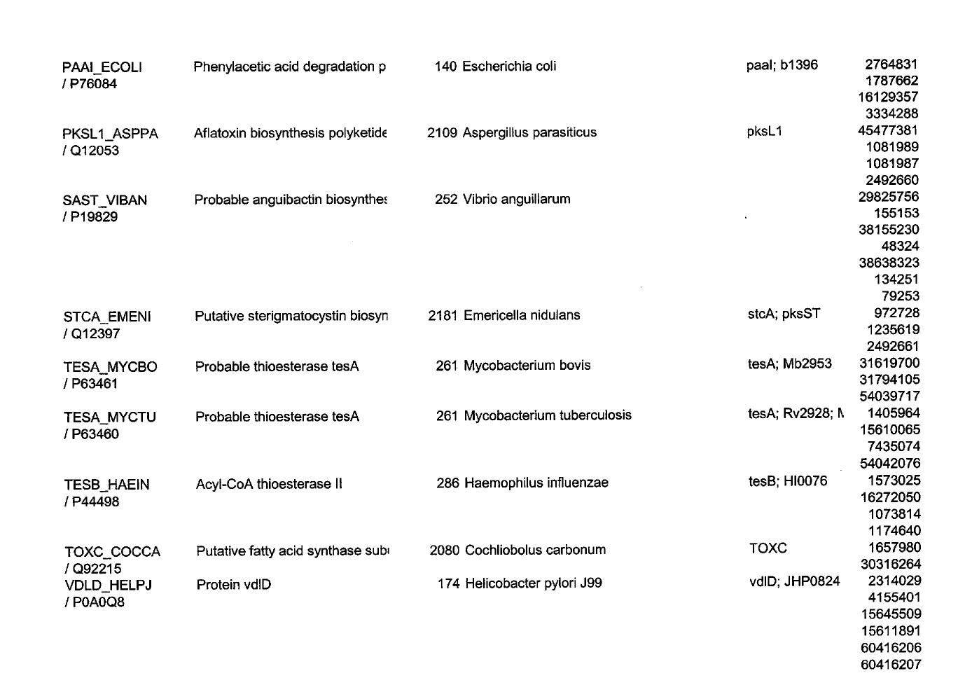

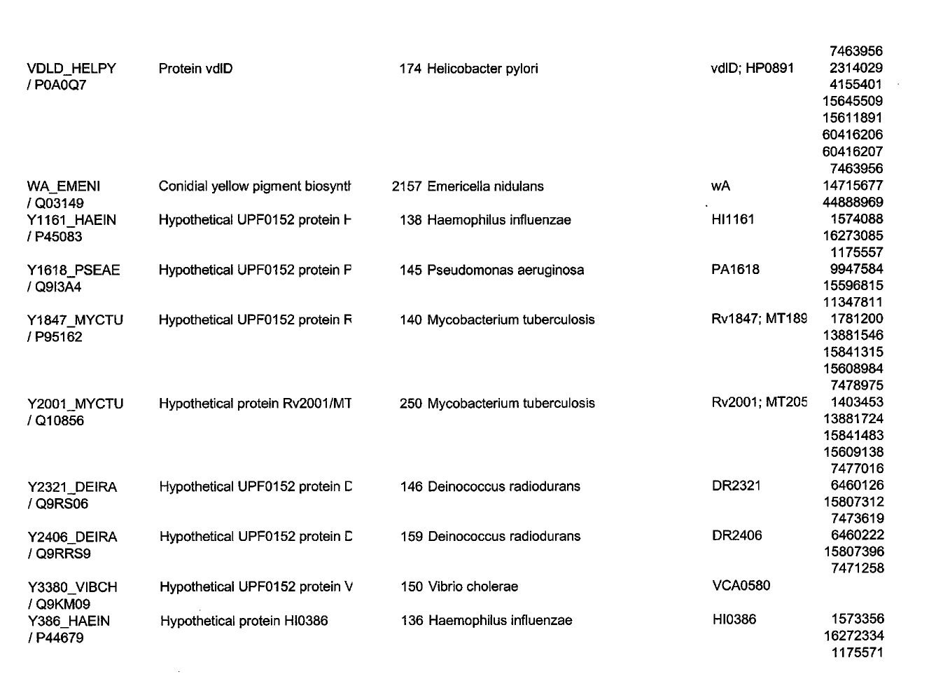

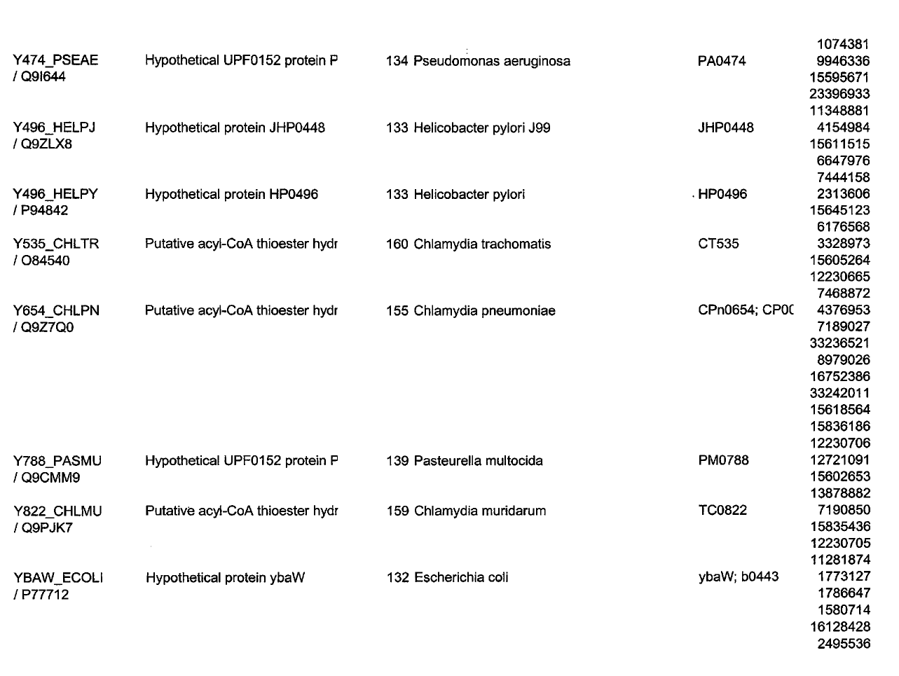

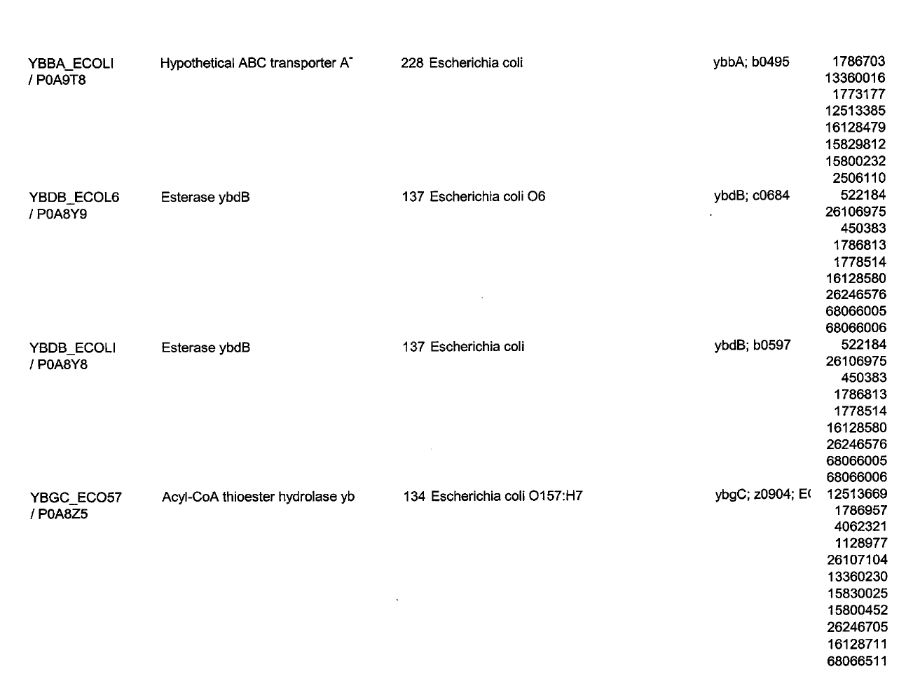

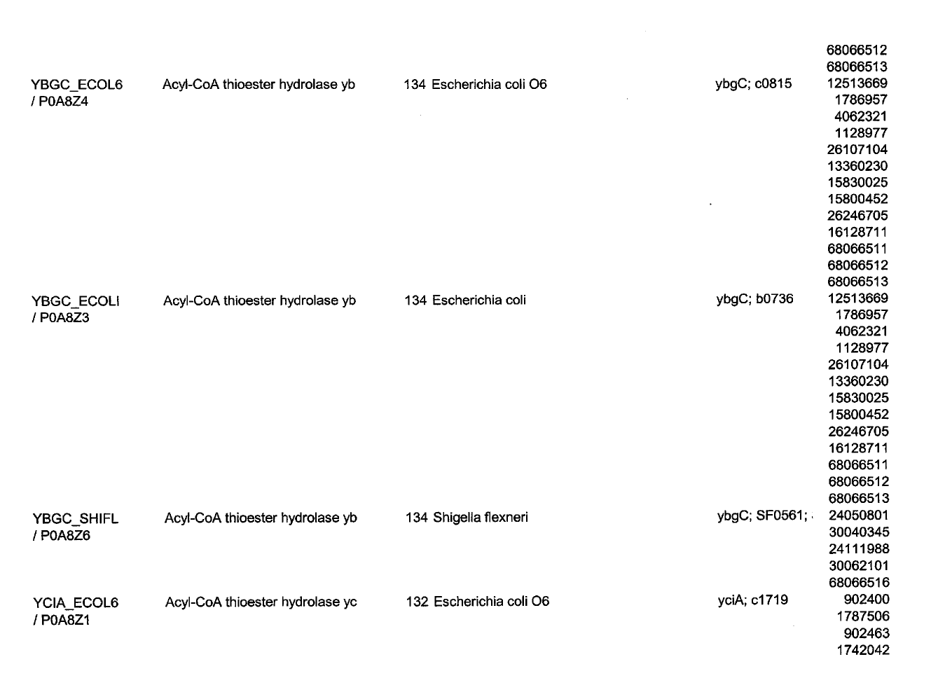

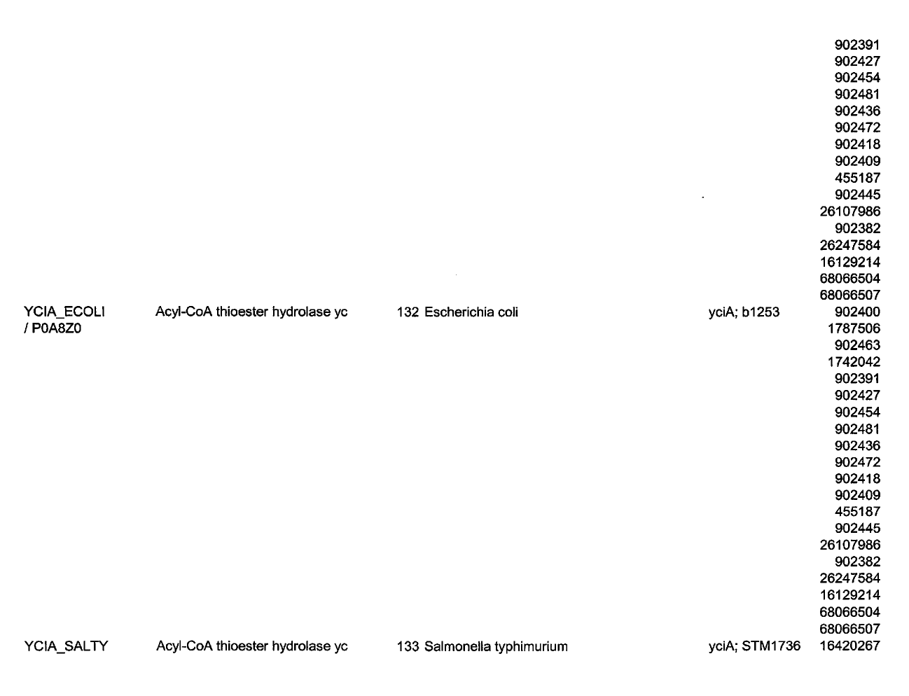

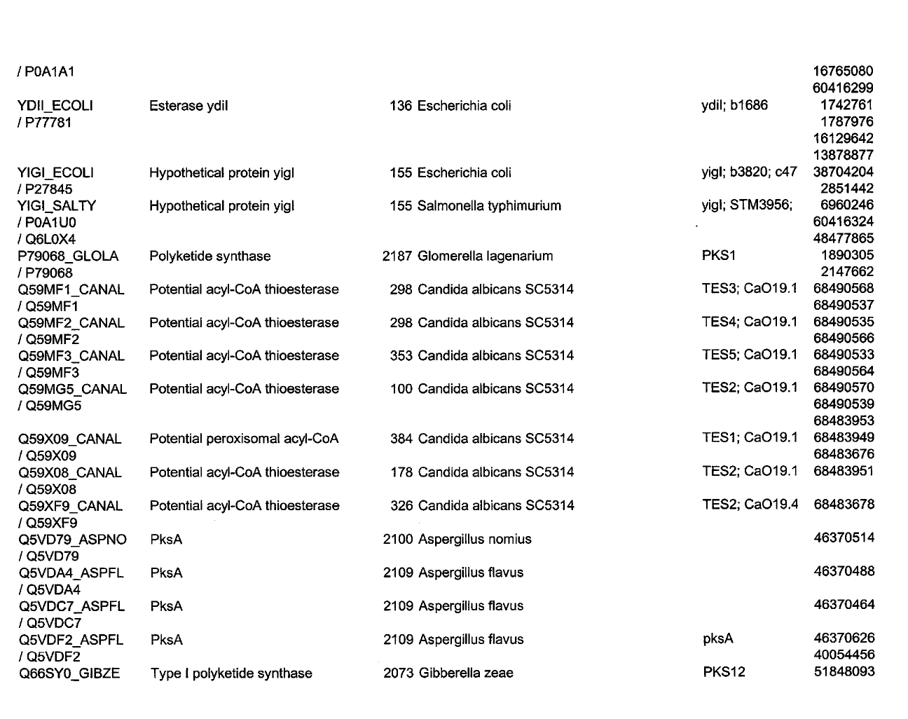

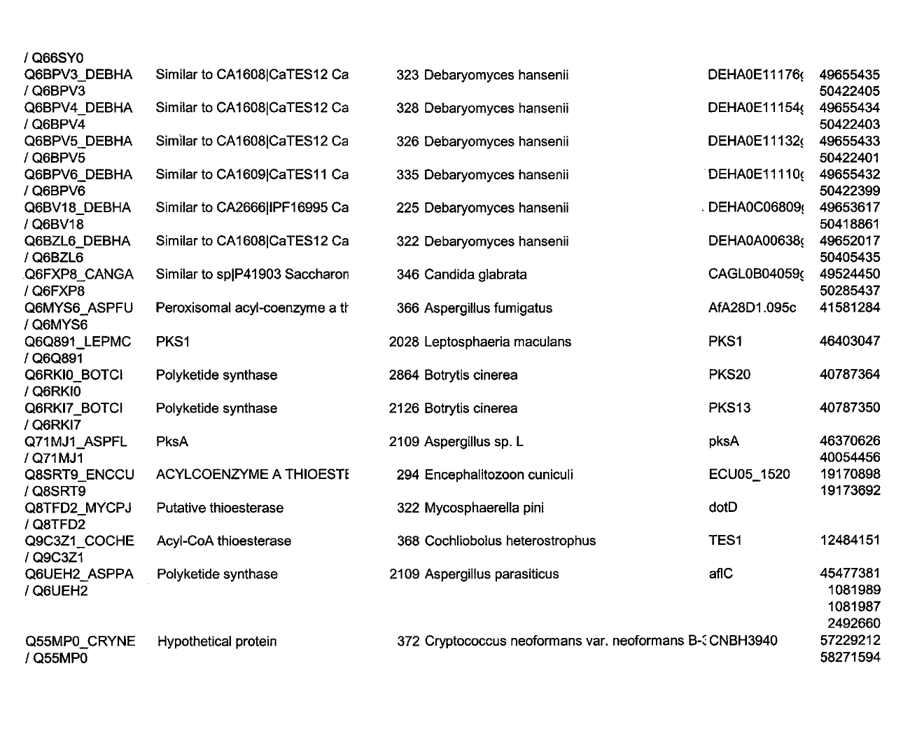

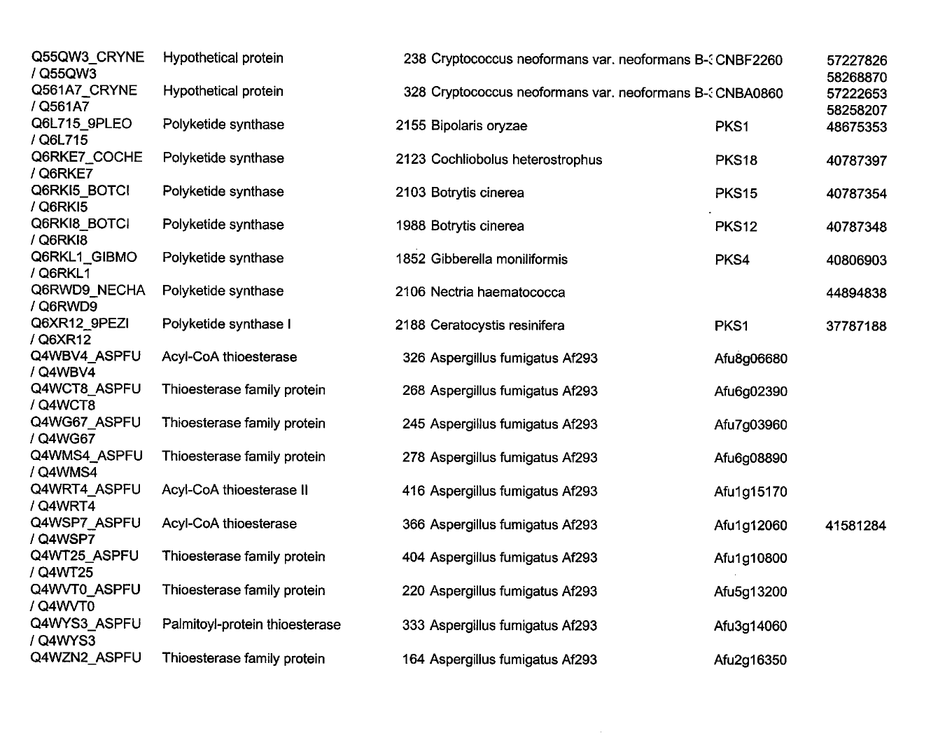

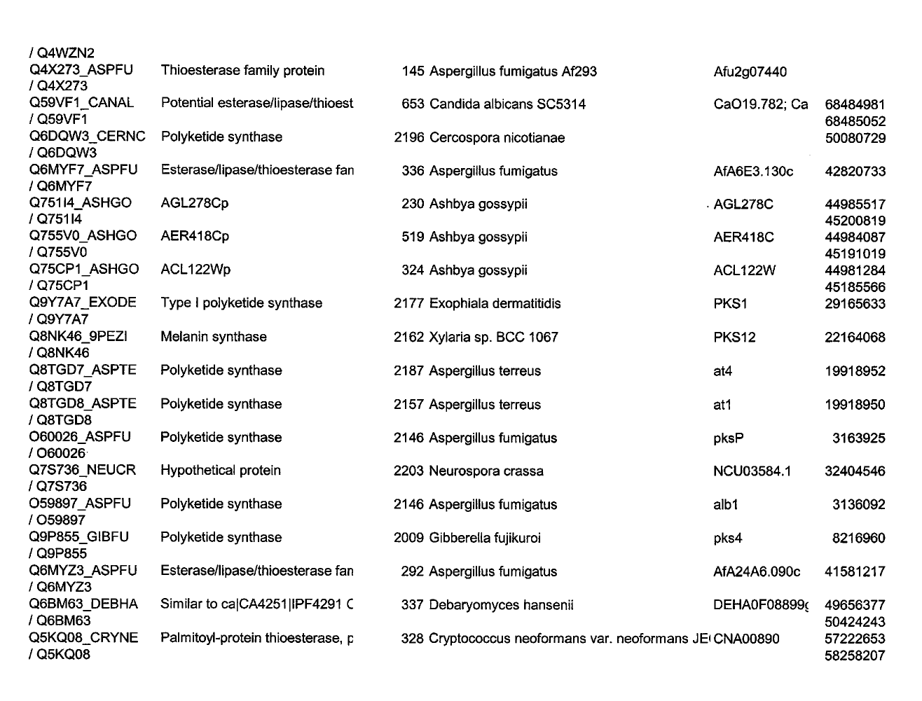

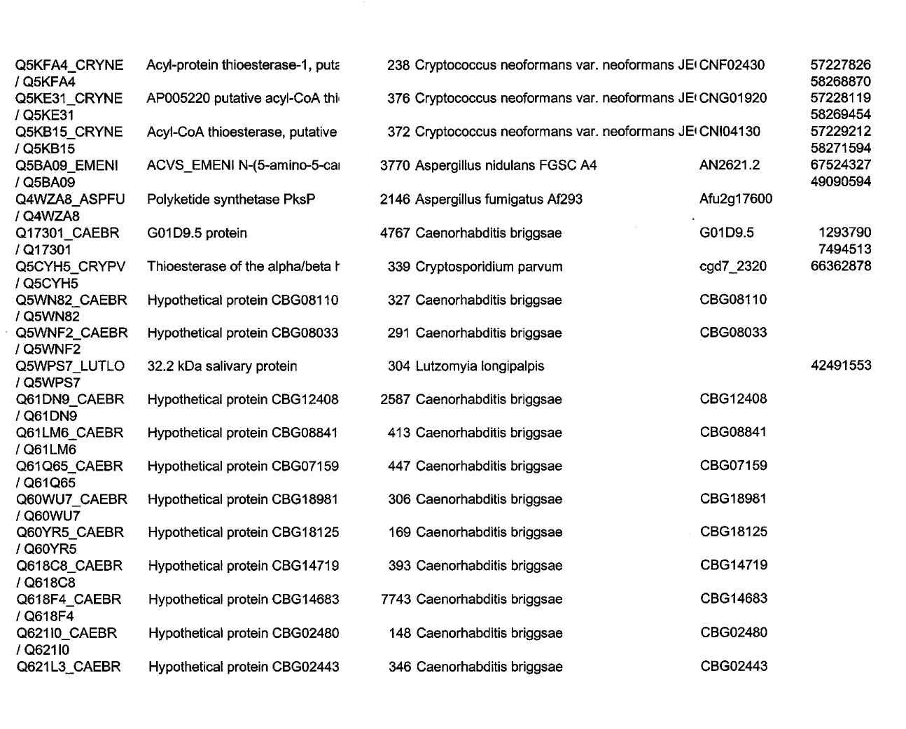

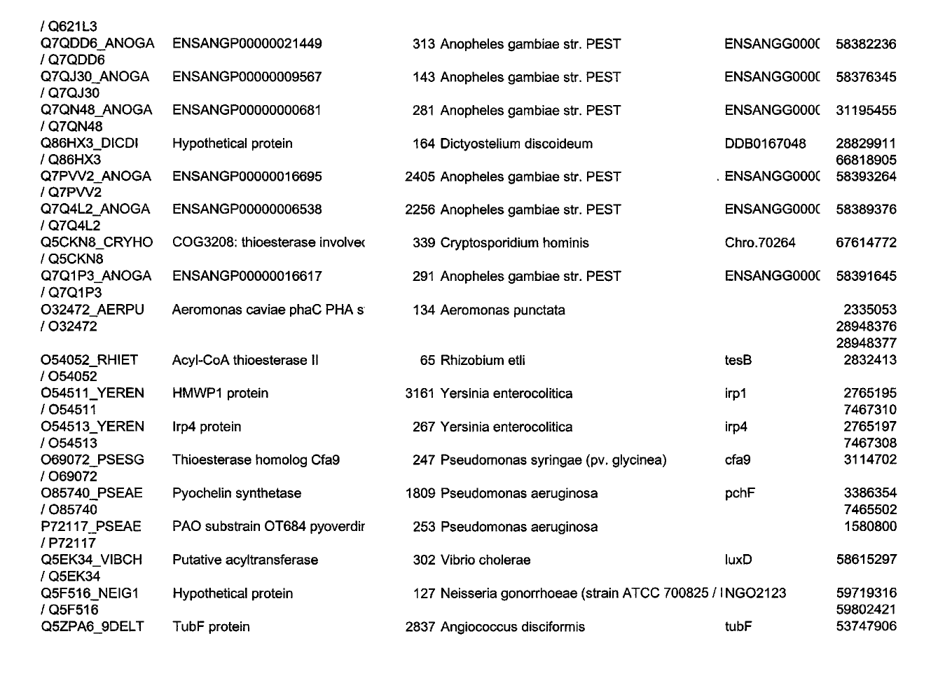

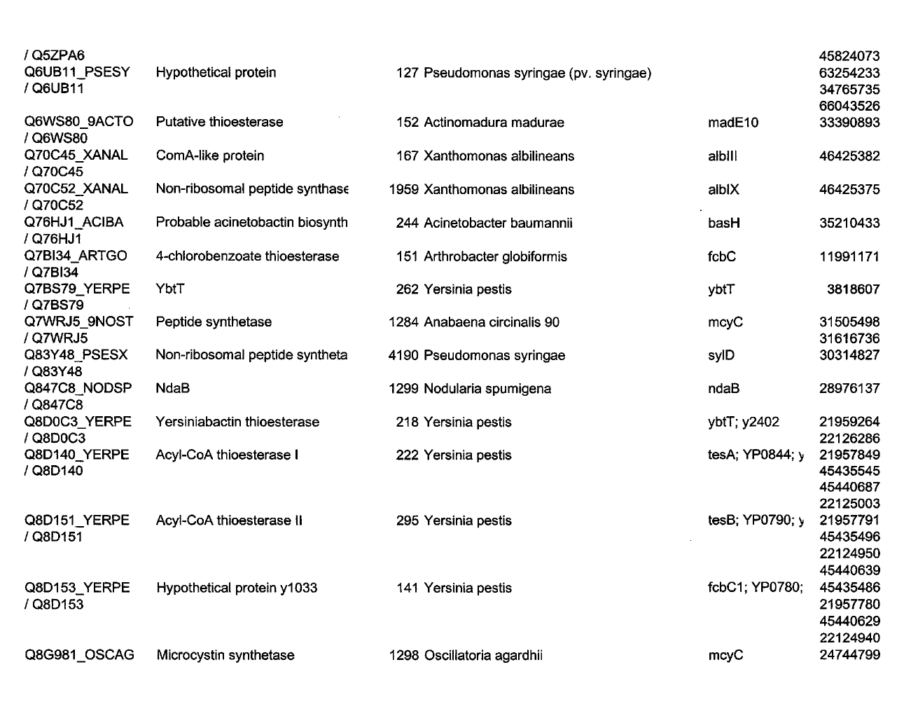

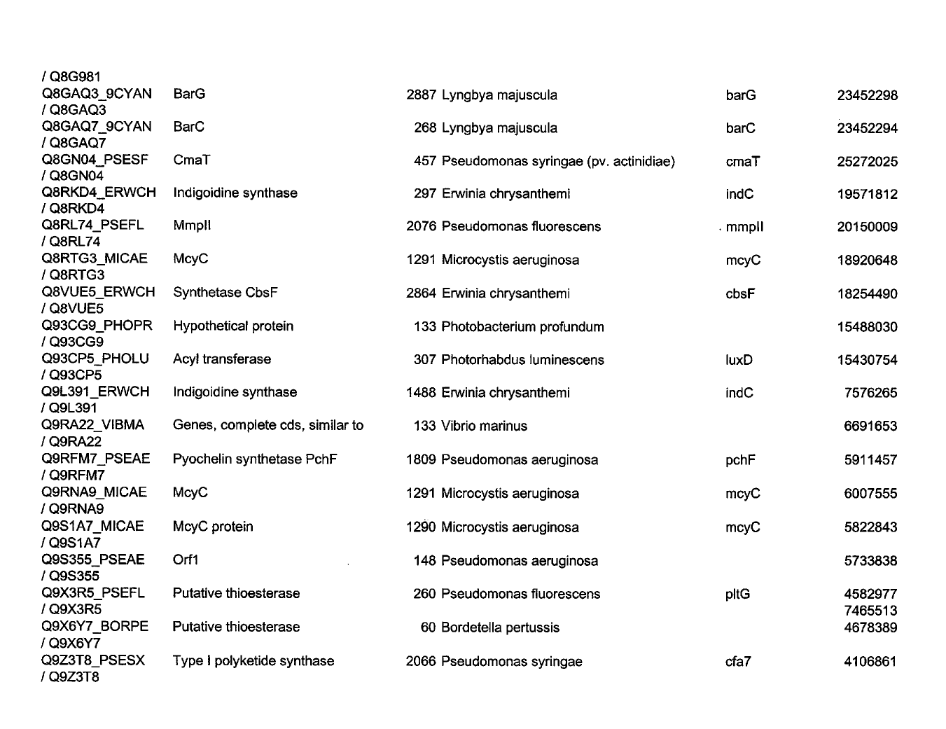

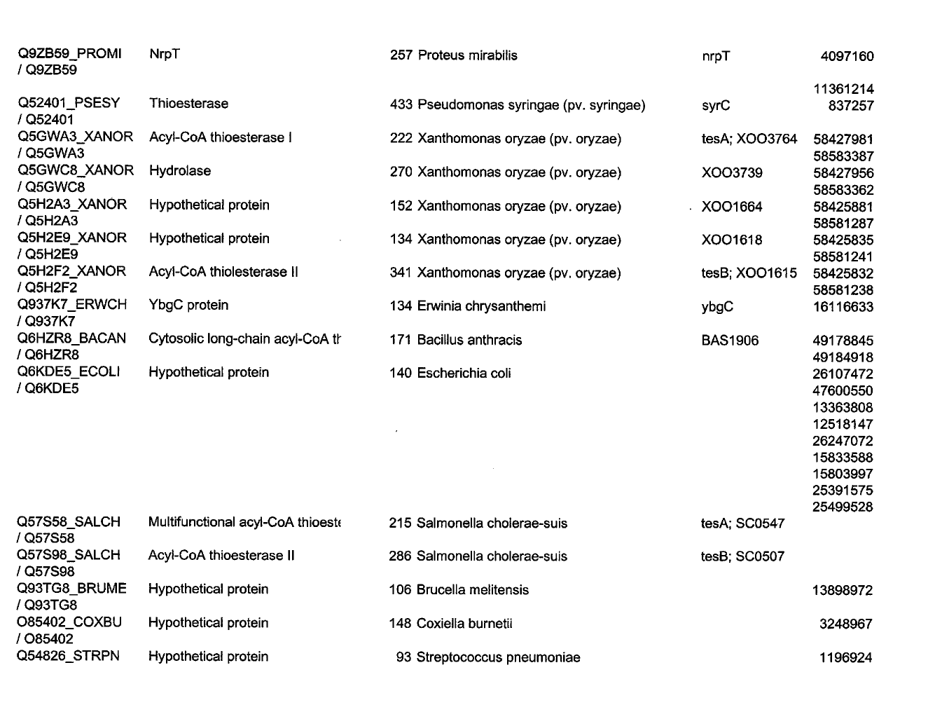

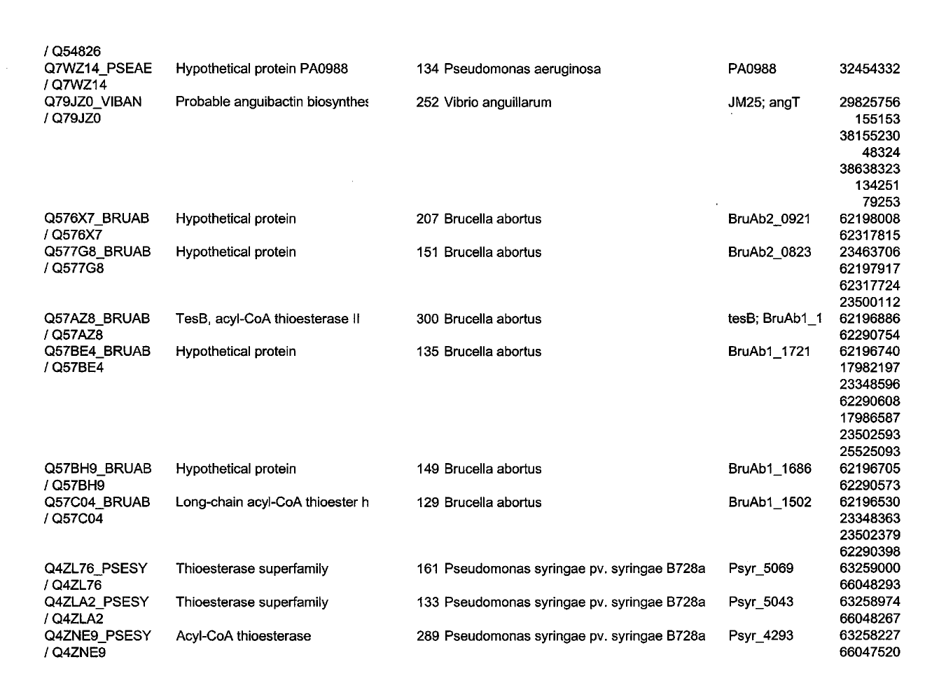

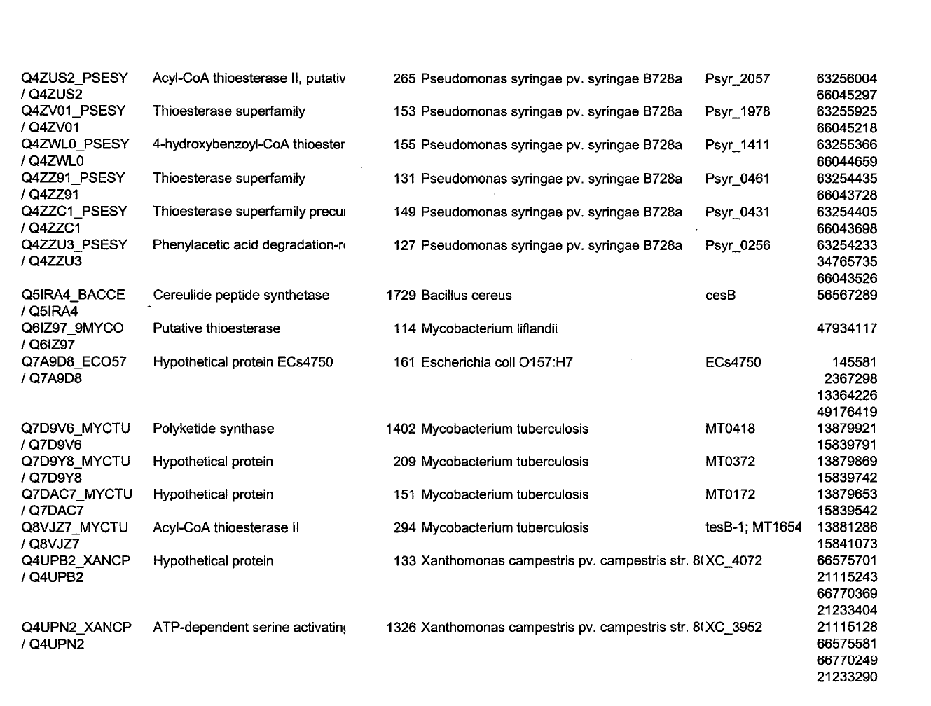

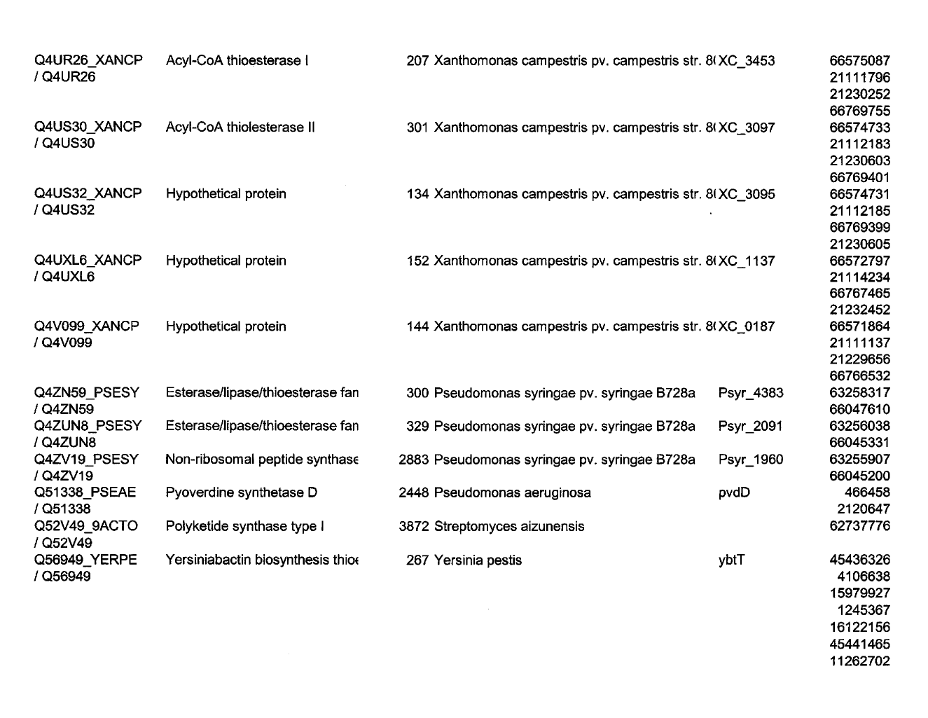

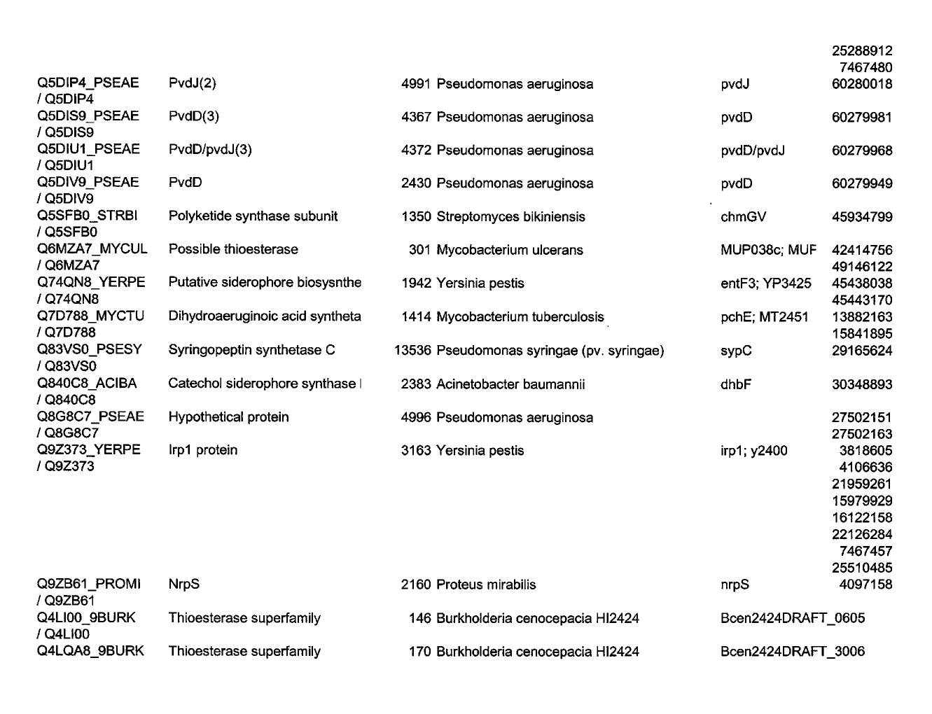

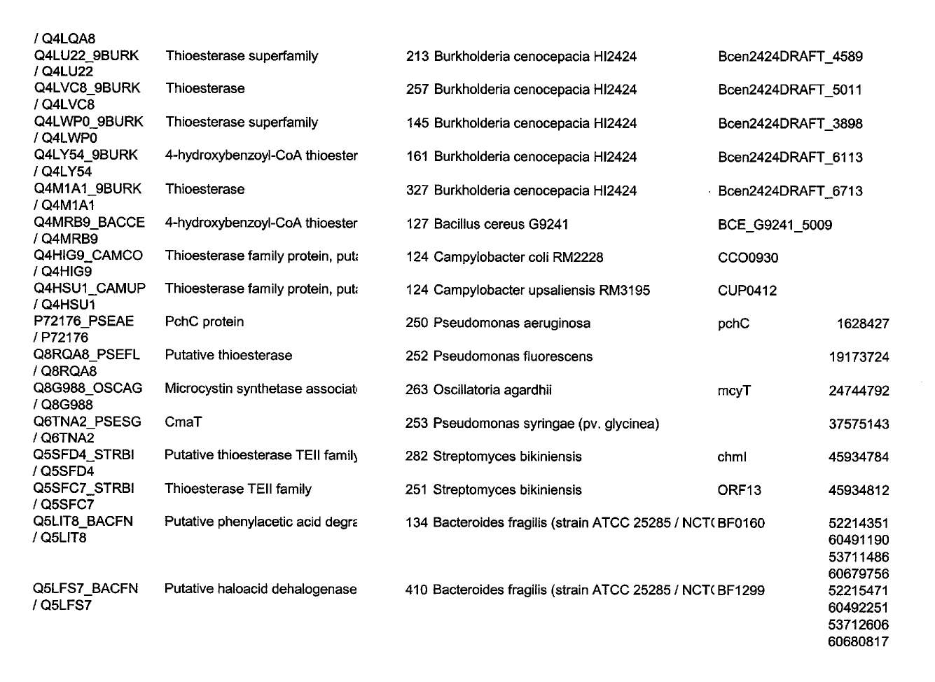

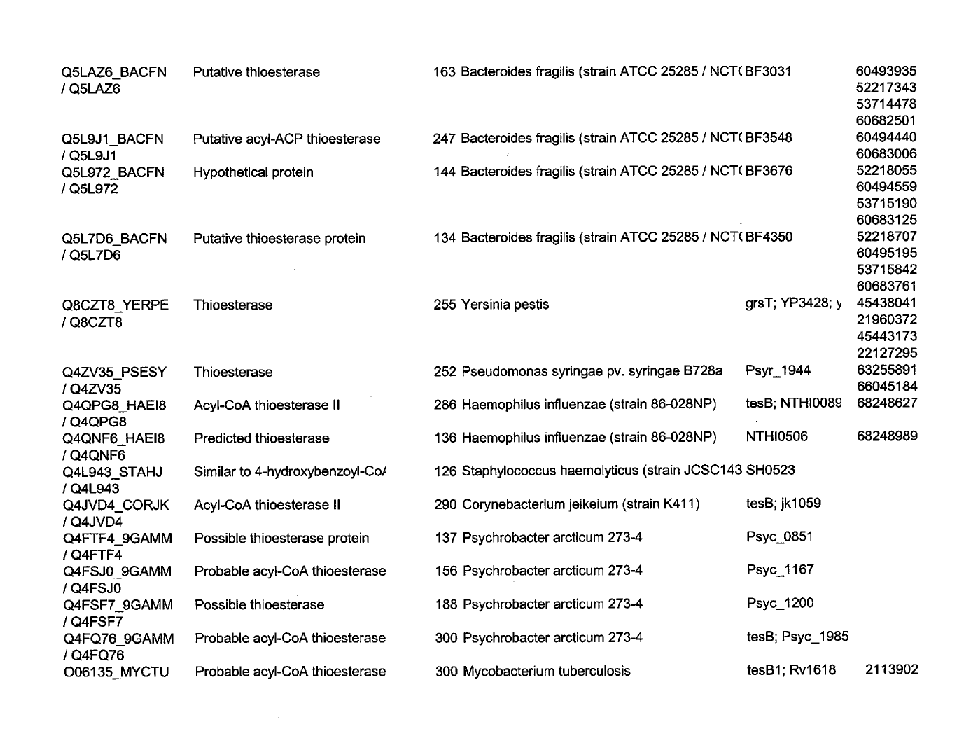

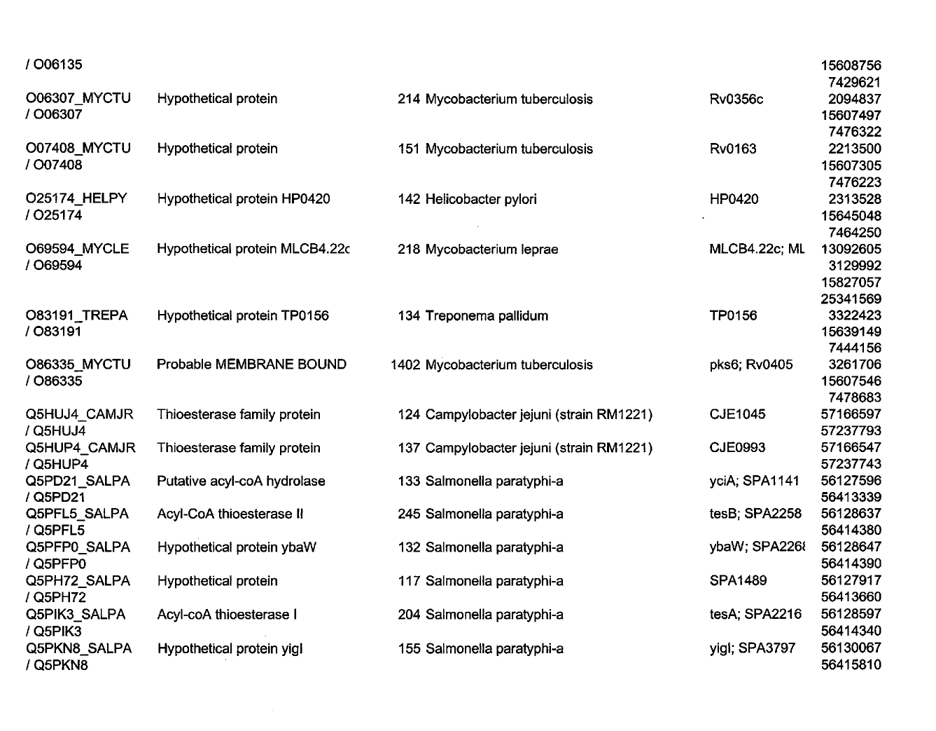

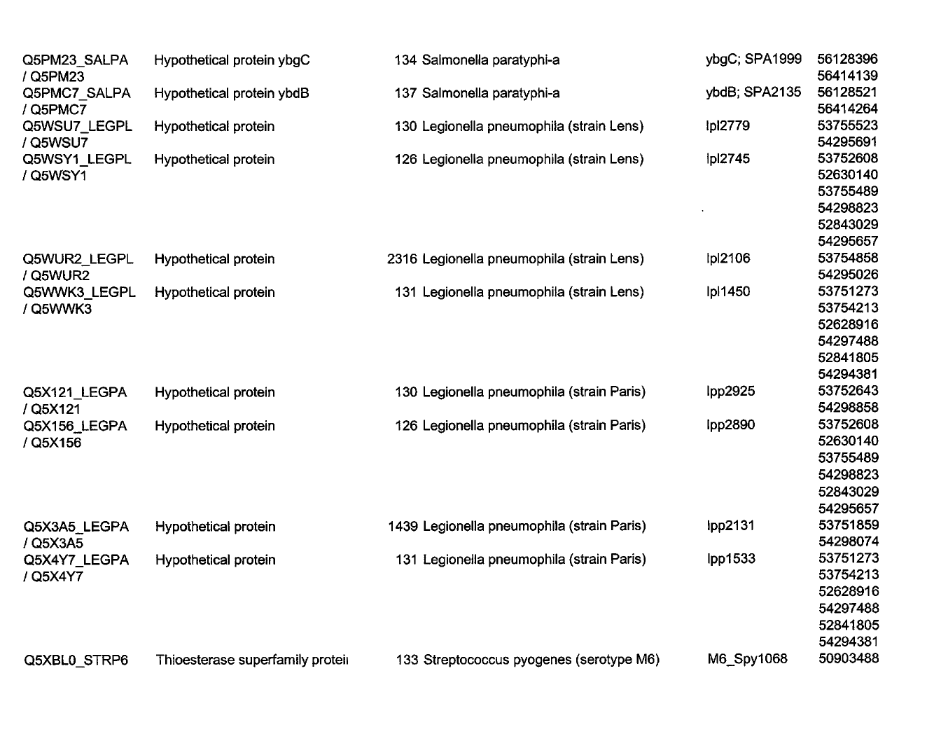

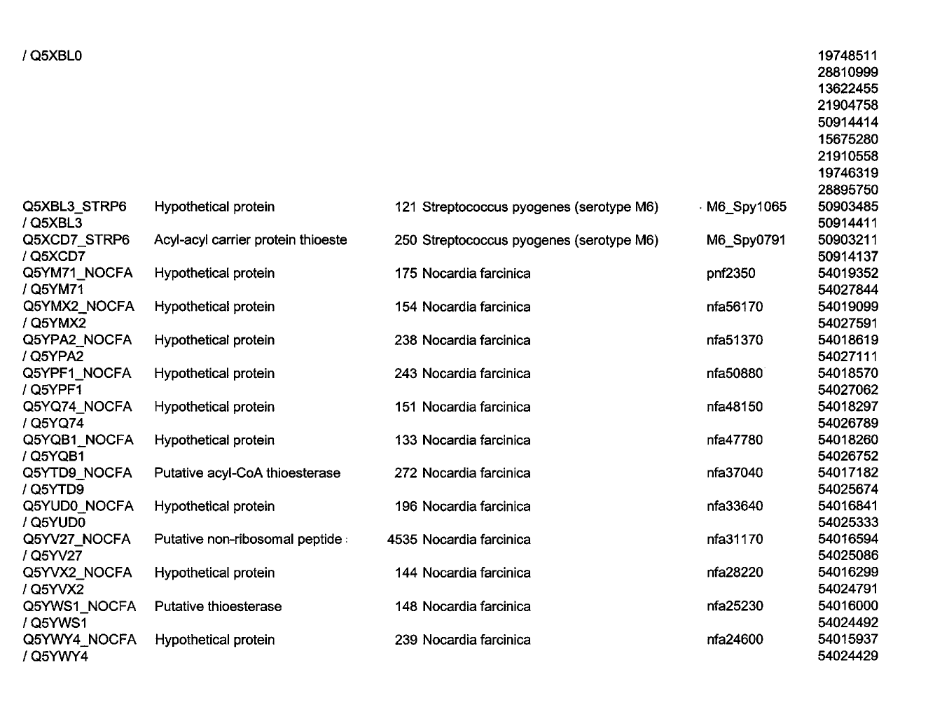

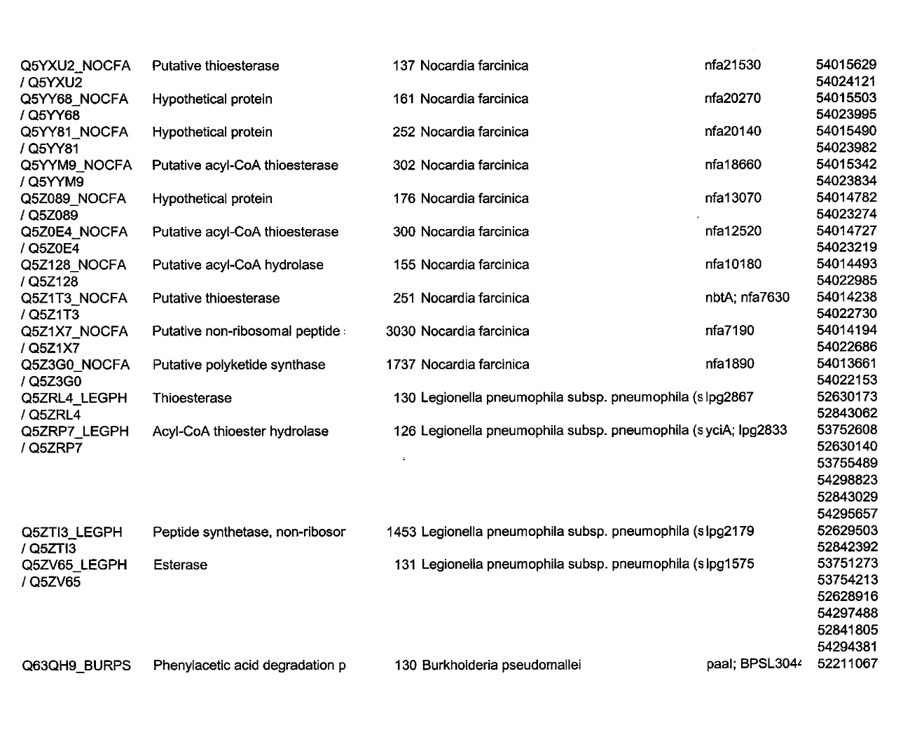

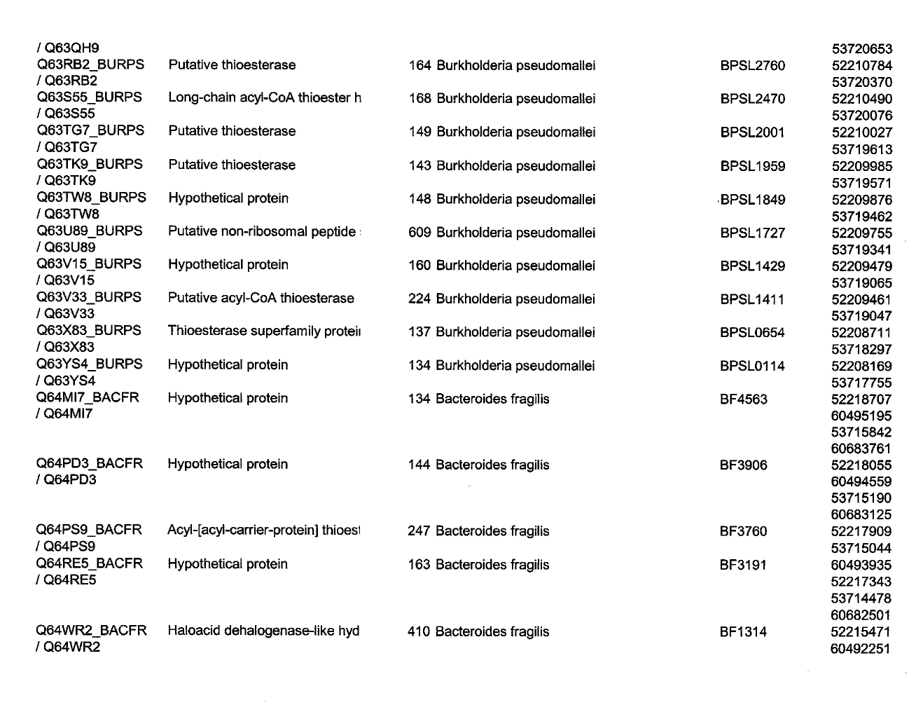

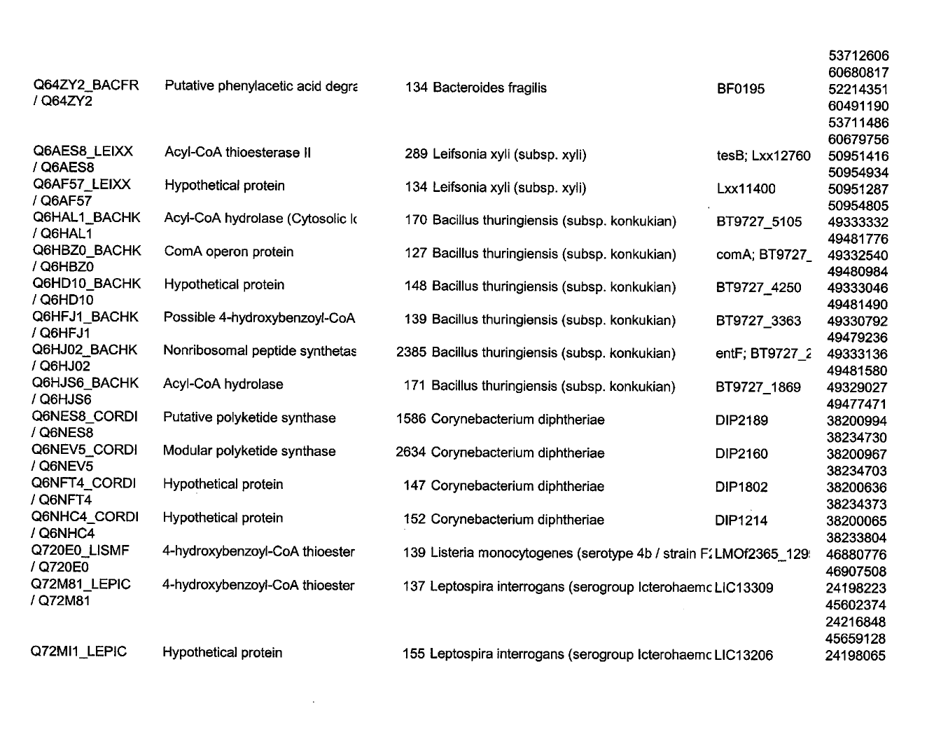

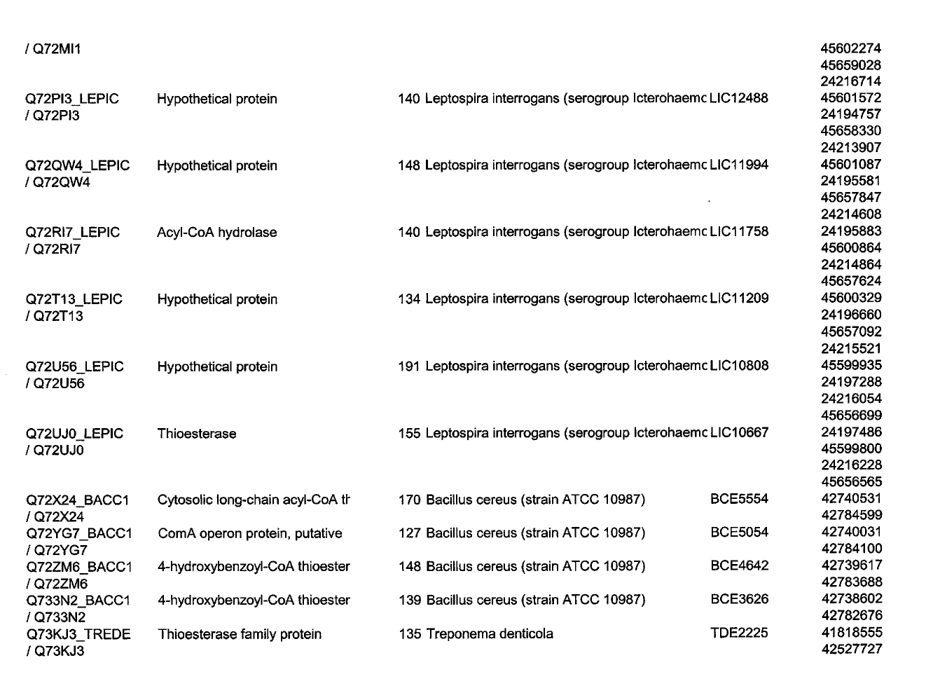

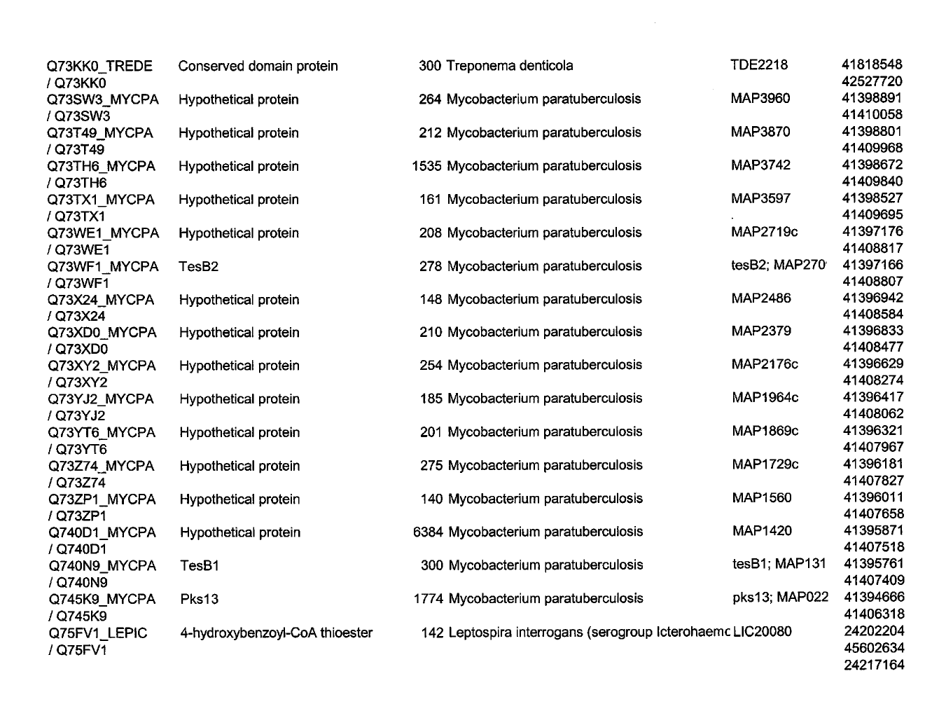

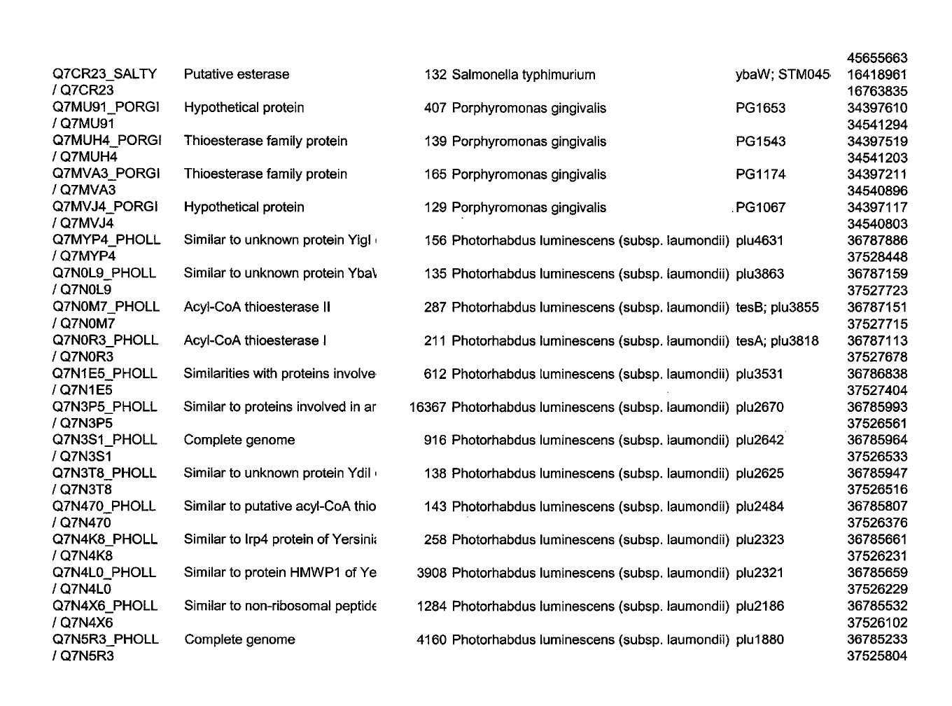

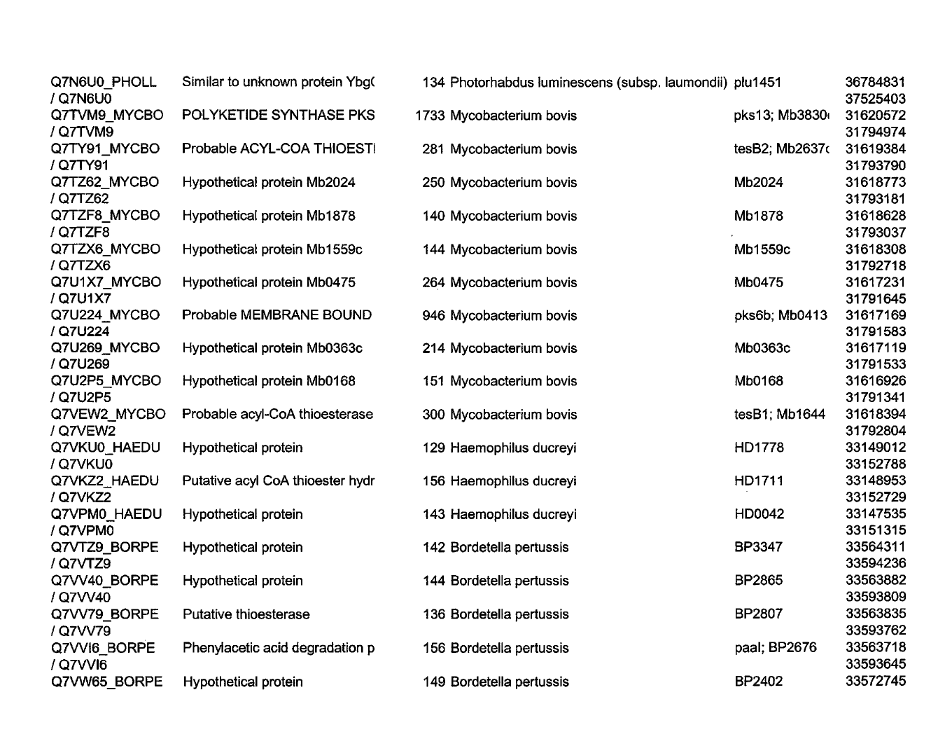

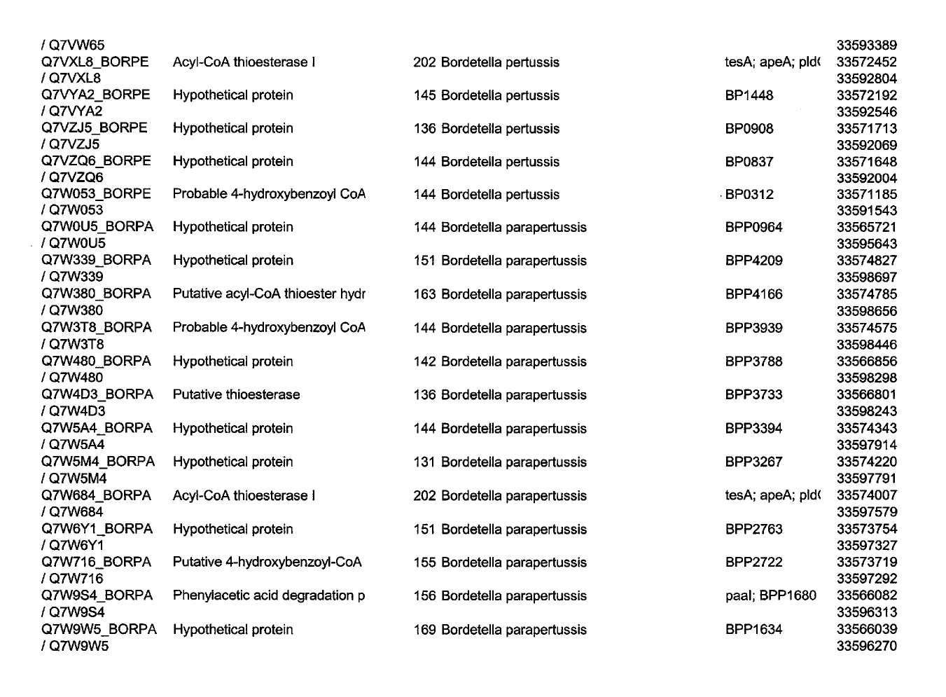

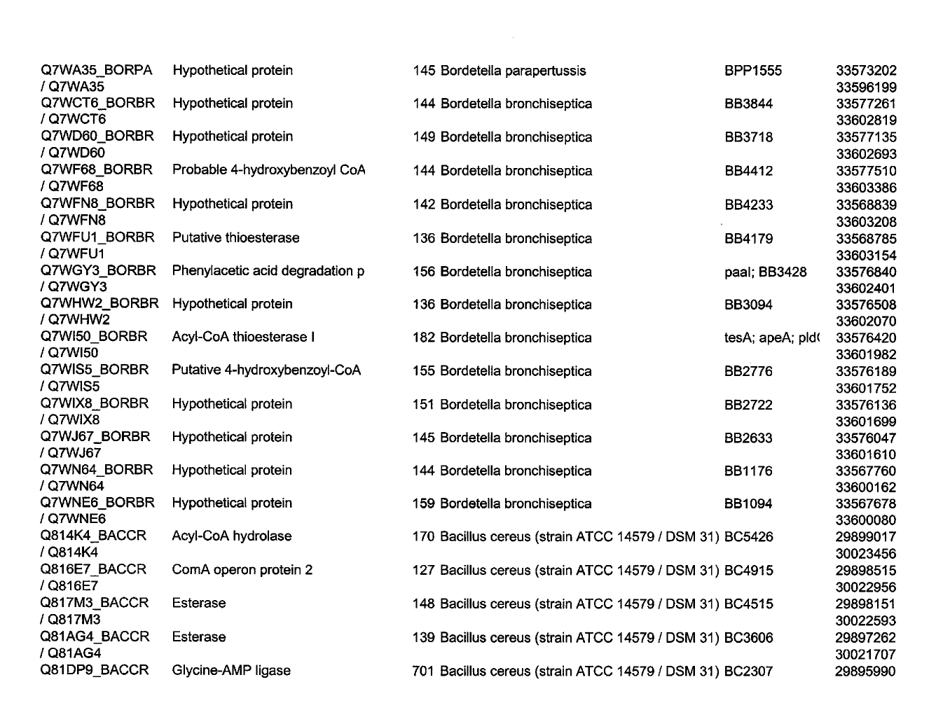

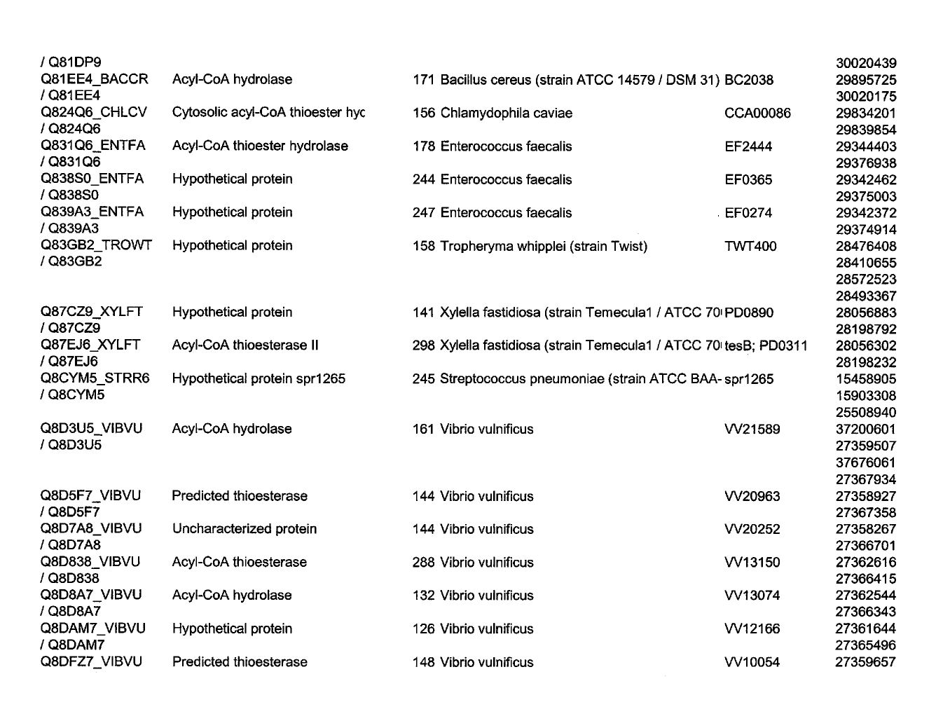

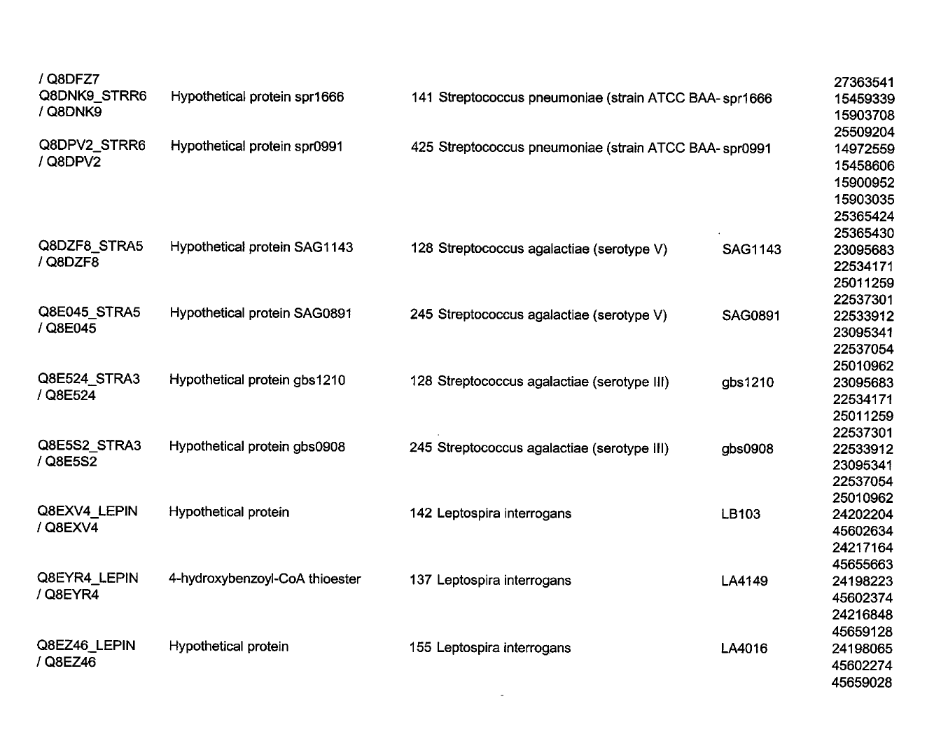

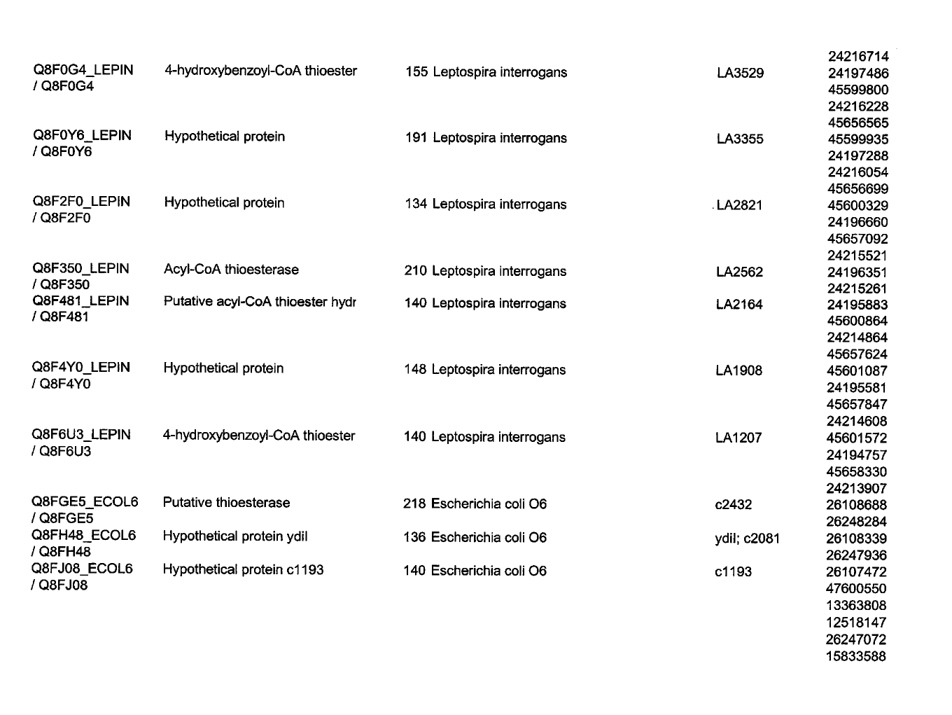

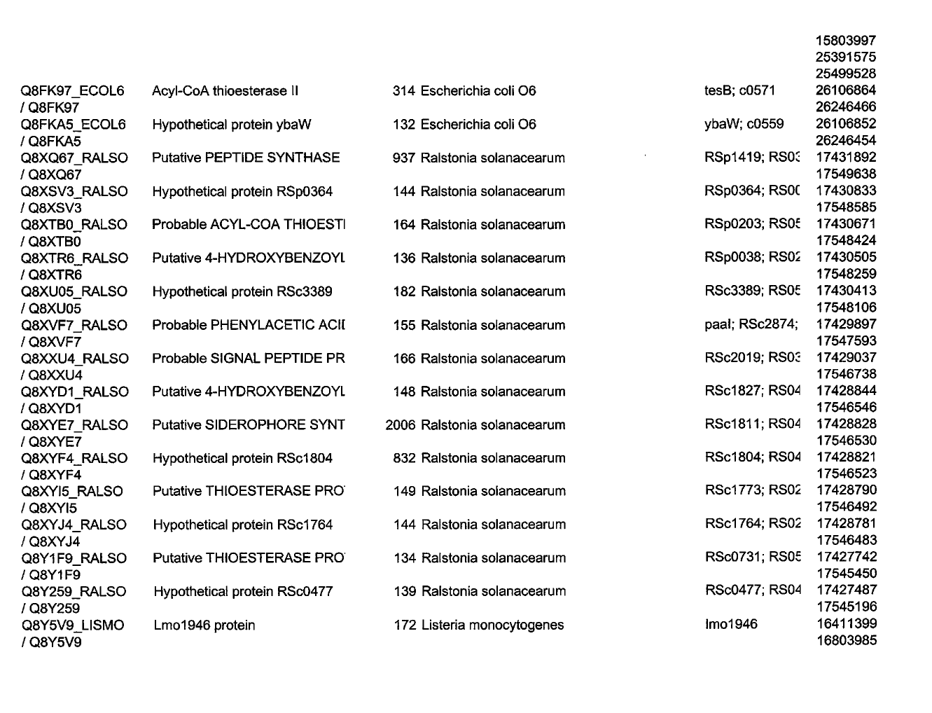

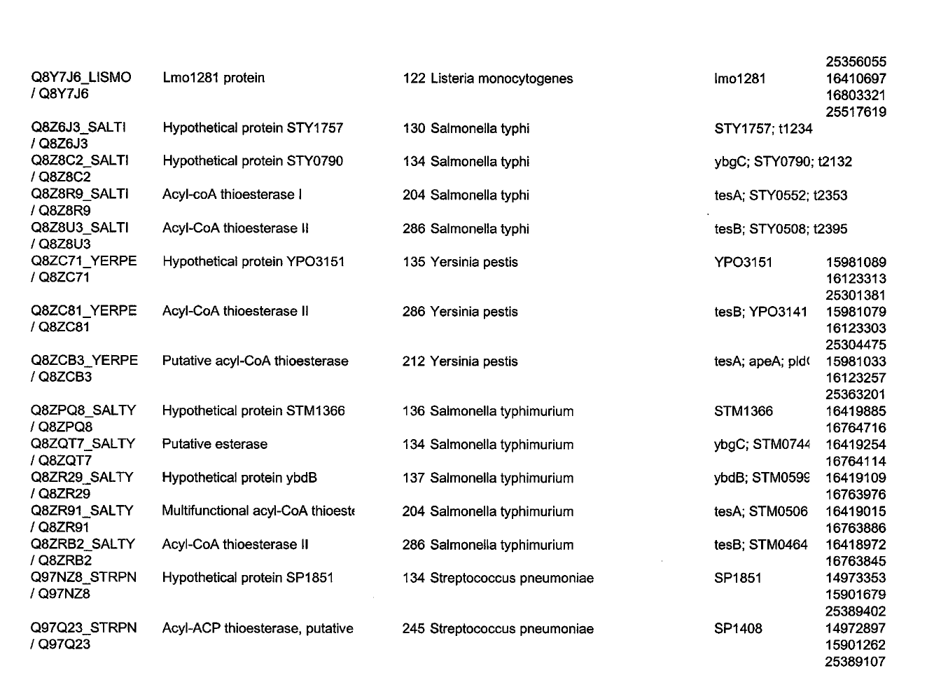

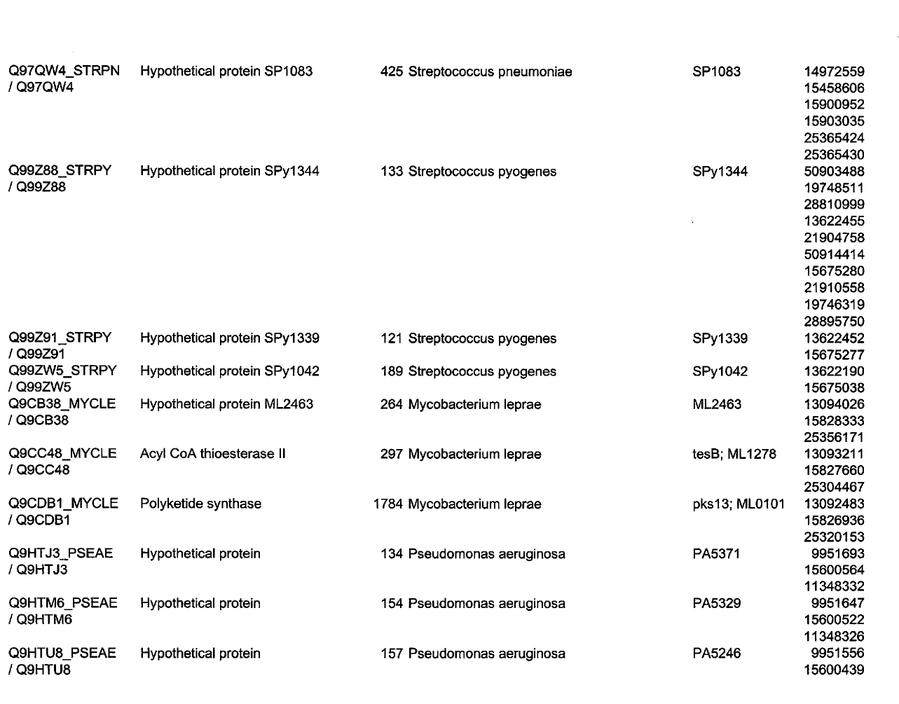

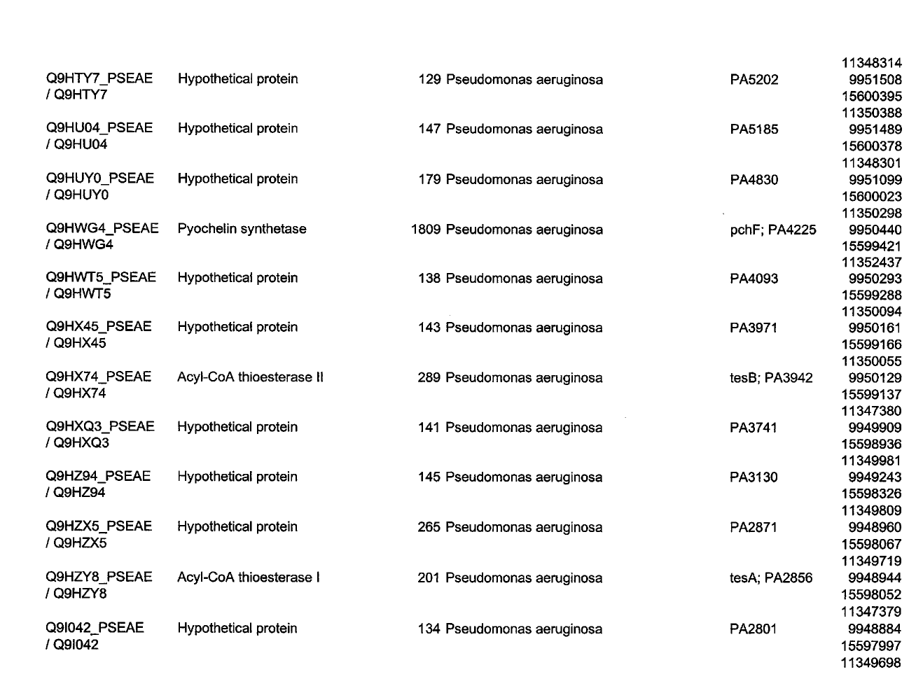

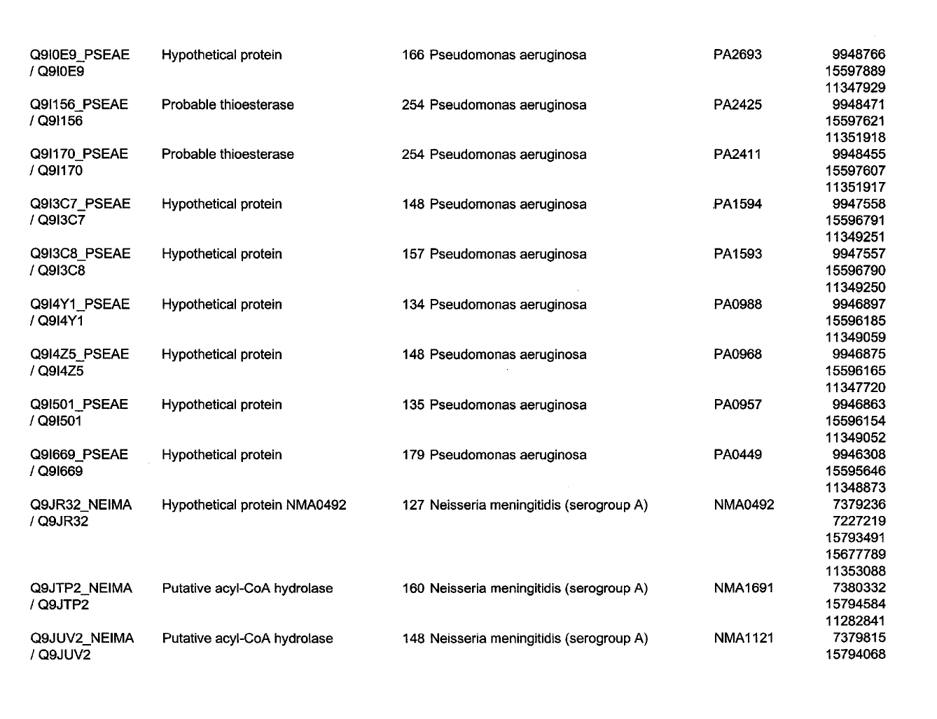

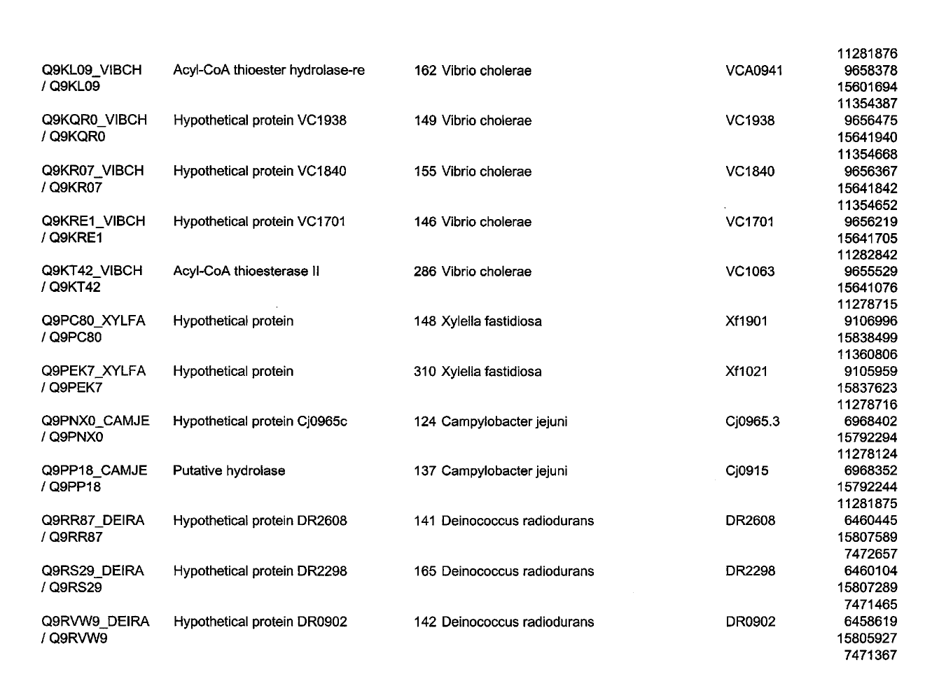

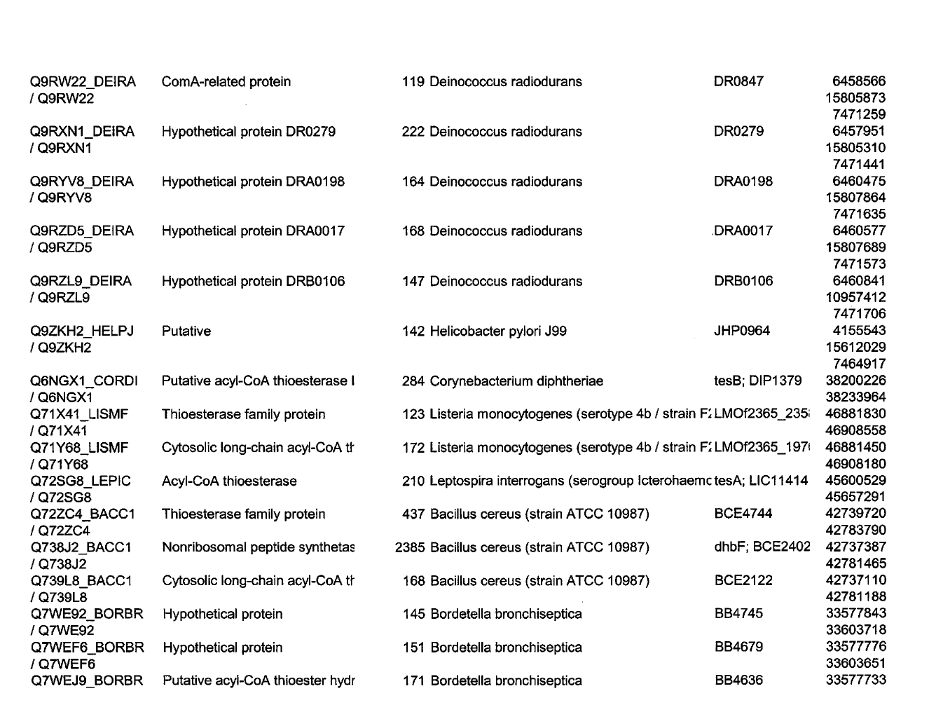

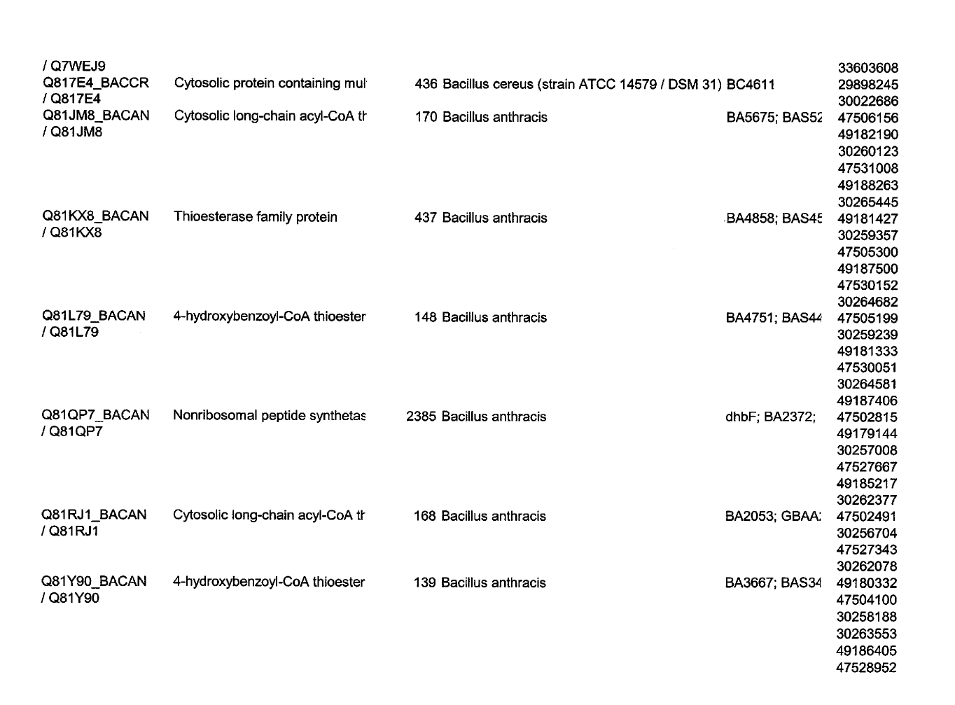

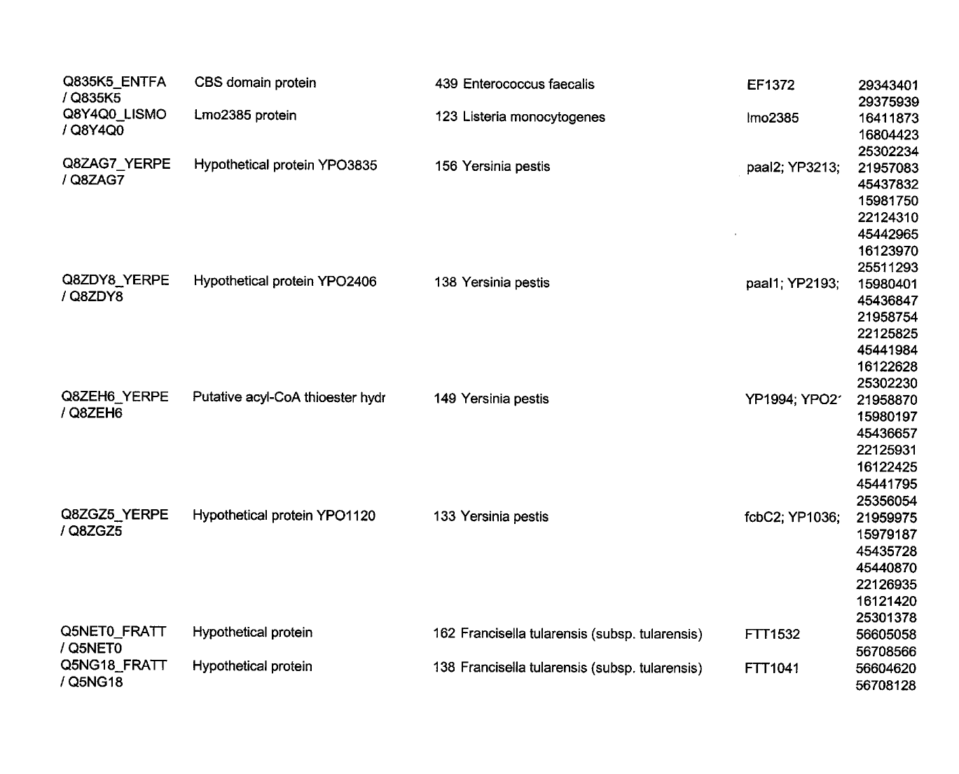

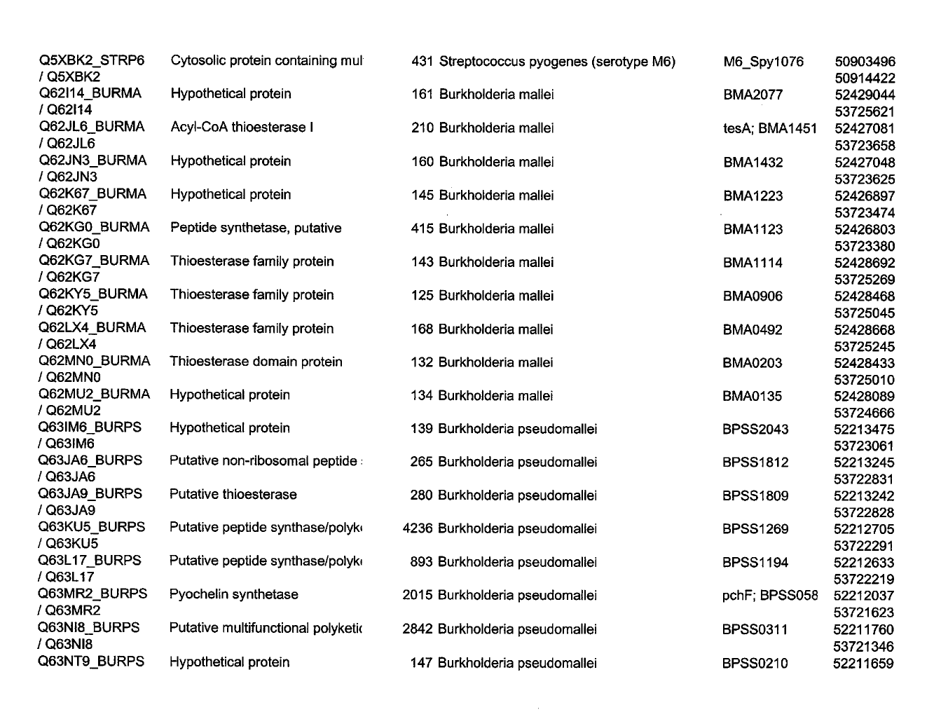

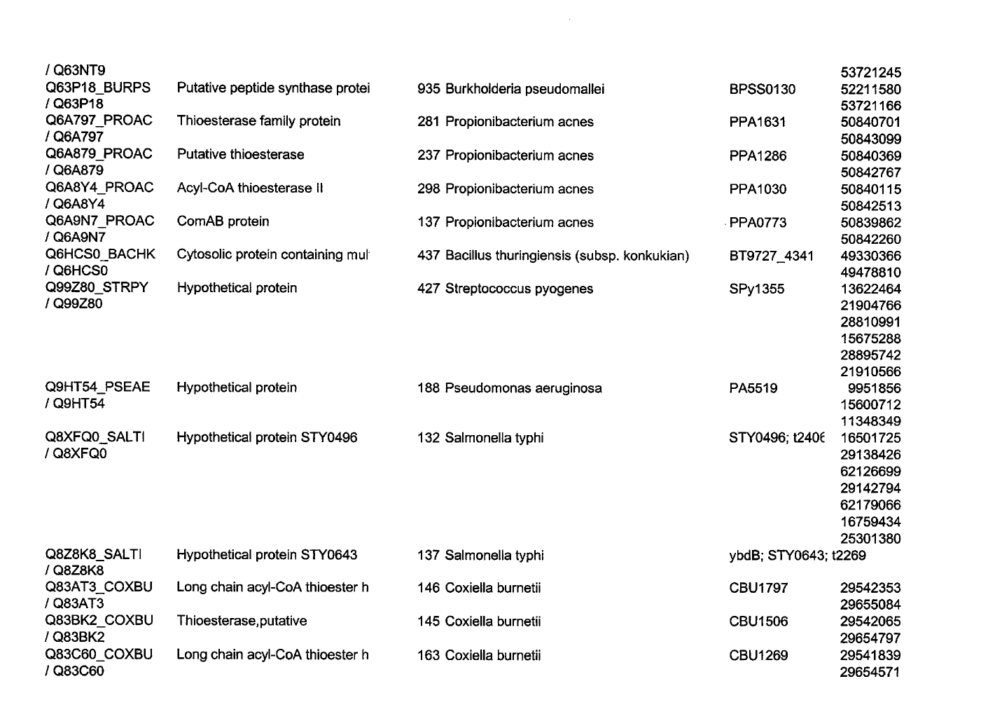

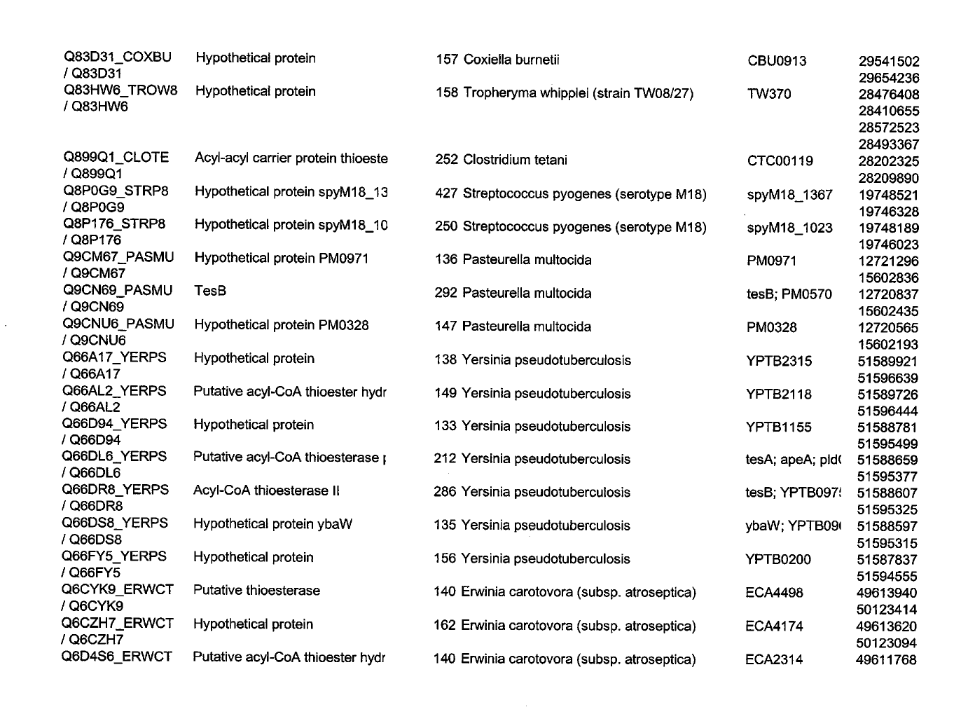

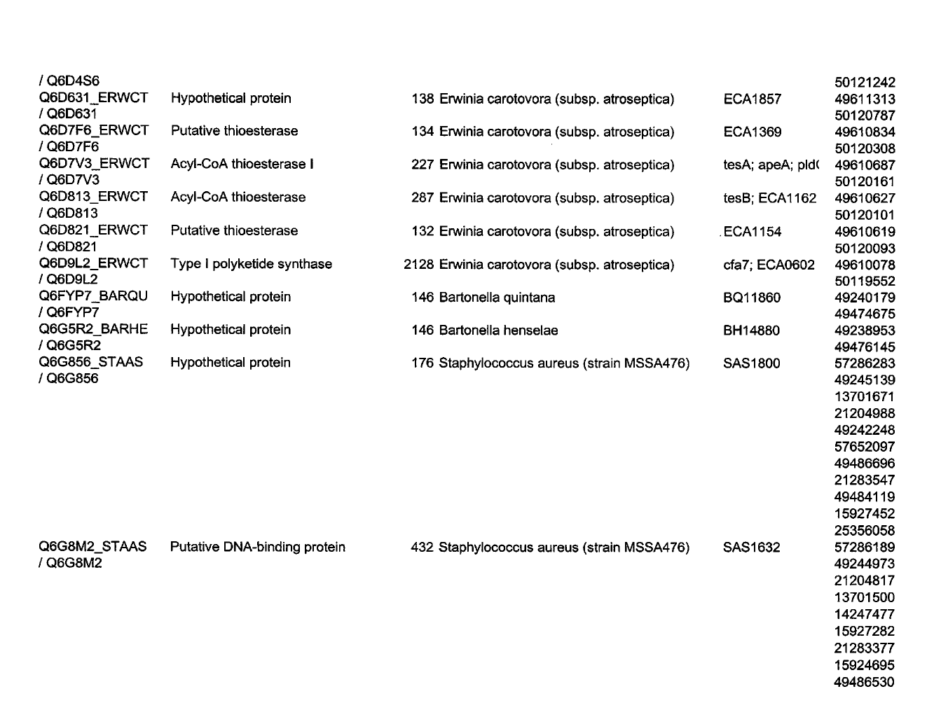

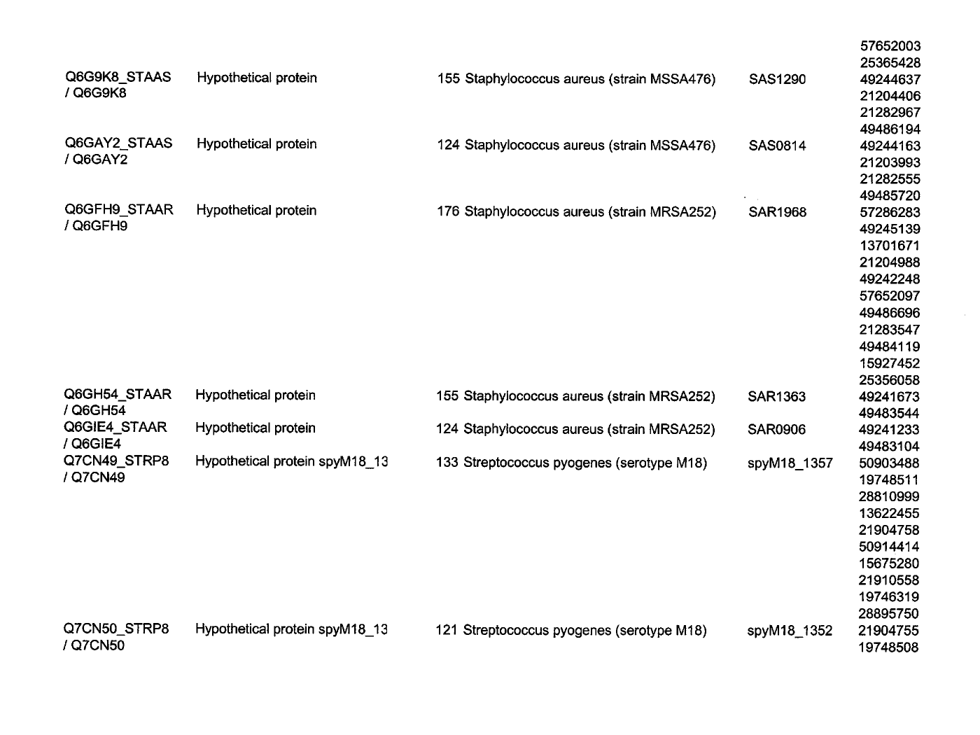

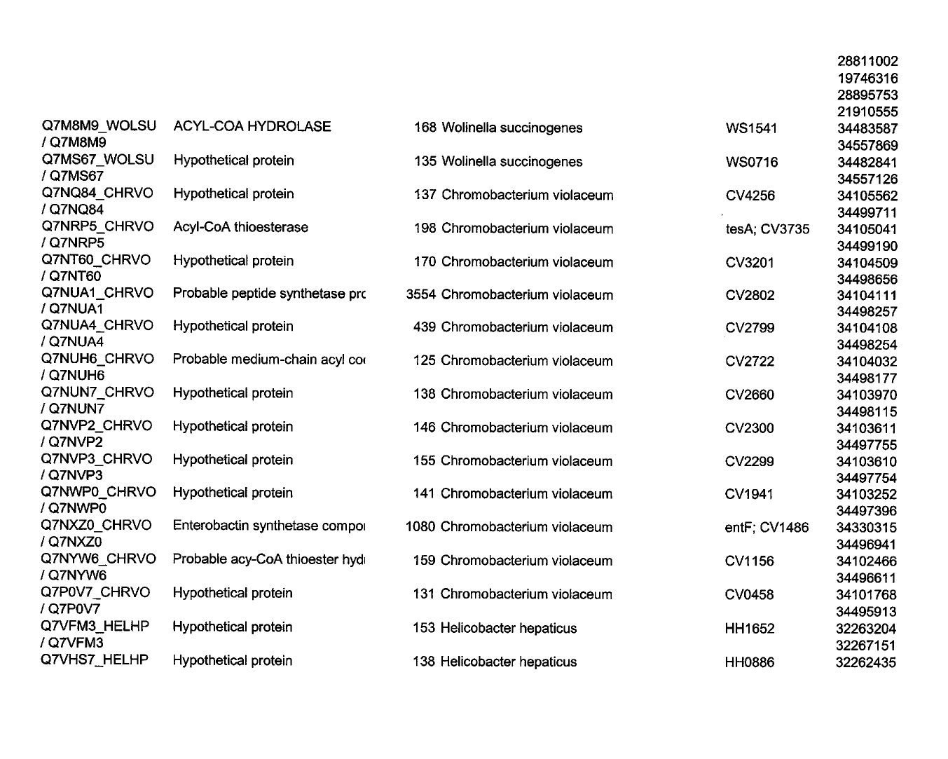

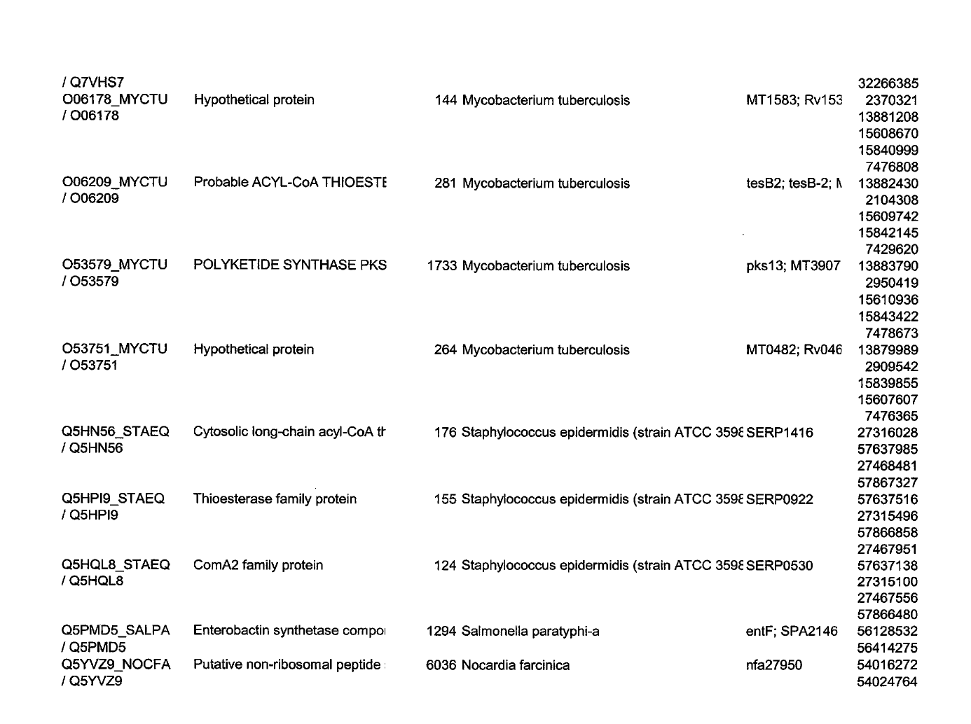

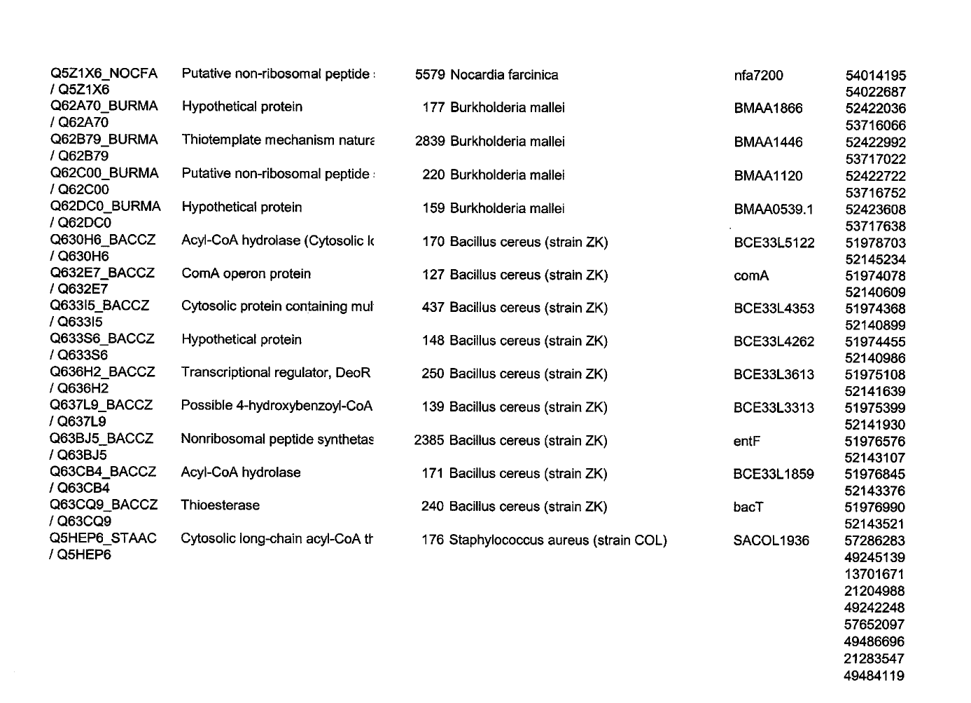

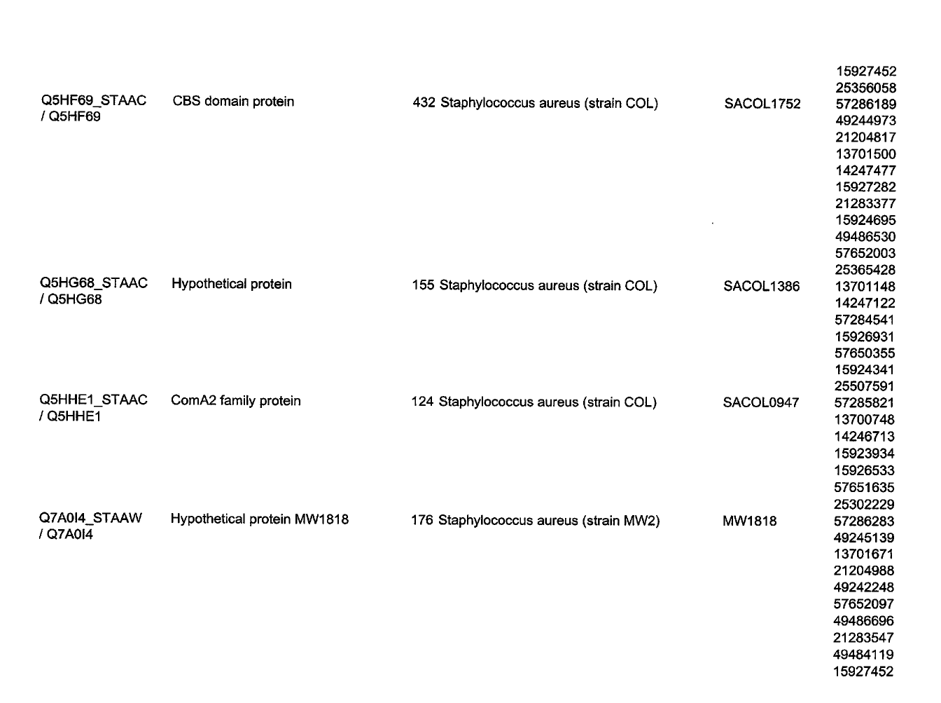

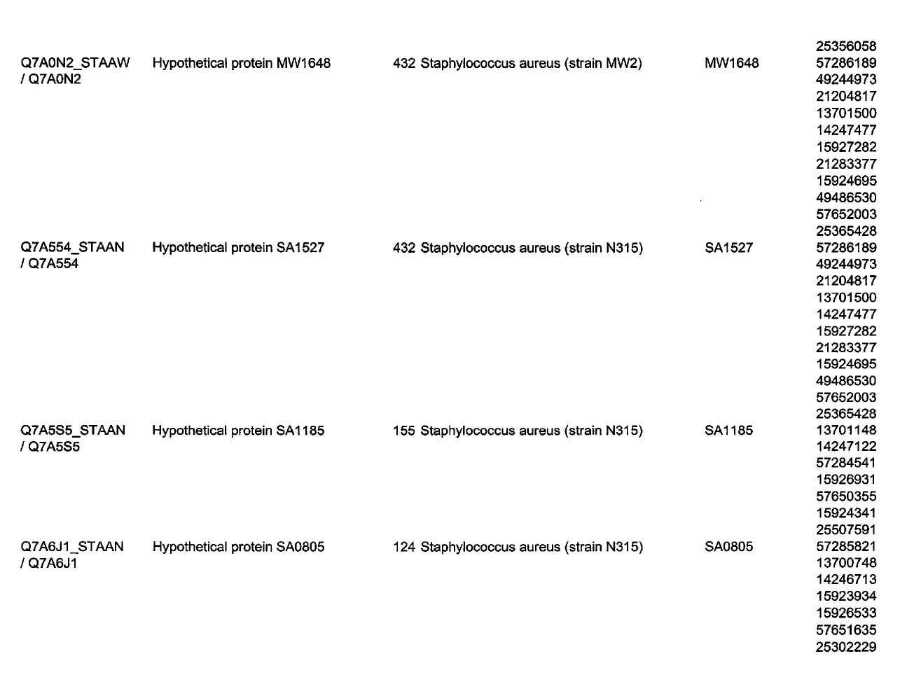

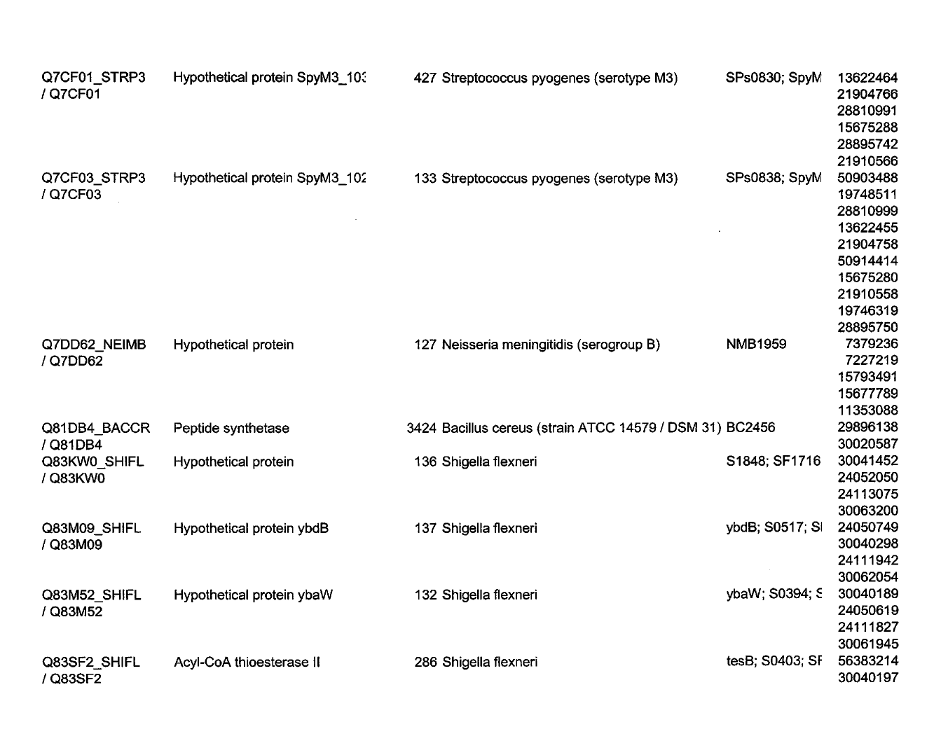

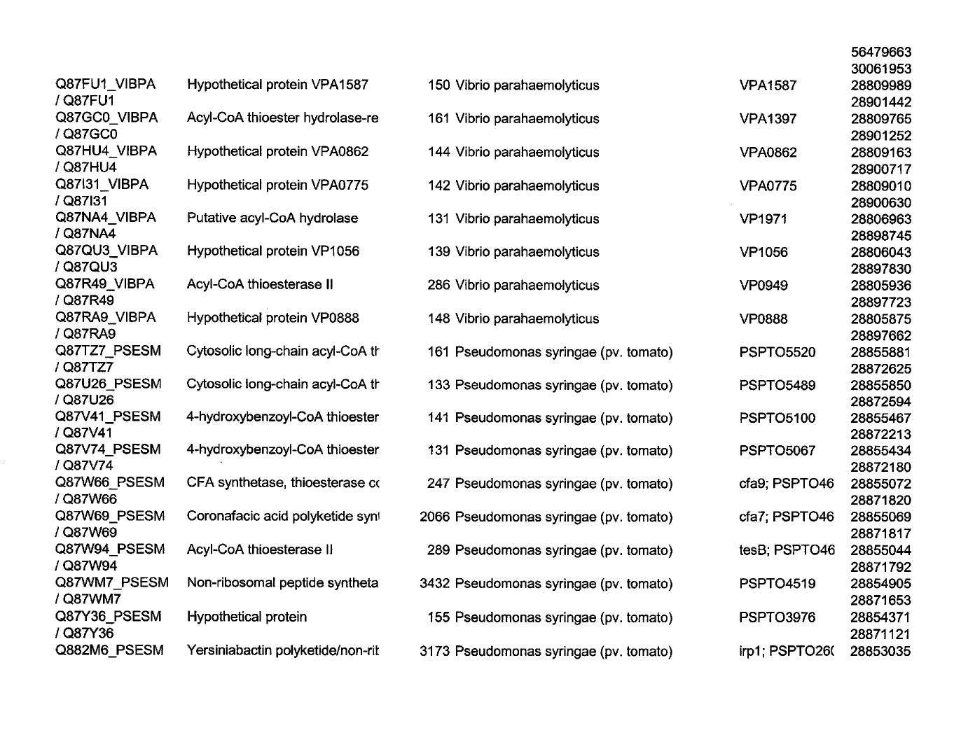

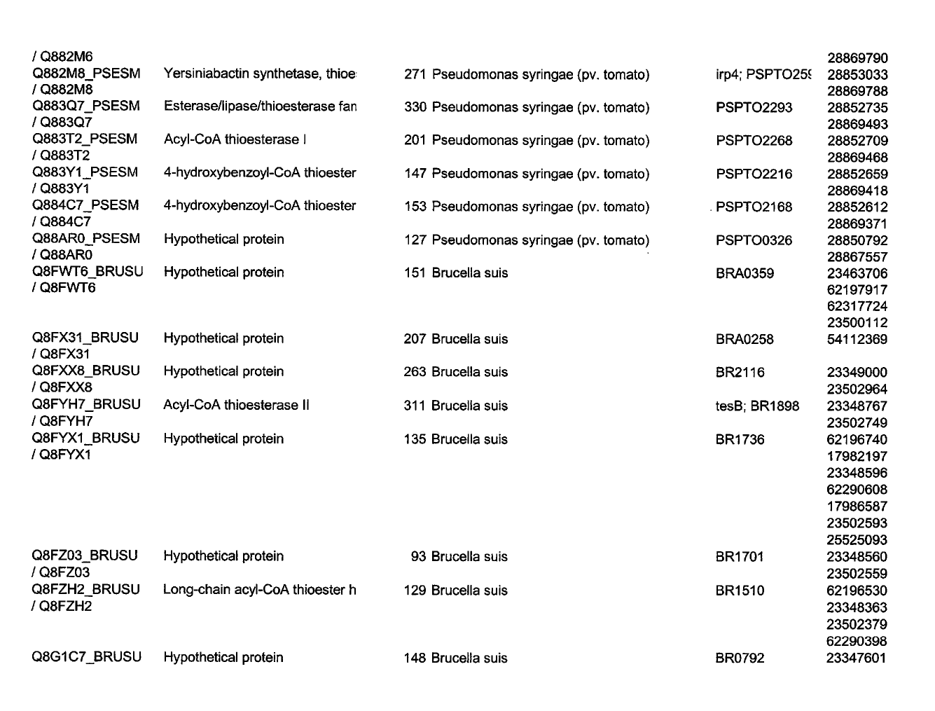

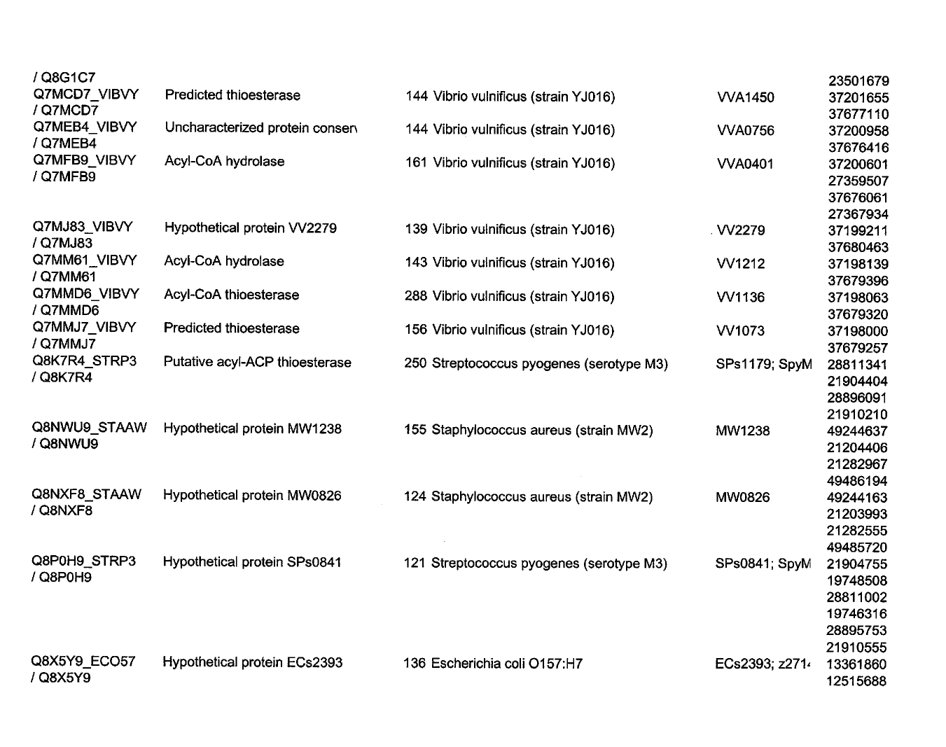

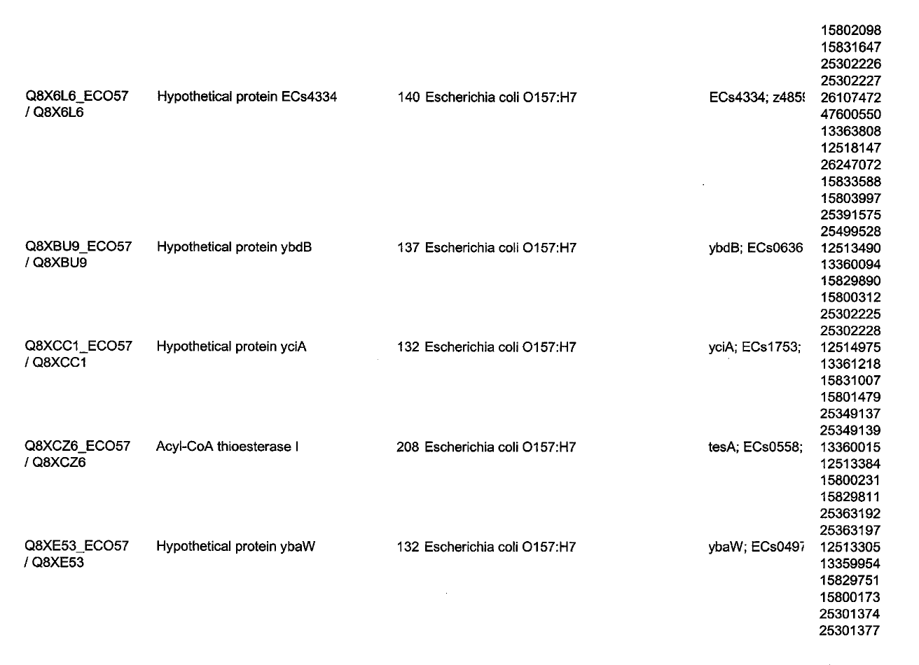

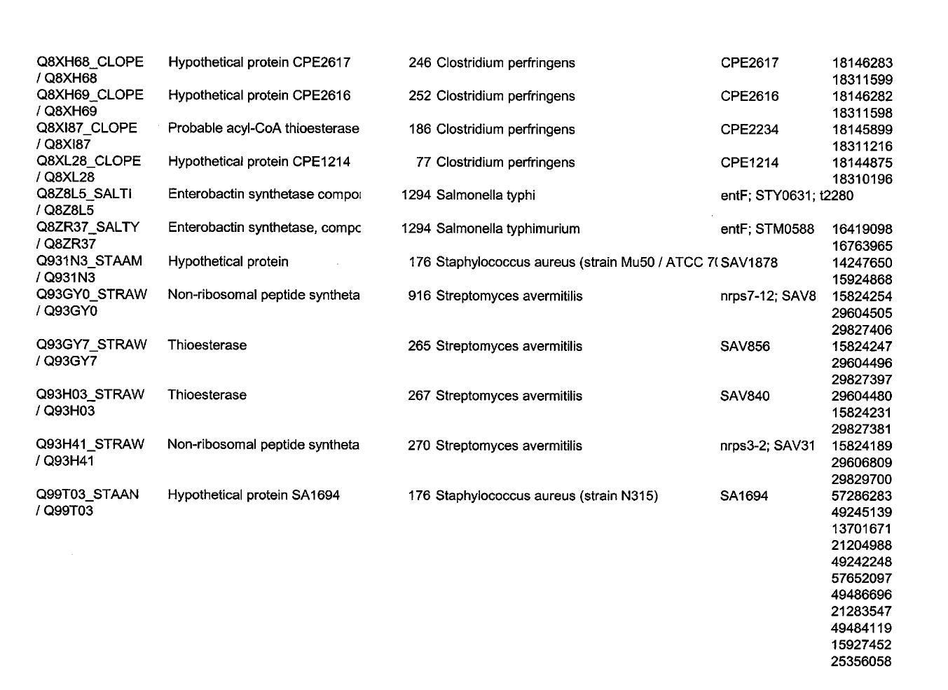

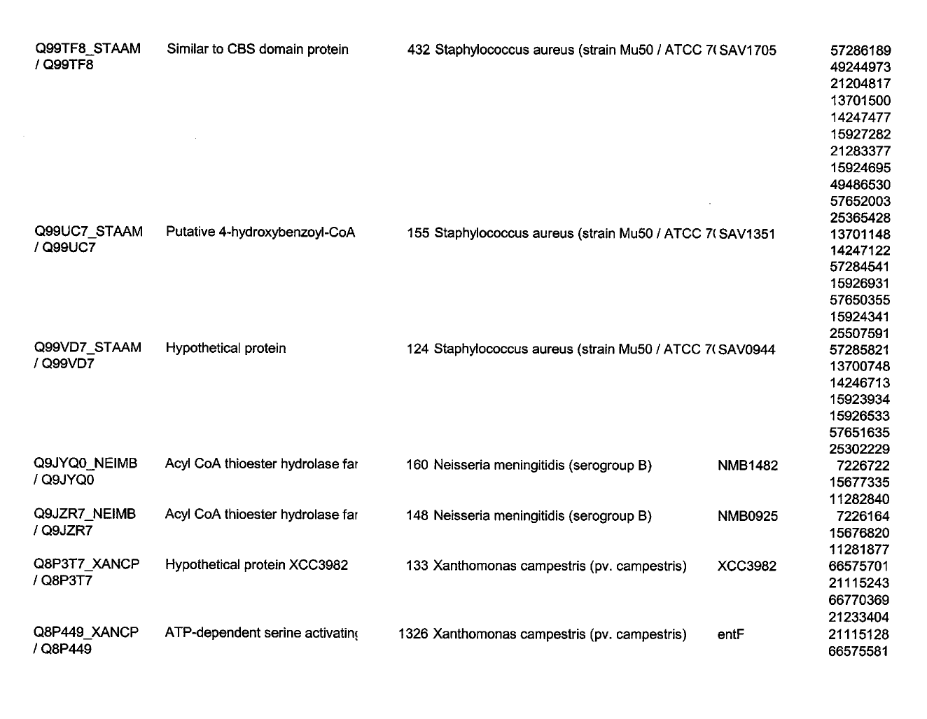

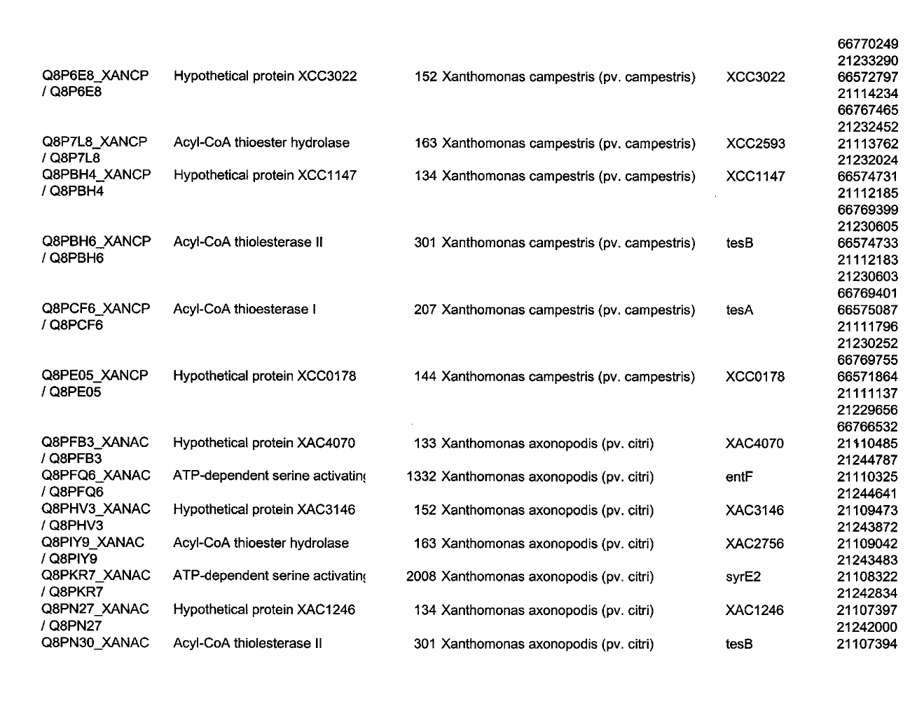

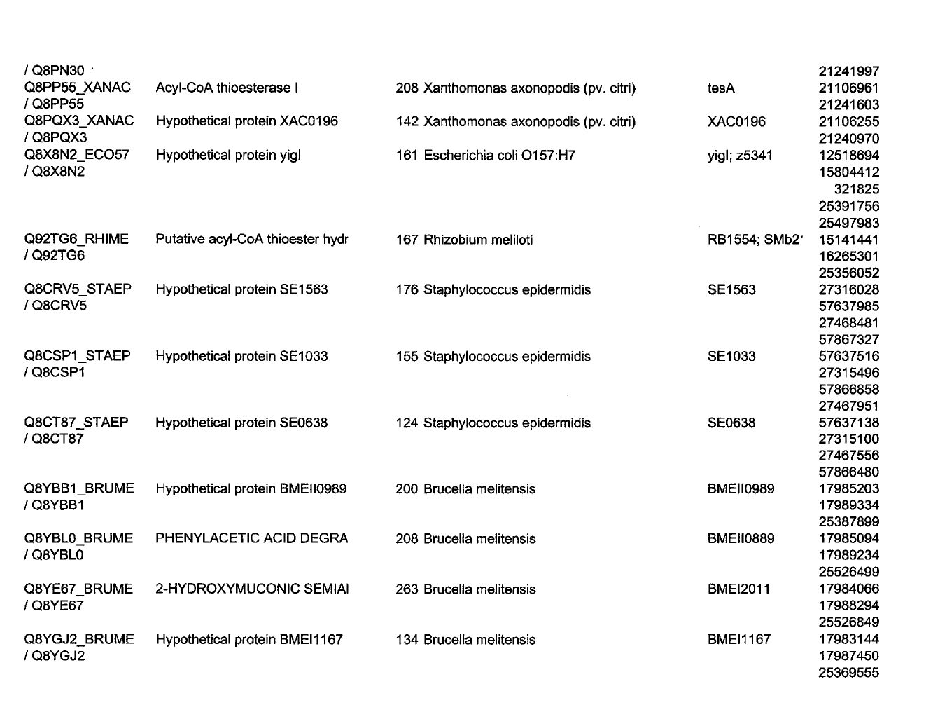

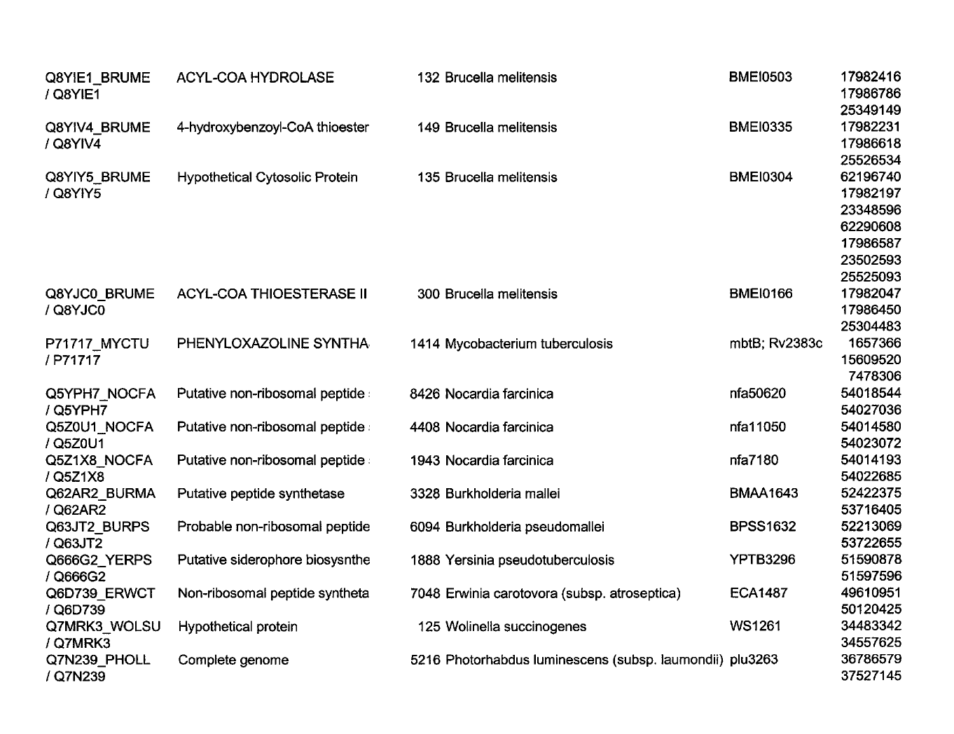

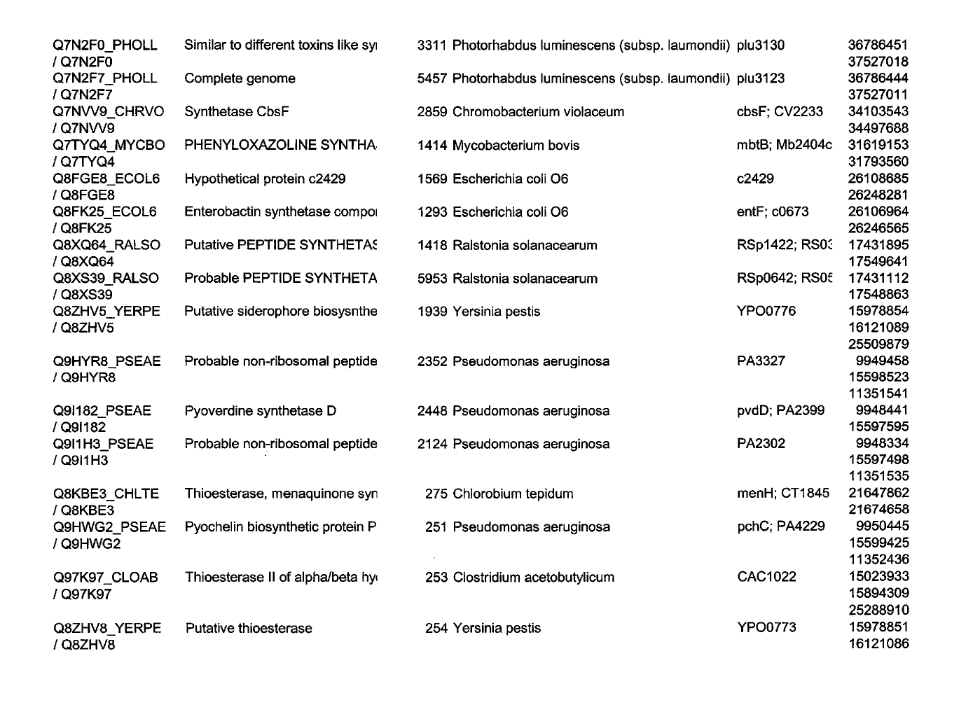

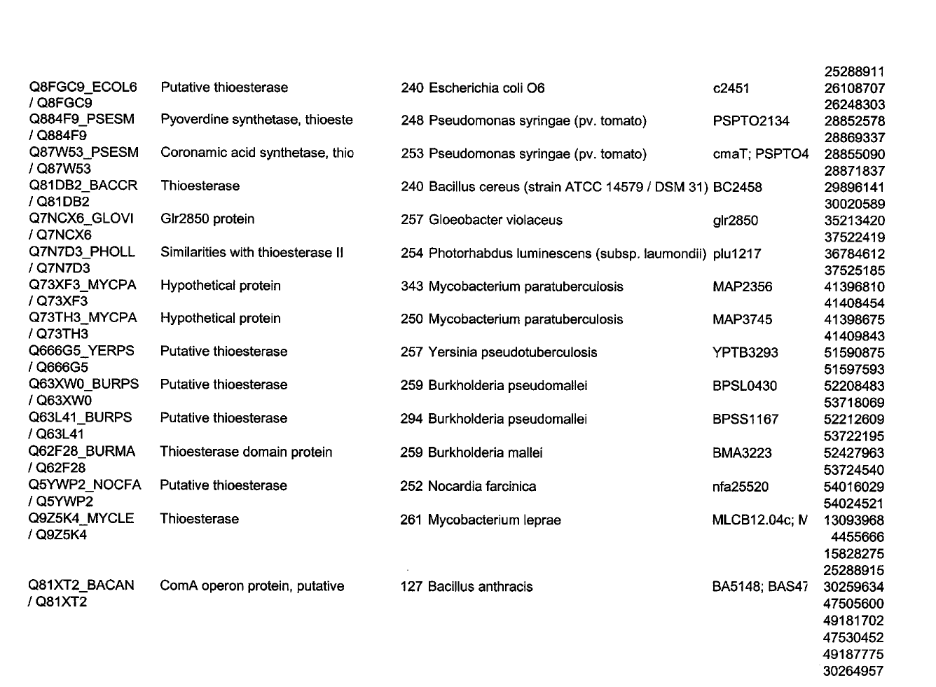

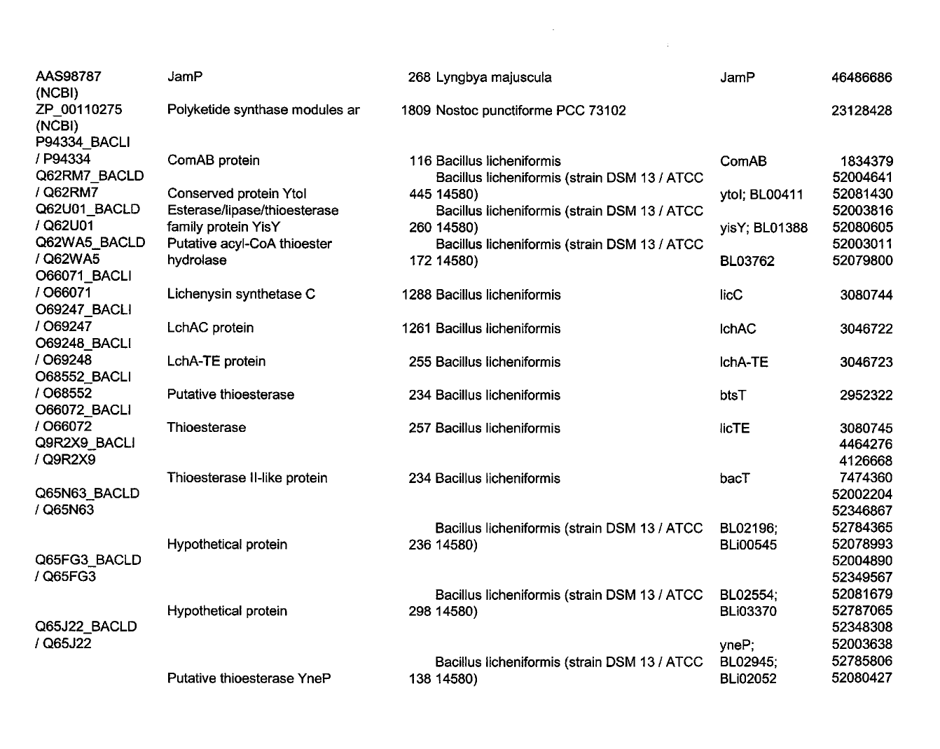

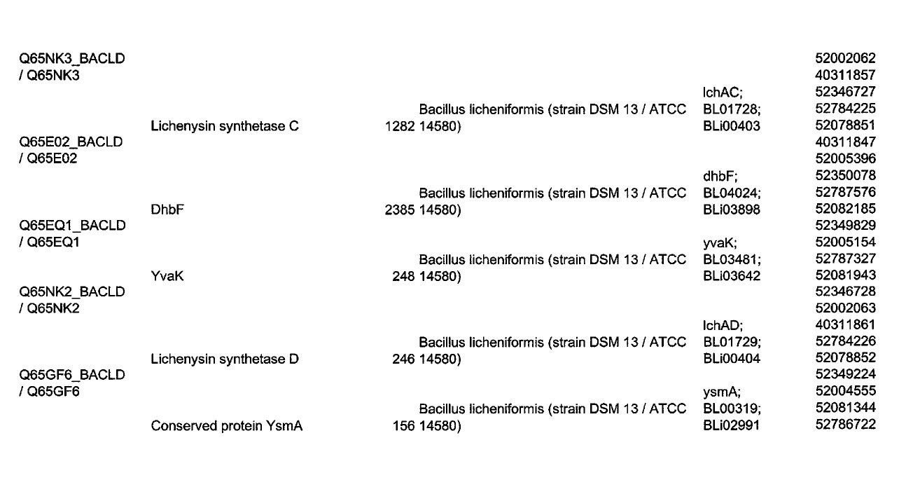

Identification of Antagonists of the FAS TE. The activity of the recombinant TE was assessed by its ability to cleave 4-methylumbelliferyl heptanoate (4-MUH), which is hydrolyzed to the fluorescent 4-methylumbelliferone (4-MU) (Jacks et al., Guilbault et al., 1969). To identify inhibitors of FAS TE, a library of 36,500 drug-like compounds was screened. The primary screen was conducted at a concentration of 12.5 μM of each compound, revealing 116 compounds that blocked >40% of the TE activity (FIG. 1). These compounds were retested to confirm activity, and a secondary screen was used to generate dose-response curves (data not shown). Eighteen compounds were identified with apparent Kᵢ<1.0 μM, eight of which contain a common barbituric acid pharmacophore. These barbituric acids, and derivatives thereof, were further studied. Comparative data for compounds in the presence of human FAS and Y. pestis YbtT are shown in FIGS. 5-6.

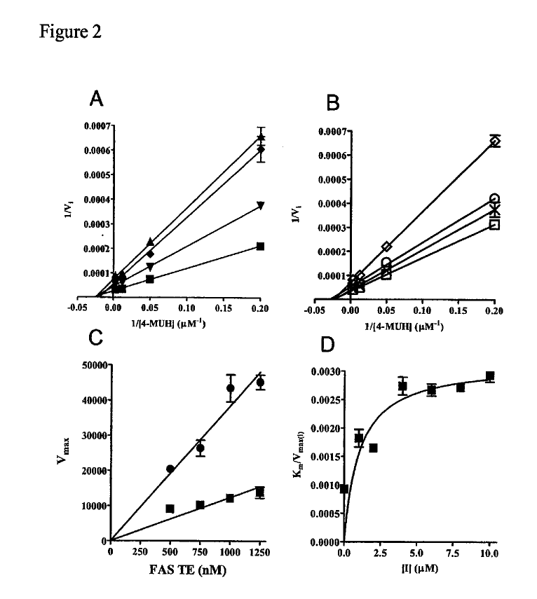

Barbituric Acid Derivatives Act as Partial Non-Competitive Inhibitors of FAS TE. Kinetic analysis was used to determine the Kᵢ for each compound, and to assess the general mechanism of their inhibition of the FAS TE (FIG. 2). Kinetic analysis was performed for compounds with high IC₅₀ values (5, 6, 11, 12), and are presented as representative plots. Double reciprocal plots reveal that compounds (1) and (7) are non-competitive inhibitors (FIGS. 2A and B) because the Kₘ for FAS TE for substrate is not influenced by the concentration of inhibitor. To confirm that the TE inhibition by the barbituric acid derivatives is non-competitive and reversible, Vₘₐₓ was measured as a function of the concentration of enzyme in the presence or absence of inhibitor (FIG. 2C). Since the slope of the inhibitor plot intersects the y-axis along with the uninhibited control, the Vₘₐₓ is unchanged in the presence of inhibitor as would be expected of a reversible inhibitor (Sigal, 1993). To distinguish partial versus pure non-competitive inhibition the Kₘ/Vₘₐₓ₍ᵢ₎ was plotted as a function of the concentration of inhibitor (FIG. 2D). A representative plot using compound (1) shows a hyperbolic curve as opposed to a linear plot. Hence, the compound is a partial non-competitive inhibitor; that is, it can bind to both the free enzyme and to the enzyme-substrate complex, and the enzyme-substrate-inhibitor (ESI) complex has reduced enzymatic activity.

Barbituric Acid Derivatives Inhibit the FAS Holoenzyme. As a first step toward testing the ability of the TE antagonists to inhibit FAS, their ability to block the site-specific labeling of the TE active site in the FAS holoenzyme was measured. This was accomplished by using FP-BODIPY, an activity-based probe containing a fluorophosphonate that reacts specifically and covalently with serine hydrolases. The fluorescent BODIPY reporter allows visualization of labeled enzymes on SDS-PAGE.

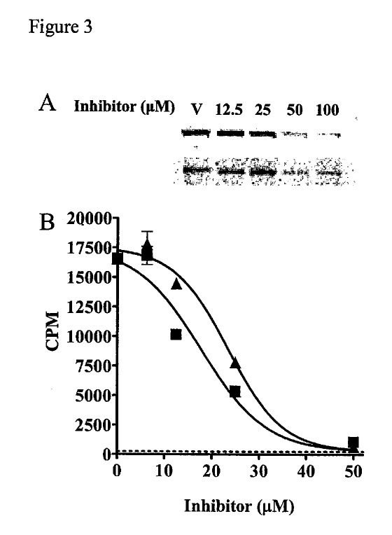

Hence, labeling of the holoenzyme can be tested by measuring competition between FP-BODIPY and potential antagonists. Compounds (2, 3) were used as exemplary antagonists in this assay. Both compounds inhibited binding of FP-BODIPY with complete inhibition occurring at approximately 50 μM (FIG. 3A). These observations show that the barbituric acid derivatives inhibit the TE within the context of the FAS holoenzyme. However, the IC₅₀ values are not accurate reflections of the Kᵢ of the compound because the activity-based probe irreversibly labels the enzyme in a covalent manner.

As a second step, the effect of the compounds or fatty acid synthesis in cell lysates, where the FAS holoenzyme remains active, was measured. The incorporation of [¹⁴C]-malonyl CoA, a precursor of palmitate, into fatty acids was measured according to methods described in Kuhajda et al. (1994). Treatment of cell lysates with compounds (1, 2) (6.3 to 50 μM) completely abrogated fatty acid biosynthesis in cell lysates (FIG. 3B). Half-maximal inhibition was observed at approximately 20 μM for each compound shown.

The Novel Barbituric Acid Derivatives are Cytotoxic to MDA-MB-435 Mammary Carcinoma Cells. Since other inhibitors of FAS elicit tumor cell death, the response of MDA-MB-435 cells to the barbituric acids was assessed by measuring cell viability 48 hours after treatment. Dose response curves were generated (data not shown) for representative compounds (1, 2, 7, 8) to calculate IC₅₀ values (Table 3). The IC₅₀ values for compounds (1, 2) are 20.64 and 14.21 μM, respectively. These values roughly correspond to the concentrations required for 50% inhibition of fatty acid biosynthesis (see FIG. 3B). This observation is generally consistent with the idea that the cytotoxic effects of the compounds are a result of the inhibition of FAS in whole cells, although the

possibility that the barbituric acid derivatives react with additional cellular targets cannot be excluded. The IC₅₀ of compounds (7, 8) for inhibition of fatty acid synthesis was not determined, but they elicited cytotoxicity at concentrations 1.6 and 9.5 μM, respectively, slightly lower than compounds (1, 2).

TABLE 3
Chemical structures and activities of inhibitors

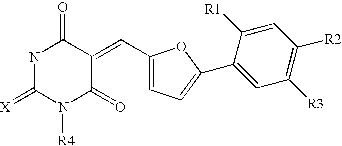

| | | | | | | | | Cytotoxicity |
|---|---|---|---|---|---|---|---|---|
| Name | X= | R₁= | R₂= | R₃= | R₄= | Kᵢ(μM) | ClogP | IC₅₀(μM) |
| RDR019(1) | S | Br | H | CH₃ | H | 0.11 | 3.998 | 20.64 |
| RDR102(2) | O | Br | NO₂ | H | CH₃ | 0.10 | 2.858 | 14.21 |
| | | | | | | | | Cytotoxicity |
| Name | X= | R₁= | R₂= | R₃= | R₄= | IC₅₀(μM) | ClogP | IC₅₀(μM) |
| RDR924(3) | S | NO₂ | OCH₃ | H | H | 4.4 | 2.898 | ND |
| RDR423(4) | S | H | CO₂ | H | H | 5.3 | 2.679 | ND |
| RDR256(5) | O | OH | H | NO₂ | H | 9.2 | 1.478 | ND |
| RDR317(6) | O | CO₂ | H | H | H | 29.0 | 1.009 | ND |

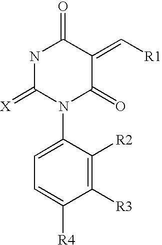

| | | | | | | | | Cytotoxicity |
|---|---|---|---|---|---|---|---|---|
| Name | X= | R₁= | R₂= | R₃= | R₄= | Kᵢ(μM) | ClogP | IC₅₀(μM) |
| RDR755(7) | O | 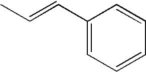 | OCH₃ | H | OCH₃ | 0.12 | 2.659 | 1.61 |
| | | | | | | | | Cytotoxicity |
| Name | X= | R₁= | R₂= | R₃= | R₄= | IC₅₀(μM) | ClogP | IC₅₀(μM) |
| RDR914(8) | O | 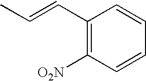 | H | H | H | 1.5 | 2.394 | 9.53 |
| RDR203(9) | O | 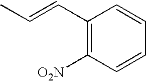 | H | OCH₃ | H | 2.0 | 2.313 | ND |
| RDR057(10) | S | 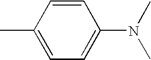 | F | H | H | 4.3 | 3.147 | ND |
| RDR506(11) | O | 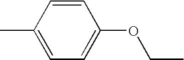 | H | H | OCH₂CH₃ | 14.5 | 2.943 | ND |
| RDR564(12) | O | 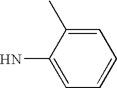 | H | CH₃ | H | 104.7 | 2.795 | ND |

FAS TE was pre-incubated with varied concentrations of test compounds or vehicle (DMSO) for 30 minutes at 37° C.
4-MUH was added (varied concentrations for Kᵢ calculations and 120 μM for IC₅₀ calculations).
Fluorescence was measured every five minutes for 40 to 60 minutes.
To measure cytotoxicity, MDA-MB-435 breast carcinoma cells were treated with varied concentrations of test compounds and incubated for 48 hours.
Media was aspirated and replaced with fresh media containing MTS and PMS.
Plates were further incubated for 2 hours and read at 490 nm.
ND = not determined.

Discussion

The objective of the study was to identify novel antagonists of the TE of human FAS. With this objective, more than 35,000 drug-like compounds were screened and two structurally distinct classes of barbituric acids that are potent antagonists of the FAS TE were identified. These compounds: 1) act as reversible non-competitive inhibitors of the recombinant TE, 2) inhibit the TE on the FAS holoenzyme and block fatty acid synthesis, and 3) elicit tumor cell death. Based on these observations, barbituric acid derivatives represent a unique class of FAS antagonists that may be useful as antineoplastic agents.

The barbituric acid derivatives described here fulfill the Lipinski rule-of-five analysis, a guideline used by the pharmaceutical industry to identify drug-like molecules for pre-clinical development (Lipinski et al., 1997). In particular, compounds (1-12) exhibit calculated log P (C log P) values of less than 4 (see Table 3), a measurement indicating low hydrophobicity. Lead compounds of C log P>5 are less likely to be successful drug candidates due to poor absorption and membrane permeability. The FAS inhibitor orlistat for example, is highly insoluble under physiological conditions (C log P=8.609), with current use limited to the gut. For this reason, barbituric acid derivatives likely represent an acceptable pharmacophore for development of drugs targeting FAS.

The screen for FAS TE antagonists was performed using the non-natural substrate 4-methylumbelliferyl heptanoate as a mimic of the natural substrate. While the inhibitors may behave differently with the natural substrate palmitate, the results argue against this possibility. First, the barbituric acids inhibit the active site of the TE in the context of the FAS holoenzyme, and also block fatty acid synthesis by the enzyme. Therefore, the simplest interpretation of the findings is that the 4-MUH substrate is a reasonable mimic of the natural substrate and that the identified barbituric acids can antagonize the TE in near physiologic conditions.

The findings also show that the barbituric acid derivatives are non-competitive antagonists of the TE, meaning that they bind to both unoccupied enzyme and to the enzyme-substrate complex, and that they act by reducing the turnover of substrate. This property may offer important advantages in drug development, especially in developing antagonists of FAS. FAS is a multi-domain enzyme, and contains an ACP to which the evolving alky chain of the fatty acid is bound during biosynthesis. The resulting palmitoyl-ACP is just 48 Å from the TE active site (Yuan et al., 1986) where it is hydrolyzed to free palmitate. Hence, the effective concentration of substrate for the TE is high and traditional competitive inhibitors must meet a high hurdle in order to compete with endogenous substrate. The fact that the barbituric acid inhibitors of the TE are non-competitive may overcome this issue because they do not act by competing with substrate.

Recent work has raised the awareness that some classes of compounds act as promiscuous non-competitive inhibitors by causing protein aggregation (Feng et al., 2005). This possibility can be excluded from the current set of FAS antagonists for the following reasons. First, the same barbituric acids identified here were tested against other structurally homologous TEs, like the ybtT and the HMWP-1 thioesterases from Yersinia pestis (Miller et al., 2002) (FIGS. 5-6). The barbituric acids reported here failed to inhibit these TEs in the concentration range in which they were effective for FAS. This observation is inconsistent with what one would expect of a “promiscuous” aggregator as described by Feng et al. (2005). Furthermore, the activity-based probe FP-BODIPY was used to gauge the effect of the barbituric acids on many other serine hydrolases in lysates of MB-MDA-435 cells, and most were found to be unaffected at concentrations of the barbituric acid of up to 100 μM (data not shown). This observation is also inconsistent with the expected behavior of a compound that causes promiscuous protein aggregation.

The core barbituric acid moiety found in the TE inhibitors is common to drugs like phenobarbital and pentobarbital. Given the similarity in chemical structure between these drugs and the TE antagonists, it was important to assess their ability to inhibit the FAS TE. Phenobarbital and the core barbiturate moiety were tested for the ability to inhibit the FAS TE and both were found to be without effect at concentrations up to 100 μM (data not shown). Additionally, the FAS TE lacks any structural homology to the GABA-mediated chloride channel family of proteins targeted by phenobarbital and pentobarbital (MacDonald et al., 1989; Olsen et al., 1982; Richards et al., 1976). Modeling of pentobarbital binding illustrates steric hindrance of 5′-methylbutyl side chains with amino acids protruding from the ion channel (Arias et al., 2001; Dodson et al., 1990; Arias, 1998). Bulky ring structures at positions 1 and/or 5 on the pyrimidine ring found in the TE inhibitors may likewise inhibit physiologic binding to targets of current clinical barbiturates.

Thus, the barbituric acid derivatives described herein block fatty acid synthesis, exhibit cytotoxicity in breast cancer cells, and satisfy the Lipinski rule-of-five analysis. Interestingly, it appears that there has been no report of a connection between the barbituric acid pharmacore and FAS or other serine hydrolases.

### EXAMPLE II

FIGS. 5-6 show Kᵢ and percent inhibition data for human FAS TE and Yersinia ybtT for 46 and 83 compounds, respectively. Compounds that inhibit human FAS TE at least about 2-fold better than Yersinia ybtT are compounds U.S. Pat. Nos. 5,215,341, 5,992,802, 6,237,848, 6,238,046, 5,621,839, 5,627,858, 6,237,946, 6,222,372, 5,550,263, 6,200,627, 6,238,569, 5,399,387, 5,155,680, 5,155,679, 5,670,760, 5,809,324, 5,760,449, 5,869,438, 6,368,521, 5,630,339, 6,238,755, 5,843,019, 5,988,102, 6,238,616 and 5,810,505 (FIG. 5).

Compounds that inhibit Yersinia ybtT at least about 2-fold better than human FAS TE are compounds U.S. Pat. Nos. 6,108,152, 6,240,372, 6,137,752, 6,020,642, 5,555,858, 6,005,009, 6,013,885, 6,223,369, 6,232,755, 6,192,873, 5,579,479, 6,224,794, 5,604,372, 5,729,598, 5,865,028, 5,228,235, 5,228,252, 6,192,873, 5,228,245, 5,469,312, 5,471,481, 5,565,071, 5,622,028, 5,723,048, 5,990,503, 5,992,599, 5,839,928, 5,366,282, 5,376,366, 5,565,071, 5,767,664, 5,756,068, 5,808,414, 5,376,842, 5,539,742, 5,769,209, 5,584,572, 5,673,176, 5,735,629, 5,930,764, 5,987,008, 6,076,470, 6,191,930, 6,241,087, 6,103,437, 6,108,460, 5,628,173, 5,581,710, 5,180,296, 5,186,836, 5,626,567, 5,629,954, 5,739,333, 5,152,592, 5,185,714, 5,554,103, 5,572,814, 5,671,264 and 5,617,138 (FIGS. 5-6).

### REFERENCES

Adams et al., Biochem. J., 360:135 (2001).

Advanced Organic Chemistry Part B: Reactions and Synthesis, Second Edition, Cary and Sundberg (1983).

Advanced Organic Chemistry Third Edition, John Wiley & Sons, New York (1985).

Advanced Organic Chemistry Reactions Mechanisms, and Structure, Second Edition, March (1977).

Alo et al., Cancer, 77:474 (1996).

Arias et al., Mol. Pharmacol., 60:497 (2001).

Arias, Biochim. Biophys. Acta, 1376:173 (1998).

Bandyopadhyay et al., Oncogene (2005).

Banks et al., J. Infect. Dis., 190:727 (2004).

Beres et al., Proc. Natl. Acad. Sci. USA, 99:10078 (2002).

Biological Approaches to the Controlled Delivery of Drugs, Annals of the New York Academy of Sciences, Vol. 507, R. L. Juliano (editor) (1988).

Bobrov et al., Infect. Imm., 70:4204 (2002).

Cendrowski et al., Mol. Microbiol., 51:407 (2004).

Cheng et al., Biochem. Pharmacol., 22:3099 (1973).

Chou et al., Adv. Enzyme Regul., 22:27 (1984).

Cole et al., Nature, 393:537 (1995).

Comprehensive Organic Synthesis. Selectivity, Strategy & Efficiency in Modern Organic Chemistry. In 9 Volumes, Barry M. Trost, Editor-in-Chief (Pergamon Press, New York, 1993 printing).

Comprehensive Organic Transformations, Larock, R. C., Second Edition, John Wiley & Sons, New York (1999).

Compendium of Organic Synthetic Methods, John Wiley & Sons, New York, Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6, Michael B. Smith.

Conversion of Non-Toxic Pro-drugs to Active, Anti-Neoplastic Drugs Selectively in Breast Cancer Metastases, Basse, (2000).

Cunningham et al., Clin. Microbiol. Dev., 13:470 (2000).

Design of Biobiological Agent Properties through Pro-drugs and Analogs, Edward B. Roche (editor), Amer. Biological Agent Assn. (MacK) (1977).

Design of Pro-drugs, Hans Bundgaard (editor), Elsevier Science (1986).

Dodson et al., Br. J. Pharmacol., 101:710 (1990).

Enzyme Kinetics: Behavior and Analysis of Rapid Equilibrium and Steady-State Enzyme Systems, John Wiley & Sons, Inc. (1993).

Enzyme-Pro-drug Strategies for Cancer Therapy, Roger G. Melton (editor), Richard J. Knox (editor), Plenum Press (1999).

Feng et al., Nat. Chem. Biol., 1:146 (2005).

Ferreti et al., Proc. Natl. Acad. Sci. USA, 98:4658 (2001).

Gansler et al., Hum. Pathol., 28:686 (1997).

Geoffrey et al., Infect. Imm., 68:4452 (2000).

Guilbault et al., (1969) Anal. Chem., 41:2006 (1969).

Hadvary et al., J. Biol. Chem., 266:2021 (1991).

Handbook of Pharmaceutical Excipients (1986).

Huffman et al., J. Org. Chem., 60:1590 (1995).

Hydrolysis in Drug and Pro-drug Metabolism: Chemistry, Biochemistry, and Enzymology, Bernard Testa and Joachim Mayer; Vch Verlagsgesellschaft Mbh (2003).

Jacks et al., Anal. Biochem., 21:279 (1967).

Jernigan et al., Emerg. Inf. Dis., 7:933 (2001).

Kinsella et al., Proc. Natl. Acad. Sci. USA, 100:10320 (2003).

Knowles et al., J. Biol. Chem., 279:30540 (2004).

Kolattukudy et al., Mol. Microbiol. 24:263 (1997).

Kridel et al., Cancer Res., 64:2070 (2004).

Kuhajda et al., Proc. Natl. Acad. Sci. USA, 91:6379 (1994).

Leferre et al., Am. J. Hum. Genet., 69:1002 (2001).

Lipinski et al., Adv. Drug Del. Rev., 23:3 (1997).

Liu et al., Proc. Natl. Acad. Sci. USA, 96:14694 (1999).

Luthi-Peng et al., FEBS Lett., 299:111 (1992).

MacDonald et al., J. Physiol., 417:483 (1989).

Marks et al., J. Clin. Invest., 96:2630 (1995).

Másson et al., Die Pharmazie, 55:172 (2000).

Menendez et al., Int. J. Oncol., 24:591 (2004).

Miller et al., Chemistry & Biology, 9:333 (2002).

Nakagawa et al., Genome Res., 13:1042 (2003).

Nakamura et al., Int. J. Mol. Med., 4, 381 (1999).

Olsen et al., J. Neurosci., 2:1812 (1982).

Parish et al., J. Bacterial., 179:7827 (1997).

Physician's Desk Reference (PDR, Medical Economics Company (Montvale, N.J.), (53rd Ed.), pp. 1059-1067.

Physician's Desk Reference, 2001 Edition.

Pizer et al., Cancer Res. 56:1189 (1996).

Porterin et al., Proc. Natl. Acad. Sci. USA, 101:314 (2004).

Pro-drugs: Topical and Ocular Drug Delivery, Drugs and the Biological agent Sciences, Vol. 53, Kenneth B. Sloan (editor), Marcel Dekker (1992).

Protecting Groups in Organic Synthesis, Second Edition, Greene, T. W., and Wutz, P. G. M., John Wiley & Sons, New York.

Quemard et al., Biochemistry 34:8235 (1995).

Remington's Pharmaceutical Sciences, 17th ed., Mack Publishing Company, Easton, Pa, p. 1418 (1985).

Richards et al., Br. J. Pharmacol., 58:347 (1976).

Rossi et al., Mol. Cancer Res., 1:707 (2003).

Schubert et al., Infect. Imm., 70:5335 (2002).

Schubert et al., Adv. Exp. Med. & Biol., 485:69 (2000).

Schubert et al., Infect. Imm., 66:480 (1998).

Stevens et al., J. Infect. Dis., 179:S366 (1999).

Stinear et al., Proc. Natl. Acad. Sci. USA, 101:1345 (2004).

Swinnen et al., Int. J. Cancer, 98:19 (2002).

Textbook of Drug Design and Development, Hans Bundgaard (editor), Hardwood Academic Pub (1991).

Tsuji et al., Acta Obstet. Gynecol. Scand., 83:586 (2004).

Tucker et al., J. Med. Chem., 37:2437 (1994).

Vilcheze et al., J. Bacteriol., 182:4059 (2000).

Wakil, Biochemistry, 28:4523 (1989).

Wang et al., J. Exp. Ther. Oncol., 4:101 (2004).

Yamada et al., BBRC, 299:49 (2002).

Yamada et al., J. Biochem. (Tokyo), 126:1013 (1999).

Yuan et al., J. Biol. Chem., 261:13643 (1986).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed:

1. A compound of formula (I)-(XIII).

2. A compound of formula (I)-(XIII), for use in medical therapy or diagnosis.

3. The use of a compound of formula (I)-(XIII), for the manufacture of a medicament for treating cancer.

4. A pharmaceutical composition comprising a pharmaceutically acceptable carrier and a compound of formula (I)-(XIII).

5. A method of inhibiting fatty acid synthase (FAS), the method comprising contacting the FAS with an effective amount of a compound of formula (I)-(XIII).

6. The method of claim 5, wherein the contacting is in vivo.

7. The method of claim 5, wherein the contacting is in vitro.

8. The method of any one of claims 5-7, wherein the thioesterase (TE) domain of the FAS is inhibited.

9. A method of treating cancer in a mammal, the method comprising administering to a mammal in need of such treatment an effective amount of a compound of formula (I)-(XIII).

10. The method of claim 9, wherein the mammal is a human.

11. A method of inhibiting tumor cell growth in a mammal, the method comprising administering to a mammal in need of such treatment an effective amount of a compound of formula (I)-(XIII).

12. The method of claim 11, wherein the mammal is a human.

13. The method of any one of claims 11-12, wherein the tumor is a solid tumor.

14. The method of any one of claims 11-13, wherein the tumor is located in the ovary, breast, lung, thyroid, lymph node, kidney, ureter, bladder, ovary, teste, prostate, bone, skeletal muscle, bone marrow, stomach, esophagus, small bowel, colon, rectum, pancreas, liver, smooth muscle, brain, spinal cord, nerves, ear, eye, nasopharynx, oropharynx, salivary gland, or the heart.

15. The method of any one of claims 11-14, wherein the administration is systemic.

16. The method of any one of claims 9-15, further comprising administering one or more anti-cancer agents.

17. A method of inhibiting or treating an infection of a mammal by a pathogen, comprising: administering to the mammal an effective amount of an agent that is a selective inhibitor of one or more pathogen-specific polypeptides containing a TE domain.

18. The method of claim 17 wherein the pathogen is E. coli.

19. The method of claim 17 wherein the pathogen is Yersinia pestis.

20. The method of any one of claims 17-19 wherein the inhibitor inhibits YbtT about 2-fold greater than human FAS.

21. A method to identify an agent that is selective inhibitor of a TE domain in a polypeptide, comprising:
a) comparing percent inhibition of a prokaryotic polypeptide having a TE domain by an agent to the percent inhibition of a eukaryotic polypeptide having a TE domain by the agent; and
b) identifying whether the agent selectively inhibits the prokaryotic polypeptide having a TE domain or the eukaryotic polypeptide having a TE domain.

22. A method of inhibiting angiogenesis in a mammal, the method comprising administering an effective amount of an antagonist of fatty acid synthase to the mammal, thereby effectively inhibiting angiogenesis in the mammal.

23. The method of claim 22, wherein the mammal is a human.

24. The method of any one of claims 22-23, wherein the fatty acid synthase antagonist is a compound of formula (I)-(XIII).

25. The method of any one of claims 22-24, wherein the inhibiting angiogenesis effectively treats one or more of cancer, macular degeneration, diabetic retinopathy, arthritis, obesity, psoriasis, eczema, scleroderma, a haemangioma, an angiosarcoma, and Kaposi's sarcoma in the mammal.

26. A method of inhibiting fat deposition, obesity, or a combination thereof in a mammal, the method comprising inhibiting fatty acid synthesis in a mammal.

27. The method of claim 26, wherein the fatty acid synthase is inhibited by administering an effective amount of a compound of formula (I)-(XIII) to the mammal.

28. The method of any one of claims 26-27, wherein the mammal is a human.

29. The method of any one of claims 26-28, wherein the thioesterase (TE) domain of the FAS is inhibited.
